SEQUENCE LISTING (36 sequences)

SEQ ID NO: 1 - DNA, 19 bp, Artificial Sequence
  Synthetic polynucleotide
agacatcgccagtggcatg

SEQ ID NO: 2 - DNA, 20 bp, Artificial Sequence
  Synthetic polynucleotide
aggcgatttcctgcttcagg

SEQ ID NO: 3 - DNA, 23 bp, Artificial Sequence
  Synthetic polynucleotide
catcaacaagtatgggtctccgt

SEQ ID NO: 4 - DNA, 23 bp, Artificial Sequence
  Synthetic polynucleotide
gttctcctggctgcattcgttga

SEQ ID NO: 5 - DNA, 20 bp, Artificial Sequence
  Synthetic polynucleotide
tgaagaaagtcccttcgtgg

SEQ ID NO: 6 - DNA, 20 bp, Artificial Sequence
  Synthetic polynucleotide
gatctgtccatcccgaagcc

SEQ ID NO: 7 - DNA, 20 bp, Artificial Sequence
  Synthetic polynucleotide
tgtcagacgatgggatgggc

SEQ ID NO: 8 - DNA, 20 bp, Artificial Sequence
  Synthetic polynucleotide
gcgtctccacaggaagccag

SEQ ID NO: 9 - DNA, 20 bp, Artificial Sequence
  Synthetic polynucleotide
tggtagtcaggtaccgcgtg

SEQ ID NO: 10 - DNA, 21 bp, Artificial Sequence
  Synthetic polynucleotide
tccagctctgacctcgtgcag

SEQ ID NO: 11 - DNA, 22 bp, Artificial Sequence
  Synthetic polynucleotide
atatccgggcgtggagaacagc

SEQ ID NO: 12 - DNA, 20 bp, Artificial Sequence
  Synthetic polynucleotide
gaatccttagggtctggctg

SEQ ID NO: 13 - DNA, 28 bp, Artificial Sequence
  Synthetic polynucleotide
ctgcgtgagcttgttactcgtgccttgg

SEQ ID NO: 14 - DNA, 20 bp, Artificial Sequence
  Synthetic polynucleotide
agcagtcactgggggacttg

SEQ ID NO: 15 - DNA, 21 bp, Artificial Sequence
  Synthetic polynucleotide
ggtgcaggagaggagaactgc

SEQ ID NO: 16 - DNA, 20 bp, Artificial Sequence
  Synthetic polynucleotide
ggttttctgaccggaggtcc

SEQ ID NO: 17 - DNA, 21 bp, Artificial Sequence
  Synthetic polynucleotide
aggaccaagcaacatggtcag

SEQ ID NO: 18 - DNA, 21 bp, Artificial Sequence
  Synthetic polynucleotide
tgcatccgtaggtgcagtttg

SEQ ID NO: 19 - DNA, 21 bp, Artificial Sequence
  Synthetic polynucleotide
gatggtgggggccctcctctt

SEQ ID NO: 20 - DNA, 21 bp, Artificial Sequence
  Synthetic polynucleotide
tccgggaacacaaagacaata

SEQ ID NO: 21 - DNA, 24 bp, Artificial Sequence
  Synthetic polynucleotide
ctttctcttccgcacccagcagtt

SEQ ID NO: 22 - DNA, 24 bp, Artificial Sequence
  Synthetic polynucleotide
atccatcagggcacggtagaagtt

SEQ ID NO: 23 - DNA, 21 bp, Artificial Sequence
  Synthetic polynucleotide
agtgctggatgatagacgcag

SEQ ID NO: 24 - DNA, 21 bp, Artificial Sequence
  Synthetic polynucleotide
gtcaacagcacattcgacagc

SEQ ID NO: 25 - DNA, 21 bp, Artificial Sequence
  Synthetic polynucleotide
aaattcactgctttgtggcgc

SEQ ID NO: 26 - DNA, 3275 bp, Homo sapiens
ggcggctgctgggcagagccggtggcaagggcctcccctgccgctgtgccaggcaggcagtgccaaatccggggagcctggagctggggggagggccggggacagcccggccctgccccctcccccgctgggagcccagcaacttctgaggaaagtttggcacccatggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagggcaagcacagccagtccaccagctggtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcttgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccggggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctgagacaaccctccacctggtactccctctcaggatccaagctaagcactgccactggggaaaactccaccttcccactttcccaccccacgccttatccccacttgcagccctgtcttcctacctatcccacctccatcccagacaggtccctccccttctctgtgcagtagcatcaccttgaaagcagtagcatcaccatctgtaaaaggaaggggttggattgcaatatctgaagccctcccaggtgttaacattccaagactctagagtccaaggtttaaagagtctagattcaaaggttctaggtttcaaagatgctgtgagtctttggttctaaggacctgaaattccaaagtctctaattctattaaagtgctaaggttctaaaaaaaaaaaaaaaaaaaaaaaaaaaaaaaaaaaaaaaa

SEQ ID NO: 27 - DNA, 2685 bp, Homo sapiens
atggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagggcaagcacagccagtccaccagctggtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcttgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccggggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctga

SEQ ID NO: 28 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRRGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 29 - DNA, 2461 bp, Homo sapiens
ccgcagccgccgccgccgccgccgccgcgatgtgaccttcagggccgccaggacgggatgaccggagcctccgccccgcggcgcccgctcgcctcggcctcccgggcgctctgaccgcgcgtccccggcccgccatggccccttcgctctcgcccgggcccgccgccctgcgccgcgcgccgcagctgctgctgctgctgctggccgcggagtgcgcgcttgccgcgctgttgccggcgcgcgaggccacgcagttcctgcggcccaggcagcgccgcgcctttcaggtcttcgaggaggccaagcagggccacctggagagggagtgcgtggaggagctgtgcagccgcgaggaggcgcgggaggtgttcgagaacgaccccgagacggattatttttacccaagatacttagactgcatcaacaagtatgggtctccgtacaccaaaaactcaggcttcgccacctgcgtgcaaaacctgcctgaccagtgcacgcccaacccctgcgataggaaggggacccaagcctgccaggacctcatgggcaacttcttctgcctgtgtaaagctggctgggggggccggctctgcgacaaagatgtcaacgaatgcagccaggagaacgggggctgcctccagatctgccacaacaagccgggtagcttccactgttcctgccacagcggcttcgagctctcctctgatggcaggacctgccaagacatagacgagtgcgcagactcggaggcctgcggggaggcgcgctgcaagaacctgcccggctcctactcctgcctctgtgacgagggctttgcgtacagctcccaggagaaggcttgccgagatgtggacgagtgtctgcagggccgctgtgagcaggtctgcgtgaactccccagggagctacacctgccactgtgacgggcgtgggggcctcaagctgtcccaggacatggacacctgtgaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcctaggcccccacgggacgcggcaggcttctcagtctctgtccgagacagccgggaggagcctgggggctcctcaccacgtggggccatgctgagagctgggctttcctctgtgaccatcccggcctgtaacatatctgtaaatagtgagatggacttggggcctctgacgccgcgcactcagccgtgggcccgggcgcggggaggccggcgcagcgcagagcgggctcgaagaaaataattctctattatttttattaccaagcgcttctttctgactctaaaatatggaaaat

SEQ ID NO: 30 - DNA, 2037 bp, Homo sapiens
atggccccttcgctctcgcccgggcccgccgccctgcgccgcgcgccgcagctgctgctgctgctgctggccgcggagtgcgcgcttgccgcgctgttgccggcgcgcgaggccacgcagttcctgcggcccaggcagcgccgcgcctttcaggtcttcgaggaggccaagcagggccacctggagagggagtgcgtggaggagctgtgcagccgcgaggaggcgcgggaggtgttcgagaacgaccccgagacggattatttttacccaagatacttagactgcatcaacaagtatgggtctccgtacaccaaaaactcaggcttcgccacctgcgtgcaaaacctgcctgaccagtgcacgcccaacccctgcgataggaaggggacccaagcctgccaggacctcatgggcaacttcttctgcctgtgtaaagctggctgggggggccggctctgcgacaaagatgtcaacgaatgcagccaggagaacgggggctgcctccagatctgccacaacaagccgggtagcttccactgttcctgccacagcggcttcgagctctcctctgatggcaggacctgccaagacatagacgagtgcgcagactcggaggcctgcggggaggcgcgctgcaagaacctgcccggctcctactcctgcctctgtgacgagggctttgcgtacagctcccaggagaaggcttgccgagatgtggacgagtgtctgcagggccgctgtgagcaggtctgcgtgaactccccagggagctacacctgccactgtgacgggcgtgggggcctcaagctgtcccaggacatggacacctgtgaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcctag

SEQ ID NO: 31 - protein, 678 aa, Homo sapiens
MAPSLSPGPAALRRAPQLLLLLLAAECALAALLPAREATQFLRPRQRRAFQVFEEAKQGHLERECVEELCSREEAREVFENDPETDYFYPRYLDCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 32 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 33 - DNA, 3633 bp, Homo sapiens
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 34 - protein, 1210 aa, Homo sapiens [sequence omitted]

SEQ ID NO: 35 - protein, 302 aa, Homo sapiens
GSVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHEF

SEQ ID NO: 36 - protein, 327 aa, Homo sapiens
GEAPNQALLRILKETEFKKIKVLGSGAFGTVYKGLWIPEGEKVKIPVAIKELREATSPKANKEILDEAYVMASVDNPHVCRLLGICLTSTVQLIMQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQG

(19) United States
(12) Patent Application Publication
(10) Pub. No.: US 2014/0121126 A1
(43) Pub. Date: May 1, 2014

(54) METHODS OF DETECTING AXL AND GAS6 IN CANCER PATIENTS

(71) Applicants: Memorial Sloan-Kettering Cancer Center, New York, NY (US); The Regents of the University of California, Oakland, CA (US)

(72) Inventors: Trever G. Bivona, San Francisco, CA (US); Charles Sawyers, New York, NY (US)

(73) Assignees: Memorial Sloan-Kettering Cancer Center, New York, NY (US); The Regents of the University of California, Oakland, CA (US)

(21) Appl. No.: 14/064,042

(22) Filed: Oct. 25, 2013

Related U.S. Application Data
(60) Provisional application No. 61/718,560, filed on Oct. 25, 2012.

(51) Int. Cl. G01N 33/68; C12Q 1/68

(52) U.S. Cl. CPC G01N 33/68; C12Q 1/6886
USPC 506/9; 435/6.12; 436/501; 435/7.92; 435/15

(57) ABSTRACT

Described herein, inter alia, are compositions and methods for detecting levels of AXL and GAS6.

### CROSS-REFERENCES TO RELATED APPLICATIONS

This application claims the benefit of U.S. Provisional Patent Application No. 61/718,560, filed Oct. 25, 2012, which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 84850-891535_ST25.TXT, created on Oct. 23, 2013, 60,966 bytes, machine format IBM-PC, MS-Windows operating system, is hereby incorporated by reference.

### BACKGROUND OF THE INVENTION

Non-small cell lung cancer (NSCLC) has served as a model for genotype-directed targeted cancer therapy. NSCLC patients whose tumors harbor activating kinase domain mutations in the epidermal growth factor receptor (EGFR) often initially respond to treatment with an EGFR tyrosine kinase inhibitor (TKI) such as erlotinib. [Paez, J. G. et al., Science 304, 1497-1500 (2004); Pao, W. et al., Proc Natl Acad Sci USA 101, 13306-13311 (2004); Lynch, T. J. et al., The New England journal of medicine 350, 2129-2139 (2004); Sordella, R., Bell, D. W., Haber, D. A. & Settleman, J., Science 305, 1163-1167, (2004)] However, acquired resistance to EGFR TKI treatment invariably develops. [Janne, P. A., Gray, N. & Settleman, J., Nat Rev Drug Discov 8, 709-723 (2009); Gazdar, A. F., The New England journal of medicine 361, 1018-1020 (2009)] There is no effective therapy for patients who develop such resistance. It has been shown that resistance to EGFR TKI treatment can occur through a secondary resistance mutation in EGFR (T790M), activation of the MET kinase, and activation of the NF-kB pathway. [Kobayashi, S. et al. N Engl J Med 352, 786-792 (2005); Pao, W. et al. PLoS Med 2, e73 (2005); Engelman, J. A. et al. Science 316, 1039-1043 (2007); Turke, A. B. et al. Cancer Cell 17, 77-88 (2010); Bivona, T. G. et al. Nature 471, 523-526 (2011); Arcila, M. E. et al. Clinical cancer research: an official journal of the American Association for Cancer Research 17, 1169-1180 (2011)] The mechanisms underlying acquired resistance to EGFR TKI treatment are unknown in over 40% of EGFR-mutant NSCLC patients.

Recent studies indicate that multiple resistance mechanisms may operate within an individual tumor to promote EGFR TKI acquired resistance in NSCLC patients. For example, the EGFR T790M resistance mutation and activation of MET can co-occur in some EGFR-mutant NSCLCs with acquired resistance to EGFR TKI treatment. [Bean, J. et al. Proc Natl Acad Sci USA 104, 20932-20937 (2007); Arcila, M. E. et al. Clinical cancer research: an official journal of the American Association for Cancer Research 17, 1169-1180 (2011)] Furthermore, recent evidence suggests that the acquisition of EGFR TKI resistance in EGFR-mutant NSCLCs may be associated not only with genotypic alterations but also histological changes that occur during adaptation to therapy. [Sequist, L. V. et al. Sci Transl Med 3, 75ra26 (2011)] There is a need in the art for identifying novel mechanisms of acquired resistance to EGFR TKI treatment, clarifying the extent to which distinct and co-existent genotypic and histological changes promote the acquisition of EGFR TKI treatment resistance in NSCLC patients, and for effective compositions and methods for overcoming resistance to EGFR TKI treatment. The present invention provides solutions to these and other problems in the art.

### BRIEF SUMMARY OF THE INVENTION

In an aspect is provided a method of treating epidermal growth factor receptor (EGFR) inhibitor resistant cancer. The method includes detecting an increased level of AXL or GAS6 in a patient sample relative to a control (e.g. a control sample) and administering a therapeutically effective amount of an AXL inhibitor to the patient.

In another aspect is provided a method of detecting AXL or GAS6 levels in a cancer patient. The method includes contacting a sample from the cancer patient with a detectable AXL-binding agent or detectable GAS6-binding agent, allowing the detectable AXL-binding agent or the detectable GAS6-binding agent to bind to AXL or GAS6, respectively, allowing the detectable AXL-binding agent and AXL to form an AXL complex or the GAS6-binding agent and GAS6 to form a GAS6 complex, and detecting the AXL complex or GAS6 complex.

In another aspect is provided a method of identifying an EGFR inhibitor resistant cancer patient. The method includes obtaining a sample from a plurality of cancer patients, detecting a level of AXL or a level of GAS6 in each of the samples, comparing the level of AXL or the level of GAS6 to a control, identifying at least one sample from the plurality of cancer patients having a level of AXL or a level of GAS6 greater than the control thereby identifying an EGFR inhibitor resistant cancer patient.

In another aspect is provided a method of treating EGFR inhibitor resistant cancer. The method includes detecting an increased level of AXL activity in a patient (e.g. from a patient sample) relative to a control (e.g. control sample) and administering a therapeutically effective amount of an AXL inhibitor to the patient.

In another aspect is provided a method of identifying an EGFR inhibitor resistant cancer patient including obtaining a sample from a plurality of cancer patients, detecting a level of AXL activity in each of the samples, comparing the level of AXL activity to a control, identifying at least one sample from the plurality of cancer patients having a level of AXL activity greater than the control thereby identifying an EGFR inhibitor resistant cancer patient.

In another aspect is provided a pharmaceutical composition including a combined therapeutically effective amount of an AXL inhibitor and an EGFR inhibitor.

In another aspect is provided a method of detecting a level of AXL or GAS6 in a subject (e.g. a cancer patient), the method including: (i) obtaining a sample from a subject (e.g. cancer patient); and (ii) detecting a differential expression level of AXL or GAS6 in the sample relative to a control.

In another aspect is provided a method of detecting an increased level of AXL or GAS6 in a subject (e.g. cancer patient), the method including: (i) obtaining a sample from a subject (e.g. cancer patient); and (ii) detecting an increased level of AXL or GAS6 in the sample relative to a control.

In another aspect is provided a method of identifying an EGFR inhibitor resistant cancer patient including: (i) obtaining a sample from a cancer patient; and (ii) detecting an increased level of AXL or GAS6 in the sample relative to a control.

### BRIEF DESCRIPTION OF THE DRAWINGS

FIG. 1. AXL is overexpressed in EGFR-mutant NSCLC tumor xenografts with acquired resistance to erlotinib. a, Effects of a dose-response of erlotinib in HCC827 xenograft tumors in immunocompromised mice (n=10 tumors/treatment group). b-f, mRNA expression of indicated genes in HCC827 erlotinib resistant tumor xenografts (treated at the indicated erlotinib doses) relative to vehicle-treated control tumors. The number of tumors analyzed from each treatment cohort is indicated in parentheses. Data are expressed as the mean±SEM of the fold change relative to the mean expression of the genes in 2 vehicle treated control xenograft tumors.

FIG. 2. AXL overexpression is necessary for acquired resistance to erlotinib treatment in EGFR-mutant NSCLC tumors in vivo. a, AXL and pAXL protein levels are increased in the absence of pEGFR and increased pMET in HCC827 tumors from each erlotinib treatment cohort compared with vehicle treated tumors. Tumors were harvested for analysis at the completion of the study for each treatment group (Vehicle: Day 45; 6.25 Erl: Day 60; 12.5 Erl: Day 110; 25 Erl: Day 110; 50 Erl: Day 100) b, Acute and transient treatment of HCC827 tumors with erlotinib (12.5 mg/kg/day) decreases pAKT, pERK, pRELa and increases the levels of cleaved Parp. c, Response of parental HCC827 xenograft tumors or HCC827 ER xenograft tumors (n=10 tumors/group) transduced with a non-target shRNA or an shRNA targeting AXL to treatment with either vehicle or erlotinib (12.5 mg/kg/day). Tumor volumes are expressed as mean±SEM. d, Validation of AXL knockdown and the effects of erlotinib treatment on pEGFR in representative tumor xenografts (c) by western blot analysis.

FIG. 3. AXL upregulation is necessary and sufficient for erlotinib acquired resistance in EGFR mutant NSCLC cellular models. a, HCC827 ER1-ER5 sublines are resistant to erlotinib treatment as measured by CellTiterGLO cell viability assay. Data are from 3 independent experiments and are expressed as percent of vehicle treated cells and mean±SEM. b, Expression of AXL and GAS6 in the ER sublines compared with parental HCC827 cells (data are from Western blot analysis). c, Erlotinib IC₅₀ in HCC827 cell lines (as indicated) measured 48 h after treatment with a non-targeting or AXL or GAS6 siRNA. Erlotinib IC₅₀ is shown in parentheses. Data are representative of 3 independent experiments. d, Erlotinib IC₅₀ in HCC827 cell lines (as indicated) measured 48 h after treatment with vehicle (control) or with MP-470 (1 μM) or XL-880 (1 μM) and erlotinib. Erlotinib IC₅₀ is shown in parentheses. Data are representative of 3 independent experiments. e, Effects of treatment for 48 h with a non-targeting (−) or AXL siRNA in parental or ER1 and ER2 cell lines in the absence and presence of erlotinib on the indicated biomarkers. Data represent 3 independent experiments. f-g, Effects of treatment for 48 h with a vehicle or the indicated doses of (f) MP-470 or (g) XL-880 in parental or ER1 and ER2 cell lines in the absence and presence of erlotinib on the indicated biomarkers. Data represent 3 independent experiments. h, Erlotinib IC₅₀ in HCC827 cells measured 5 days after transfection the cDNA constructs encoding the indicated proteins and treated with either vehicle (left) or with XL-880 (1 μM) and erlotinib. Erlotinib IC₅₀ is shown in parentheses. Data are representative of 3 independent experiments. i, Western blot for the indicated proteins in lysates from cells transfected with the indicated cDNA constructs and treated with XL-880 (1 μM) for 3 hours prior to cell lysis (h).

FIG. 4. AXL-mediated erlotinib resistance occurs in association with EMT in EGFR-mutant NSCLC cellular models. a, Effects of treatment with a non-targeting or the indicated vimentin siRNAs on AXL expression in HCC827 ER3 cells. b, Erlotinb IC₅₀ in HCC827 parental or ER3 cells upon treatment with a non-targeting or the indicated vimentin siRNAs. Erlotinib IC₅₀ is shown in parentheses. Data represent 3 independent experiments. c, Increased migration through a transwell chamber of ER3 compared to parental HCC827 cells in cells treated with a non-targeting or the indicated vimentin (VIM) or AXL siRNAs or XL-880 (1 μM); n=3, data expressed as mean±SEM. d, Increased adherence to plastic of HCC827 ER3 cells compared to HCC827 parental cells in cells treated with a non-targeting or the indicated vimentin (VIM) or AXL siRNAs or XL-880 (1 μM). 5 100× microscopic fields per cell line were counted. n=3, data expressed as mean±SEM. e, Western blot for the indicated proteins in lysates from cells transfected with the indicated siRNAs in (c-d).

FIG. 5. AXL upregulation occurs in human EGFR-mutant NSCLCs with EGFR TKI acquired resistance. a-b, Representative expression of the indicated proteins by IHC in (a) case 7 and (b) case 9 shown in Table 2. IHC staining for AXL and GAS6 was scored as shown in FIG. 19. Vimentin IHC staining was scored using an established, clinically validated protocol [Azumi, N. & Battifora, H. American journal of clinical pathology 88, 286-296 (1987)] and EGFR T790M and MET amplification were assessed by sequencing and FISH, respectively, using established assays. Scale bars indicate in (a) 100 μM and (b) 10 μM.

FIG. 6. Increased AXL and GAS6 levels were not observed in HCC827 tumors or cell lines following acute (48 h) treatment with erlotinib. mRNA expression of AXL and GAS6 was measured by Q-RT-PCR and is expressed in erlotinib treated samples relative to vehicle treated samples. n=3 for xenograft and cell line analysis. Data are expressed as mean±SEM.

FIG. 7. mRNA expression of vimentin in HCC827 erlotinib resistant tumor xenografts (treated at the indicated erlotinib doses) relative to vehicle-treated control tumors. The number of tumors analyzed from each treatment cohort is indicated in parentheses. Data are expressed as the mean±SEM of the fold change relative to the mean expression of the genes in 2 vehicle treated control xenograft tumors.

FIG. 8. HCC827 ER1-ER5 sublines are resistant to treatment with the irreversible EGFR TKI BIBW2992, as measured by CellTiterGLO cell viability assay. Data are from 3 independent experiments and are expressed as percent of vehicle treated cells and mean±SEM.

FIG. 9. Expression of the indicated genes by Q RT PCR in the indicated HCC827 cell lines. Data are from 3 independent experiments and are actin normalized and expressed relative to parental cells as the mean±SEM.

FIG. 10. a-b, Effect of treatment with non-target, AXL, MET or AXL and MET siRNAs on erlotinib sensitivity in (a) HCC827 parental or (b) ER1 cells, as measured by CellTiterGLO viability assay. Data are from 3 independent experiments and are expressed as percent of vehicle treated cells and mean±SEM. c, Effect of treatment with non-target, AXL, MET or AXL and MET siRNAs and erlotinib on the indicated signaling biomarkers by western blot on lysates from treated HCC827 parental, ER1 and ER2 cells. Data represent 3 independent experiments.

FIG. 11. a-f, Effect of AXL knockdown on the indicated human NSCLC cell lines that express wild type EGFR. n=3, data expressed as mean±SEM.

FIG. 12. Effect of single-agent treatment with either vehicle or MP-470 or XL-880, each at 1 mM, on cell viability in the indicated cell lines as measured by CellTiterGLO assay. n=3, data expressed as mean±SEM.

FIG. 13. a-d, Combination treatment with XL880 and erlotinib, but not PHA and erlotinib, leads to a synergistic decrease in cell viability by combination index analysis. CI values of less than 1, 1, and greater than 1 indicate synergism, additive effect, and antagonism, respectively. The hashed line marks CI=1. Data are from 3 independent experiments.

FIG. 14. a-b, Effects of erlotinib treatment on the indicated biomarkers of pathway activation as measured by western blot on lysates from HCC827 or ER3 cells treated with (a) erlotinib and EGF or (b) erlotinib over a time course. c-d, Effects of AXL inhibition by (c) siRNA or (d) XL880 on the indicated biomarkers of pathway activation as measured by western blots on lysates from HCC827 or ER3 cells. e-f, Expression of AXL by (e) Q RT PCR or (f) western blot on lysates from ER4 and ER5 cells. g, Expression of the indicated proteins by western blot on lysates from ER4 and ER5 cells. h-k, Effects of AXL inhibition by (h,j) siRNA or (i,k) XL880 on the indicated biomarkers of pathway activation as measured by western blots on lysates from HCC827 on (h-i) ER4 or (j-k) ER5 cells treated with vehicle or erlotinib at the indicated doses.

FIG. 15. AXL overexpression is necessary for erlotinib resistance in H3255 EGFR-mutant NSCLC cells with acquired resistance to erlotinib. a, AXL is overexpressed in the absence of increased pEGFR or pMET. Increased expression of the EMT marker vimentin was also noted in conjunction with AXL overexpression. b, Knockdown of AXL by siRNA restored erlotinib sensitivity in the H3255 ER subclones (erlotinib IC₅₀ shown in mM). c, Validation of siRNA knockdown of AXL in H3255 ER cells as measured by Q-RT-PCR and normalized to H3255 ER cells treated with not-target control. Data are from 3 independent experiments and as mean±SEM. d, Erlotinib IC₅₀ in H3255 cells treated with MP-470 or XL-880 at 1 mM concentration. Data represent 3 independent experiments.

FIG. 16. Effects of expression of the indicated cDNA constructs encoding AXL or mutants thereof on XL-880 IC₅₀ in HCC827 parental or ER3 cells. Data represent 3 independent experiments.

FIG. 17. Effects of ectopic expression of AXL in PC9 cells on (a) erlotinib sensitivity as measured by CellTiterGLO viability assay. n=3, data expressed an mean±SEM. (b) Western blots for the indicated proteins performed on lysates generated from the parental or AXL overexpressing cells (c1) shown in (a).

FIG. 18. Structural modeling using PDB viewer of the gatekeeper residue in the AXL kinase domain that is predicted to interact with XL880 based on structural analogy to the EGFR T790M gatekeeper residue and to c-MET/XL-880 co-crystal structure. Sequence legend: PDB 3LQ8 (SEQ ID NO:35); PDB 2JIT (SEQ ID NO:36).

FIG. 19. Validation of IHC scoring system for the proteins examined in the paired human EGFR-mutant NSCLC specimens from EGFR TKI treated patients in Tables 2-3 and FIG. 5a-b. Scale bar=100 μM.

FIG. 20 Crizotinib overcomes EGFR TKI resistance in the ER models of acquired resistance. Crizotinib Used for treatment of NSCLC characterized by EML4-ALK fusion. Inhibits multiple kinases, including ALK, MET, and AXL. Crizotinib confers dose-dependent sensitivity to erlotinib in ER cells at 10 μM.

FIG. 21. Overcoming acquired resistance through AXL inhibitor treatment. Erlotinib plus AD57, AD80, or AD81 in ER4 cells (ER4 subline of HCC827 cells that are resistant to erlotinib treatment). Legend: diamond symbols are DMSO control, square symbols are 0.1 micromolar of compound, triangle symbols are 1 micromolar of compound, x symbol are 10 micromolar of compound, all in combination with erlotinib. AD57 and AD80 confer dose-dependent sensitivity to erlotinib in ER4 cells.

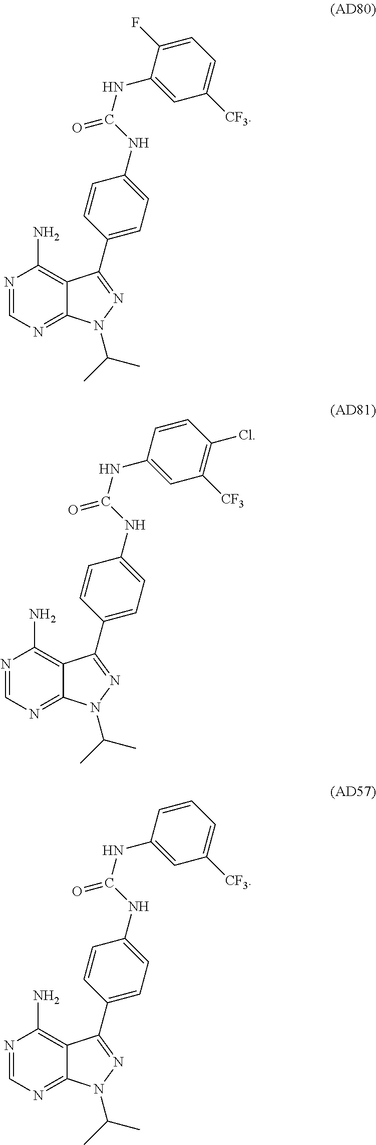 [SMILES: CC(C)n1nc(-c2ccc(NC(=O)Nc3cc(C(F)(F)F)ccc3F)cc2)c2c(N)ncnc21 ; CC(C)n1nc(-c2ccc(NC(=O)Nc3ccc(Cl)c(C(F)(F)F)c3)cc2)c2c(N)ncnc21 ; CC(C)n1nc(-c2ccc(NC(=O)Nc3cccc(C(F)(F)F)c3)cc2)c2c(N)ncnc21]

FIG. 22. Induction of apoptosis by combined EGFR and AXL inhibition. ER4 cells (ER4 subline of HCC827 cells that are resistant to erlotinib treatment) plated at 0.5×10⁶ cells/condition. 24-hour drug exposure. Apoptosis measured by induction of PARP cleavage and BIM induction. E is erlotinib, AD57 at 1 micromolar, AD80 at 1 micromolar, AD81 at 10 micromolar. 24 hour exposure to AD57 or AD80 combined with erlotinib enhances apoptosis in ER4 cells.

FIG. 23. List of genes with accompanying gene symbols, accession numbers, and/or Affymetrix probe numbers for identifying the genes making up an EMT signature, including AXL, by which epithelial cells and mesenchymal cells may be differentiated; 35 marker EMT signature.

FIG. 24. List of genes with accompanying gene symbols, accession numbers, and/or Affymetrix probe numbers for identifying the genes making up an EMT signature, including AXL, by which epithelial cells and mesenchymal cells may be differentiated; 76 marker EMT signature.

### DETAILED DESCRIPTION OF THE INVENTION

### Definitions

The abbreviations used herein have their conventional meaning within the chemical and biological arts. The chemical structures and formulae set forth herein are constructed according to the standard rules of chemical valency known in the chemical arts.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The terms “a” or “an,” as used in herein means one or more. In addition, the phrase “substituted with a[n],” as used herein, means the specified group may be substituted with one or more of any or all of the named substituents.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease.

An “effective amount” is an amount sufficient for an active pharmaceutical agent (API) such as a modulator (e.g. inhibitor, compound) to accomplish a stated purpose relative to the absence of the API (e.g. modulator, inhibitor, compound) (e.g. achieve the effect for which it is administered, treat a disease, reduce enzyme activity, increase enzyme activity, reduce signaling pathway, reduce one or more symptoms of a disease or condition (e.g. reduce AXL activity in a cell, increase AXL activity, reduce signaling pathway stimulated by AXL or GAS6, reduce the signaling pathway activity of AXL, reduce the signaling pathway activity of GAS6, reduce EGFR inhibitor resistance). An example of an “effective amount” is an amount sufficient to contribute to the treatment, prevention, or reduction of a symptom or symptoms of a disease, which could also be referred to as a “therapeutically effective amount.” A “combined therapeutically effective amount” is a total amount of a plurality of API's that in aggregation is an “effective amount” as defined herein. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist (e.g. disrupt the protein-protein interaction between AXL and GAS6, disrupt the interaction between AXL and substrates phosphorylated by AXL). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g. activity, protein-protein interaction, signaling pathway) of a protein (e.g. AXL, GAS6, or EGFR) in the absence of a modulator (e.g. compound, drug, inhibitor) as described herein (including embodiments, examples). The control may be a sample derived from a patient or individual (“control sample”).

In some embodiments, the control or control sample is a sample from a patient without the disease being detected or treated. In some embodiments, the control or control sample is from a non-diseased tissue or non-disease cells of the same original (e.g. organ, cell type) as the diseased tissue or cells being compared to the control. In some embodiments, the control or control sample is from a different patient than the test sample. In some embodiments, the control or control sample is from the same patient as the test sample. In some embodiments, the control or control sample includes cancer cells that are not EGFR inhibitor resistant. In some embodiments, the control or control sample is a value or set of values determined from one or a plurality of samples without the disease being detected or treated. In some embodiments, the control or control sample is of cells or tissue prior to treatment with an EGFR inhibitor. In some embodiments, the control or control sample includes cancer cells sensitive to an EGFR inhibitor. In some embodiments, the control or control sample includes cells sensitive to erlotinib. In some embodiments, the control or control sample includes cells sensitive to gefitinib.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a modulator (e.g. compound, drug, inhibitor) as described herein and a protein or enzyme (e.g. AXL, GAS6, EGFR, or MET). In some embodiments, the protein may be AXL. In some embodiments, the protein may be a GAS6. In some embodiments, the protein may be EGFR. In some embodiments, the protein may be mutant EGFR. In some embodiments contacting includes allowing a modulator (e.g. compound, drug, or inhibitor) described herein to interact with a protein or enzyme that is involved in a signaling pathway. In some embodiments, contacting includes allowing a nucleic acid probe to interact with a target nucleic acid (e.g. AXL or GAS6 probe and AXL or GAS6 nucleic acid or fragment thereof).

As defined herein, the term “inhibition”, “inhibit”, “inhibiting” and the like in reference to a protein-inhibitor interaction means negatively affecting (e.g. decreasing) the activity or function of the protein (e.g. decreasing the signaling pathway stimulated by AXL, GAS6, EGFR or mutant EGFR) relative to the activity or function of the protein in the absence of the inhibitor (e.g. AXL, GAS6, EGFR or mutant EGFR). In some embodiments inhibition refers to reduction of a disease or symptoms of disease. In some embodiments, inhibition refers to a reduction in the activity of a signal transduction pathway or signaling pathway (e.g. reduction of a pathway involving AXL, reduction of a pathway involving mutant GAS6, reduction of a pathway involving EGFR). Thus, inhibition includes, at least in part, partially or totally blocking stimulation, decreasing, preventing, or delaying activation, or inactivating, desensitizing, or down-regulating the signaling pathway or enzymatic activity or the amount of a protein (e.g. AXL, GAS6, EGFR, mutant EGFR). In some embodiments, inhibition refers to inhibition of interactions of AXL with signaling pathway binding partners (e.g. GAS6, phosphorylation substrates). In some embodiments, inhibition refers to inhibition of interactions of AXL with a GAS6.

The term “modulator” refers to a composition that increases or decreases the level of a target molecule or the function (e.g. kinase activity, nucleotide exchange, effector protein binding, effector protein activation, phosphate release, nucleotide release, nucleotide binding) of a target molecule or the physical state (e.g. subcellular localization, post-translational processing, post-translational modifications) of the target of the molecule (e.g. a target may be AXL and the function may be to phosphorylate a substrate or activate a signaling pathway that is activated by AXL or GAS6, interaction of AXL with protein binding partners). In some embodiments, an AXL modulator is a compound that reduces the activity of AXL in a cell. In some embodiments, an AXL modulator is a compound that increases the activity of AXL in a cell. In some embodiments, an AXL modulator is a compound that reduces the signaling pathway in a cell that is activated by the AXL. In some embodiments, an AXL modulator is a compound that increases the signaling pathway in a cell that is activated by AXL. In some embodiments, an AXL disease modulator is a compound that reduces the severity of one or more symptoms of a disease associated with AXL (e.g. cancer, metastatic cancer). In some embodiments, an AXL modulator is a compound that increases or decreases the activity or function or level of activity or level of function of AXL or level of AXL or level of AXL in a particular physical state (e.g. phosphorylated AXL). In some embodiments, a mutant AXL modulator is a compound that increases or decreases the activity or function or level of activity or level of function of mutant AXL or level of mutant AXL or level of mutant AXL in a particular physical state. In some embodiments, an AXL modulator is a compound that reduces the activity of GAS6. In some embodiments, an AXL modulator is a compound that increases the activity of GAS6. In some embodiments, an AXL modulator is a compound that reduces the signaling pathway in a cell that is activated by GAS6. In some embodiments, an AXL modulator is a compound that increases the signaling pathway in a cell that is activated by GAS6. In some embodiments, an AXL disease modulator is a compound that reduces the severity of one or more symptoms of a disease associated with GAS6 (e.g. cancer, metastatic cancer). In some embodiments, an AXL modulator is a compound that increases or decreases the activity or function or level of activity or level of function of GAS6 or level of GAS6 or level of GAS6 in a particular physical state. In some embodiments, a mutant AXL modulator is a compound that increases or decreases the activity or function or level of activity or level of function of mutant GAS6 or level of mutant GAS6 or level of mutant GAS6 in a particular physical state. In some embodiments, an AXL modulator is an AXL inhibitor.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient” or “subject in need thereof” refers to a living organism suffering from or prone to a disease or condition that can be treated by administration of a modulator, drug, compound, or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with the modulators, inhibitors, drugs, compounds or methods provided herein. In some embodiments, the disease is a disease related to (e.g. caused by) AXL. In some embodiments, the disease is a disease related to (e.g. caused by) GAS6. In some embodiments, the disease is a disease related to (e.g. caused by) a mutant AXL, aberrant AXL signaling pathway activity, a mutant GAS6, or aberrant GAS6 signaling pathway activity (e.g. lung cancer, pancreatic cancer, breast cancer, colon cancer, esophageal cancer, thyroid cancer, liver cancer, glioblastoma, astrocytoma-glioblastoma, non-small cell lung cancer, EGFR inhibitor resistant forms of any of the cancers described herein). Examples of diseases, disorders, or conditions include, but are not limited to cancer. Examples of diseases, disorders, or conditions include, but are not limited to lung cancer, pancreatic cancer, breast cancer, colon cancer, esophageal cancer, thyroid cancer, liver cancer, glioblastoma, astrocytoma-glioblastoma, non-small cell lung cancer, EGFR inhibitor resistant cancer, EGFR inhibitor resistant lung cancer, EGFR inhibitor resistant pancreatic cancer, EGFR inhibitor resistant breast cancer, EGFR inhibitor resistant colon cancer, EGFR inhibitor resistant esophageal cancer, EGFR inhibitor resistant thyroid cancer, EGFR inhibitor resistant liver cancer, EGFR inhibitor resistant glioblastoma, EGFR inhibitor resistant astrocytoma-glioblastoma, and EGFR inhibitor resistant non-small cell lung cancer. In some instances, “disease” or “condition” refers to cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, lung, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), non-small cell lung cancer, or multiple myeloma.

As used herein, the term “cancer” refers to all types of cancer, neoplasm or malignant tumors found in mammals (e.g. humans), including leukemia, carcinomas and sarcomas. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

“AXL associated cancer” (also referred to herein as “AXL related cancer”) refers to a cancer caused by aberrant AXL activity or signaling (e.g. increased amount of AXL or GAS6, increased activity of AXL). A “cancer associated with aberrant AXL activity” (also referred to herein as “AXL related cancer”) is a cancer caused by aberrant AXL activity or signaling (e.g. a mutant AXL, increased amount of AXL or GAS6, increased activity of AXL, decreased activity of AXL, reduced amount of AXL or GAS6). AXL related cancers may include lung cancer, non-small cell lung cancer, pancreatic cancer, breast cancer, colon cancer, esophageal cancer, thyroid cancer, liver cancer, glioblastoma, astrocytoma-glioblastoma, EGFR inhibitor resistant cancer, EGFR inhibitor resistant lung cancer, EGFR inhibitor resistant pancreatic cancer, EGFR inhibitor resistant breast cancer, EGFR inhibitor resistant colon cancer, EGFR inhibitor resistant esophageal cancer, EGFR inhibitor resistant thyroid cancer, EGFR inhibitor resistant liver cancer, EGFR inhibitor resistant glioblastoma, EGFR inhibitor resistant astrocytoma-glioblastoma, and EGFR inhibitor resistant non-small cell lung cancer. In embodiments, any of the aforementioned AXL associated cancers may be associated with an EGFR having an activating mutation. Determining other cancers that are associated with aberrant activity of AXL or GAS6 is within the skill of a person of skill in the art.

As used herein, the term “disease-related cells” means cells that are associated with a disease or condition, which include but are not limited to cells that initiate a disease, cells that propogate a disease, cells that cause a disease, cells that cause one or more symptoms of a disease, cells that are a hallmark of a disease; cells that contain a particular protein or mRNA molecule that causes a symptom of the disease. In some embodiments, the disease is a cancer and the disease-related cell is a cancer cell. In some embodiments, the disease is a metastatic cancer and the disease-related cell is a metastatic cancer cell. In some embodiments, the disease is liver cancer and the disease-related cell is a liver cancer cell. In some embodiments, the disease is lung cancer and the disease-related cell is a lung cancer cell. In some embodiments, the disease is non-small cell lung cancer and the disease-related cell is a non-small cell lung cancer cell. In some embodiments, the disease is pancreatic cancer and the disease-related cell is a pancreatic cancer cell. In some embodiments, the disease is breast cancer and the disease-related cell is a breast cancer cell. In some embodiments, the disease is colon cancer and the disease-related cell is a colon cancer cell. In some embodiments, the disease is esophageal cancer and the disease-related cell is an esophageal cancer cell. In some embodiments, the disease is thyroid cancer and the disease-related cell is a thyroid cancer cell. In some embodiments, the disease is glioblastoma and the disease-related cell is a glioblastoma cell. In some embodiments, the disease is astrocytoma-glioblastoma and the disease-related cell is a astrocytoma-glioblastoma cell. In some embodiments, the disease is an EGFR inhibitor resistant cancer and the disease-related cell is an EGFR inhibitor resistant cancer cell. In some embodiments, the disease is an EGFR inhibitor resistant metastatic cancer and the disease-related cell is an EGFR inhibitor resistant metastatic cancer cell. In some embodiments, the disease is EGFR inhibitor resistant liver cancer and the disease-related cell is an EGFR inhibitor resistant liver cancer cell. In some embodiments, the disease is EGFR inhibitor resistant lung cancer and the disease-related cell is an EGFR inhibitor resistant lung cancer cell. In some embodiments, the disease is EGFR inhibitor resistant non-small cell lung cancer and the disease-related cell is an EGFR inhibitor resistant non-small cell lung cancer cell. In some embodiments, the disease is EGFR inhibitor resistant pancreatic cancer and the disease-related cell is an EGFR inhibitor resistant pancreatic cancer cell. In some embodiments, the disease is EGFR inhibitor resistant breast cancer and the disease-related cell is an EGFR inhibitor resistant breast cancer cell. In some embodiments, the disease is EGFR inhibitor resistant colon cancer and the disease-related cell is an EGFR inhibitor resistant colon cancer cell. In some embodiments, the disease is EGFR inhibitor resistant EGFR inhibitor resistant esophageal cancer and the disease-related cell is an EGFR inhibitor resistant esophageal cancer cell. In some embodiments, the disease is EGFR inhibitor resistant thyroid cancer and the disease-related cell is an EGFR inhibitor resistant thyroid cancer cell. In some embodiments, the disease is EGFR inhibitor resistant glioblastoma and the disease-related cell is an EGFR inhibitor resistant glioblastoma cell. In some embodiments, the disease is EGFR inhibitor resistant astrocytoma-glioblastoma and the disease-related cell is an EGFR inhibitor resistant astrocytoma-glioblastoma cell. In some embodiments, the disease is EGFR inhibitor resistant cancer and the EGFR has an activating mutation and the disease-related cell is an EGFR inhibitor resistant cell having an activating mutation.

The term “expression” refers to a gene that is transcribed or translated at a detectable level. As used herein, expression also encompasses “overexpression,” which refers to a gene that is transcribed or translated at a detectably greater level, usually in a cancer cell, in comparison to a normal cell. Expression therefore refers to both expression of AXL (or GAS6) protein and RNA, as well as local overexpression due to altered protein trafficking patterns and/or augmented functional activity. Expression can be detected using conventional techniques for detecting protein (e.g., ELISA, Western blotting, flow cytometry, immunofluorescence, immunohistochemistry, etc.) or mRNA (e.g., qPCR, RT-PCR, PCR, hybridization, etc.). One skilled in the art will know of other techniques suitable for detecting expression of AXL (or GAS6) protein or mRNA. Cancerous cells, e.g., lung cancer, non-small cell lung cancer, pancreatic cancer, breast cancer, colon cancer, esophageal cancer, thyroid cancer, liver cancer, glioblastoma, astrocytoma-glioblastoma, or EGFR inhibitor resistant forms thereof (e.g. having an EGFR-activating mutation), can express AXL (or GAS6) at a level of at least about 5%, 10%, 15%, 20%, 25%, 30%, 35%, 40%, 45%, 50%, 55%, 60%, 65%, 70%, 75%, 80%, 85%, 90%, 95%, 105%, 110%, 115%, 120%, 125%, 130%, 135%, 140%, 145%, 150%, 155%, 160%, 165%, 170%, 175%, 180%, 185%, 190%, 195%, in comparison to normal, non-cancerous cells. Cancerous cells can also have at least about a 1-fold, 2-fold, 3-fold, 4-fold, 5-fold, 6-fold, 7-fold, or higher level of AXL (or GAS6) transcription or translation in comparison to normal, non-cancerous cells. In certain instances, the cancer cell sample is autologous.

“Therapy resistant” cancers, tumor cells, and tumors refer to cancers that have become resistant (e.g. are not as susceptible to being treated by the resistant therapy compared to a non-resistant cancer, tumor cell, or tumor) to one or more cancer therapies (e.g. apoptosis-mediated, non-apoptosis-mediated, EGFR inhibitor treatment) including, but not limited to, chemotherapy, hormonal therapy, radiotherapy, immunotherapy, and combinations thereof.

“EGFR inhibitor” refers to an inhibitor (e.g. compound, antibody, drug) that treats a disease by targeting EGFR. In some embodiments, an EGFR inhibitor binds EGFR. In some embodiments, an EGFR inhibitor inhibits the enzymatic activity of EGFR. In some embodiments, an EGFR inhibitor inhibits a function of EGFR. In some embodiments, an EGFR inhibitor inhibits a protein-protein interaction of EGFR. In some embodiments, an EGFR inhibitor inhibits the normal localization of EGFR. In some embodiments, an EGFR inhibitor inhibits ligand binding to EGFR. In some embodiments, an EGFR inhibitor inhibits the binding of EGF to EGFR. In some embodiments, an EGFR inhibitor induces an inactive conformation of EGFR. In some embodiments, an EGFR inhibitor increases degradation of EGFR. In some embodiments, an EGFR inhibitor inhibits posttranslational modification of EGFR. An “EGFR inhibitor resistant” cancer, or specific form of cancer (e.g. lung cancer, non-small cell lung cancer, pancreatic cancer, breast cancer, colon cancer, esophageal cancer, thyroid cancer, liver cancer, glioblastoma, or astrocytoma-glioblastoma) is a cancer that is treated less effectively by one or more EGFR inhibitors than a non-EGFR inhibitor resistant cancer, wherein the comparison of the effectiveness of the resistant and non-resistant cancer is to the same inhibitor(s). EGFR inhibitors include gefitinib, erlotinib, cetuximab, lapatinib, panitumumab, vandetanib, afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626, zalutumumab, nimotuzumab, matuzumab, AP26113, and CO-1686. Other EGFR inhibitors are well known in the art and determining that such EGFR inhibitors may be examples of “EGFR inhibitors” is within the skill of a person of ordinary skill in the art. The terms “EGFR TKI” and “EGFR inhibitor” are used interchangeably and are equivalent.

As used herein, the term “AXL inhibitor” refers a composition (e.g. compound, antibody, drug, nucleic acid, siRNA, RNAi, protein, modulator) that decreases the activity of AXL, function of AXL, level of activity of AXL, level of function of AXL, level (e.g. amount) of AXL (e.g. protein, RNA), level of AXL in a particular physical state (e.g. phosphorylated AXL), expression of AXL, activity of GAS6, function of GAS6, level of activity of GAS6, level of function of GAS6, level (e.g. amount) of GAS6 (e.g. protein, RNA), level of GAS6 in a particular physical state, expression of GAS6, or activity, level (e.g. amount), or function of another AXL ligand. In some embodiments, a mutant AXL inhibitor is a compound that decreases the activity or function or level of activity or level of function of mutant AXL or level of mutant AXL or level of mutant AXL in a particular physical state. In some embodiments an AXL inhibitor is a compound that decreases the activity or function or level of activity or level of function of GAS6 or level of GAS6 or level of GAS6 in a particular physical state. In some embodiments, a mutant AXL inhibitor is a compound that decreases the activity or function or level of activity or level of function of mutant GAS6 or level of mutant GAS6 or level of mutant GAS6 in a particular physical state. AXL inhibitors include BGB324, amuvatinib/MP-470, foretinib/XL880, BMS-777607, SGI-7079, bosutinib, crizotinib, YW327.6S2, AD57, AD80, AD81, and AXL binding antibodies, Axl-Fc fusion protein. Other AXL inhibitors are well known in the art and determining that such AXL inhibitors may be examples of “AXL inhibitors” is within the skill of a person of ordinary skill in the art.

As used herein, the term “marker” refers to any biochemical marker, serological marker, genetic marker, or other clinical characteristic that can be used to diagnose or provide a prognosis for a cancer that expresses (e.g. overexpresses) AXL or GAS6 according to the methods of the present invention. Preferably, the marker is an AXL or GAS6 protein or nucleic acid marker. A marker may also refer to a characteristic (e.g. AXL or GAS6) that may be detected in the methods described herein.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., lung cancer, non-small cell lung cancer, pancreatic cancer, breast cancer, colon cancer, esophageal cancer, thyroid cancer, liver cancer, glioblastoma, astrocytoma-glioblastoma, EGFR inhibitor resistant forms thereof, etc.), the size and type of the tumor (e.g., solid or suspended, blood or ascites), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means parenteral administration, oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The term “administer (or administering) an AXL inhibitor” means administering an inhibitor (e.g. compound) that inhibits the activity or level (e.g. amount) of AXL and/or GAS6 or level of a signaling pathway of AXL and/or GAS6 (e.g. an AXL inhibitor) to a subject. Administration may include, without being limited by mechanism, allowing sufficient time for the AXL inhibitor to reduce the activity of AXL or GAS6 proteins or for the AXL inhibitor to reduce one or more symptoms (e.g. EGFR inhibitor resistance) of a disease (e.g. cancer, wherein the AXL inhibitor may overcome EGFR inhibitor resistance, arrest the cell cycle, slow the cell cycle, reduce DNA replication, reduce cell replication, reduce cell growth, reduce metastasis, or cause cell death).

The term “associated” or “associated with” in the context of a substance or substance activity or function associated with a disease (e.g. a protein associated disease, a cancer associated with aberrant AXL activity, AXL associated cancer, mutant AXL associated cancer, activated AXL associated cancer, mutant AXL associated cancer, a cancer associated with aberrant GAS6 activity, GAS6 associated cancer, mutant GAS6 associated cancer, activated GAS6 associated cancer, mutant GAS6 associated cancer) means that the disease (e.g. cancer) is caused by (in whole or in part), or a symptom of the disease is caused by (in whole or in part) the substance or substance activity or function. For example, a cancer associated with aberrant AXL activity or function may be a cancer that results (entirely or partially) from aberrant AXL activity or function (e.g. enzyme activity, protein-protein interaction, signaling pathway) or a cancer wherein a particular symptom of the disease is caused (entirely or partially) by aberrant AXL activity or function (e.g. EGFR inhibitor resistance). As used herein, what is described as being associated with a disease, if a causative agent, could be a target for treatment of the disease. For example, a cancer associated with aberrant AXL activity or function or an AXL associated cancer, may be treated with an AXL modulator or AXL inhibitor, in the instance where increased AXL activity or function (e.g. signaling pathway activity) causes the cancer. For example, a cancer associated with increased AXL may be a cancer that a subject with increased AXL is at higher risk of developing as compared to a subject without increased AXL (e.g. EGFR inhibitor resistant cancer).

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propogated to other signaling pathway components. For example, binding of AXL with a compound as described herein may result in a change in one or more protein-protein interactions of the AXL, resulting in changes in cell growth, proliferation, survival, or EGFR inhibitor resistance.

An amino acid residue in a protein “corresponds” to a given residue when it occupies the same essential structural position within the protein as the given residue. For example, a selected residue in a selected protein corresponds to Thr 790 of Human EGFR when the selected residue occupies the same essential spatial or other structural relationship as Thr 790 in Human EGFR. In some embodiments, where a selected protein is aligned for maximum homology with the Human EGFR protein, the position in the aligned selected protein aligning with Thr 790 is said to correspond to Thr 790. Instead of a primary sequence alignment, a three dimensional structural alignment can also be used, e.g., where the structure of the selected protein is aligned for maximum correspondence with the Human EGFR protein and the overall structures compared. In this case, an amino acid that occupies the same essential position as Thr 790 in the structural model is said to correspond to the Thr 790 residue.

The term “level of AXL” or “AXL level” refers to an amount of AXL nucleic acid, protein, or activity. In some embodiments, the level refers to the amount of an AXL nucleic acid, or fragment thereof. In some embodiments, the level refers to the amount of an AXL protein or fragment thereof. In some embodiments, the level refers to the amount of AXL activity (e.g. kinase activity, signaling pathway activity involving AXL, protein-protein binding). In some embodiments a level may refer to a subpopulation of AXL (e.g. phosphorylated, bound to ligand, localized). In some embodiment the level refers to the amount of AXL RNA. In some embodiments, the level refers to the amount of AXL DNA (e.g. copy number of the gene). In some embodiments, the level refers to the amount of a mutant AXL nucleic acid, protein, or activity.

The term “level of GAS6” or “GAS6 level” refers to an amount of GAS6 nucleic acid, protein, or activity. In some embodiments, the level refers to the amount of a GAS6 nucleic acid, or fragment thereof. In some embodiments, the level refers to the amount of a GAS6 protein or fragment thereof. In some embodiments, the level refers to the amount of GAS6 activity (e.g. kinase activity, signaling pathway activity involving GAS6, protein-protein binding). In some embodiments a level may refer to a subpopulation of GAS6 (e.g. phosphorylated, bound to ligand, localized). In some embodiment the level refers to the amount of GAS6 RNA. In some embodiments, the level refers to the amount of GAS6 DNA (e.g. copy number of the gene). In some embodiments, the level refers to the amount of a mutant GAS6 nucleic acid, protein, or activity.

The term “detectable AXL-binding agent” as used herein refers to a composition capable of binding an AXL nucleic acid, or fragment thereof, or protein, or fragment thereof, wherein the complex (e.g. including AXL and the detectable AXL-binding agent) is capable of being detected. Some examples include a nucleic acid that is capable of binding to AXL, or fragment thereof, wherein the complex of the detectable AXL-binding agent (e.g. nucleic acid) and the AXL nucleic acid can be detected by polymerase chain reaction resulting in increased nucleic acids derived from the complex (e.g. amplification of at least a portion of the AXL nucleic acid); an antibody that binds AXL, which is labeled with a detectable moiety or is capable of being labeled with a detectable moiety by binding of a secondary antibody conjugated to a detectable moiety to the AXL antibody.

The term “detectable GAS6-binding agent” as used herein refers to a composition capable of binding an GAS6 nucleic acid, or fragment thereof, or protein, or fragment thereof, wherein the complex (e.g. including GAS6 and the detectable GAS6-binding agent) is capable of being detected. Some examples include a nucleic acid that is capable of binding to GAS6, or fragment thereof, wherein the complex of the detectable GAS6-binding agent (e.g. nucleic acid) and the GAS6 nucleic acid can be detected by polymerase chain reaction resulting in increased nucleic acids derived from the complex (e.g. amplification of at least a portion of the GAS6 nucleic acid); an antibody that binds GAS6, which is labeled with a detectable moiety or is capable of being labeled with a detectable moiety by binding of a secondary antibody conjugated to a detectable moiety to the GAS6 antibody.

The term “diagnosis” refers to a relative probability that a disease (e.g. cancer, EGFR inhibitor resistant cancer, or other disease) is present in the subject. Similarly, the term “prognosis” refers to a relative probability that a certain future outcome may occur in the subject with respect to a disease state. For example, in the context of the present invention, prognosis can refer to the likelihood that an individual will develop a disease (e.g. cancer, EGFR inhibitor resistant cancer, or other disease), or the likely severity of the disease (e.g., duration of disease). The terms are not intended to be absolute, as will be appreciated by any one of skill in the field of medical diagnostics.

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. In certain embodiments. the nucleic acids herein contain phosphodiester bonds. In other embodiments, nucleic acid analogs are included that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A particular nucleic acid sequence also encompasses “splice variants.” Similarly, a particular protein encoded by a nucleic acid encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Unless otherwise indicated, a particular nucleic acid sequence also implicitly encompasses conservatively modified variants thereof (e.g., degenerate codon substitutions), alleles, orthologs, SNPs (haplotypes), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 10 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

The phrase “selectively (or specifically) hybridizes to” refers to the binding, duplexing, or hybridizing of a molecule only to a particular nucleotide sequence with a higher affinity, e.g., under more stringent conditions, than to other nucleotide sequences (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

Twenty amino acids are commonly found in proteins. Those amino acids can be grouped into nine classes or groups based on the chemical properties of their side chains. Substitution of one amino acid residue for another within the same class or group is referred to herein as a “conservative” substitution. Conservative amino acid substitutions can frequently be made in a protein without significantly altering the conformation or function of the protein. Substitution of one amino acid residue for another from a different class or group is referred to herein as a “non-conservative” substitution. In contrast, non-conservative amino acid substitutions tend to modify conformation and function of a protein.

### Example of Amino Acid Classification

Small/Aliphatic residues: Gly, Ala, Val, Leu, Ile

### Cyclic Imino Acid: Pro

### Hydroxyl-containing Residues: Ser, Thr

### Acidic Residues Asp, Glu

### Amide Residues Asn, Gln

### Basic Residues Lys, Arg

### Imidazole Residue His

### Aromatic Residues Phe, Tyr, Tip

### Sulfur-containing Residues: Met, Cys

In some embodiments, the conservative amino acid substitution comprises substituting any of glycine (G), alanine (A), isoleucine (I), valine (V), and leucine (L) for any other of these aliphatic amino acids; serine (S) for threonine (T) and vice versa; aspartic acid (D) for glutamic acid (E) and vice versa; glutamine (Q) for asparagine (N) and vice versa; lysine (K) for arginine (R) and vice versa; phenylalanine (F), tyrosine (Y) and tryptophan (W) for any other of these aromatic amino acids; and methionine (M) for cysteine (C) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pKs of these two amino acid residues are not significant. Still other changes can be considered “conservative” in particular environments (see, e.g., BIOCHEMISTRY at pp. 13-15, 2nd ed. Lubert Stryer ed. (Stanford University); Henikoff et al., Proc. Nat'l Acad. Sci. USA (1992) 89:10915-10919; Lei et al., J. Biol. Chem. (1995) 270(20):11882-11886).

“Polypeptide,” “peptide,” and “protein” are used herein interchangeably and mean any peptide-linked chain of amino acids, regardless of length or post-translational modification. As noted below, the polypeptides described herein can be, e.g., wild-type proteins, biologically-active fragments of the wild-type proteins, or variants of the wild-type proteins or fragments. Variants, in accordance with the disclosure, can contain amino acid substitutions, deletions, or insertions. The substitutions can be conservative or non-conservative.

Following expression, the proteins (e.g. antibodies, antigen-binding fragments thereof, conjugates, antibody-conjugates) can be isolated. The term “purified” or “isolated” as applied to any of the proteins described herein (e.g., a conjugate described herein, antibody or antigen-binding fragment thereof described herein) refers to a polypeptide that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

A “label” or a “detectable moiety” is a composition detectable by spectroscopic, photochemical, biochemical, immunochemical, chemical, magnetic resonance imaging, or other physical means. For example, useful labels include ³²P, fluorescent dyes, electron-dense reagents, enzymes (e.g., as commonly used in an ELISA), biotin, digoxigenin, paramagnetic molecules, paramagnetic nanoparticles, Gadolinium, radioisotopes, radionuclides (e.g. carbon-11, nitrogen-13, oxygen-15, fluorine-18, rubidium-82), fluorodeoxyglucose (e.g. fluorine-18 labeled), gamma ray emitting radionuclides, positron-emitting radionuclide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. Detectable moieties also include any of the above compositions derivatized for binding to a targeting agent (e.g. antibody or antigen binding fragment). Any method known in the art for conjugating an antibody to the label may be employed, e.g., using methods described in Hermanson, Bioconjugate Techniques 1996, Academic Press, Inc., San Diego.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301. In some embodiments, the term “antibody” includes monoclonal antibodies or antigen-binding fragments thereof, chimerized or chimeric antibodies or antigen-binding fragments thereof, humanized antibodies or antigen-binding fragments thereof, deimmunized human antibodies or antigen-binding fragments thereof, fully human antibodies or antigen-binding fragments thereof, single chain antibodies, single chain Fv fragments (scFv), Fv, Fd fragments, Fab fragments, Fab′ fragments, F(ab′)₂ fragments, diabodies or antigen-binding fragments thereof, minibodies or antigen-binding fragments thereof, triabodies or antigen-binding fragments thereof, domain antibodies or antigen-binding fragments thereof, camelid antibodies or antigen-binding fragments thereof, dromedary antibodies or antigen-binding fragments thereof, or bispecific antibodies or antigen-binding fragments thereof.

“Single chain Fv (scFv)” or “single chain antibodies” refers to a protein wherein the V_{H} and the V_{L} regions of a scFv antibody comprise a single chain which is folded to create an antigen binding site similar to that found in two chain antibodies. Methods of making scFv antibodies have been described in e.g., Ward et al., Exp Hematol. (5):660-4 (1993); and Vaughan et al., Nat Biotechnol. 14(3):309-14 (1996). Single chain Fv (scFv) antibodies optionally include a peptide linker of no more than 50 amino acids, generally no more than 40 amino acids, preferably no more than 30 amino acids, and more preferably no more than 20 amino acids in length. In some embodiments, the peptide linker is a concatamer of the sequence Gly-Gly-Gly-Gly-Ser, e.g., 2, 3, 4, 5, or 6 such sequences. However, it is to be appreciated that some amino acid substitutions within the linker can be made. For example, a valine can be substituted for a glycine. Additional peptide linkers and their use are well-known in the art. See, e.g., Huston et al., Proc. Nat'l Acad. Sci. USA 8:5879 (1988); Bird et al., Science 242:4236 (1988); Glockshuber et al., Biochemistry 29:1362 (1990); U.S. Pat. No. 4,946,778, U.S. Pat. No. 5,132,405 and Stemmer et al., Biotechniques 14:256-265 (1993).

As used herein, “chimeric antibody” refers to an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass.

As used herein, “humanized antibody” refers to an immunoglobulin molecule in which CDRs from a donor antibody are grafted onto human framework sequences. Humanized antibodies may also comprise residues of donor origin in the framework sequences. The humanized antibody can also comprise at least a portion of a human immunoglobulin constant region. Humanized antibodies may also comprise residues which are found neither in the recipient antibody nor in the imported CDR or framework sequences. Humanization can be performed using methods known in the art (e.g., Jones et al., Nature 321:522-525; 1986; Riechmann et al., Nature 332:323-327, 1988; Verhoeyen et al., Science 239:1534-1536, 1988); Presta, Curr. Op. Struct. Biol. 2:593-596, 1992; U.S. Pat. No. 4,816,567), including techniques such as “superhumanizing” antibodies (Tan et al., J. Immunol. 169: 1119, 2002) and “resurfacing” (e.g., Staelens et al., Mol. Immunol. 43: 1243, 2006; and Roguska et al., Proc. Natl. Acad. Sci. USA 91: 969, 1994).

In some embodiments, de-immunized antibodies or antigen-binding fragments thereof are provided. De-immunized antibodies or antigen-binding fragments thereof are antibodies that have been modified so as to render the antibody or antigen-binding fragment thereof non-immunogenic, or less immunogenic, to a given species (e.g., to a human). De-immunization can be achieved by modifying the antibody or antigen-binding fragment thereof utilizing any of a variety of techniques known to those skilled in the art (see, e.g., PCT Publication Nos. WO 04/108158 and WO 00/34317).

The disclosure also provides camelid or dromedary antibodies (e.g., antibodies derived from Camelus bactrianus, Calelus dromaderius, or lama paccos). Such antibodies, unlike the typical two-chain (fragment) or four-chain (whole antibody) antibodies from most mammals, generally lack light chains. See U.S. Pat. No. 5,759,808; Stijlemans et al. (2004) J Biol Chem 279:1256-1261; Dumoulin et al. (2003) Nature 424:783-788; and Pleschberger et al. (2003) Bioconjugate Chem 14:440-448.

As used herein, “complementarity-determining region (CDR)” refers to one of the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, for example, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space. Thus, the position of the CDRs within the V region is relatively conserved between antibodies.

The amino acid sequences and positions of the CDRs and framework regions can be determined using various well known definitions in the art, e.g., Kabat, Chothia, international ImMunoGeneTics database (IMGT), and AbM (e.g., Johnson et al., supra; Chothia & Lesk, 1987, J. Mol. Biol. 196, 901-917; Chothia C. et al., 1989, Nature 342, 877-883; Chothia C. et al., 1992, J. Mol. Biol. 227, 799-817; Al-Lazikani et al., J. Mol. Biol. 1997, 273(4), Ruiz et al., Nucleic Acids Res., 28, 219-221 (2000); Lefranc, M.-P. Nucleic Acids Res. January 1; 29(1):207-9 (2001); MacCallum et al, J. Mol. Biol., 262 (5), 732-745 (1996); Martin et al, Proc. Natl. Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

An “siRNA” or “RNAi” refers to a nucleic acid that forms a double stranded RNA, which double stranded RNA has the ability to reduce or inhibit expression of a gene or target gene when the siRNA expressed in the same cell as the gene or target gene (see, e.g., Bass, Nature, 411, 428-429 (2001); Elbashir et al., Nature, 411, 494-498 (2001); WO 00/44895; WO 01/36646; WO 99/32619; WO 00/01846; WO 01/29058; WO 99/07409; and WO 00/44914). “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferably about preferably about 20-30 base nucleotides, preferably about 20-25 nucleotides in length, e.g., 20, 21, 22, 23, 24, 25, 26, 27, 28, 29, or 30 nucleotides in length.

“Silencing” or “downregulation” refers to a detectable decrease of transcription and/or translation of a target sequence, i.e., the sequence targeted by the RNAi, or a decrease in the amount or activity of the target sequence or protein in comparison to the normal level that is detected in the absence of the interfering RNA or other nucleic acid sequence. A detectable decrease can be as small as 5% or 10%, or as great as 80%, 90% or 100%. More typically, a detectable decrease ranges from 20%, 30%, 40%, 50%, 60%, or 70%.

Double stranded siRNA that corresponds to the AXL (e.g. nucleic acid (SEQ ID NOS:26 or 27) encoding the protein, or fragment thereof) or GAS6 (e.g. nucleic acid (SEQ ID NOS:29 or 30) encoding the protein, or fragment thereof) gene, or a fragment thereof, can be used to silence the transcription and/or translation of AXL or GAS6 by inducing degradation of AXL or GAS6 mRNA transcripts, and thus treat or prevent cancer (e.g. EGFR inhibitor resistant cancer) by preventing expression of AXL or GAS6. The siRNA is typically about 5 to about 100 nucleotides in length, more typically about 10 to about 50 nucleotides in length, most typically about 15 to about 30 nucleotides in length. A DNA molecule that transcribes dsRNA or siRNA (for instance, as a hairpin duplex) also provides RNAi. DNA molecules for transcribing dsRNA are disclosed in U.S. Pat. No. 6,573,099, and in U.S. Patent Application Publication Nos. 2002/0160393 and 2003/0027783, and Tuschl and Borkhardt, Molecular Interventions, 2:158 (2002).

siRNA can be delivered to the subject using any means known in the art, including by injection, inhalation, or oral ingestion of the siRNA. Another suitable delivery system for siRNA is a colloidal dispersion system such as, for example, macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. Nucleic acids, including RNA and DNA within liposomes and be delivered to cells in a biologically active form (Fraley, et al., Trends Biochem. Sci., 6:77, 1981). Liposomes can be targeted to specific cell types or tissues using any means known in the art.

Antisense oligonucleotides that specifically hybridize to nucleic acid sequences encoding AXL or GAS6 polypeptides can also be used to silence the transcription and/or translation of AXL or GAS6, and thus treat or prevent cancer (e.g. EGFR inhibitor resistant cancer).

Antisense nucleic acids are DNA or RNA molecules that are complementary to at least a portion of a specific mRNA molecule (see, e.g., Weintraub, Scientific American, 262:40 (1990)). Typically, synthetic antisense oligonucleotides are generally between 15 and 25 bases in length. Antisense nucleic acids may comprise naturally occurring nucleotides or modified nucleotides such as, e.g., phosphorothioate, methylphosphonate, and -anomeric sugar-phosphate, backbone-modified nucleotides.

In the cell, the antisense nucleic acids hybridize to the corresponding mRNA, forming a double-stranded molecule. The antisense nucleic acids, interfere with the translation of the mRNA, since the cell will not translate a mRNA that is double-stranded. Antisense oligomers of about 15 nucleotides are preferred, since they are easily synthesized and are less likely to cause problems than larger molecules when introduced into the target nucleotide mutant producing cell. The use of antisense methods to inhibit the in vitro translation of genes is well known in the art (Marcus-Sakura, Anal. Biochem., 172:289, (1988)). Less commonly, antisense molecules which bind directly to the DNA may be used.

Delivery of antisense polynucleotides specific for the AXL or GAS6 gene can be achieved using any means known in the art including, e.g., direct injection, inhalation, or ingestion of the polynucleotides. In addition, antisense polynucleotides can be delivered using a recombinant expression vector (e.g., a viral vector based on an adenovirus, a herpes virus, a vaccinia virus, or a retrovirus) or a colloidal dispersion system (e.g., liposomes) as described herein.

The terms “differential expression” or “differentially expressed” used in reference to the expression of a marker means an elevated level of expression of the marker or a lowered level of expression (e.g. transcription or translation) of the marker gene relative to a control that is indicative of an EGFR inhibitor resistant cancer or cell or indicative of a patient having an EGFR TKI resistant cancer (an “EGFR TKI resistant cancer patient”). “Target sequence” refers to a region within a target gene (e.g., marker gene) which a probe will identify, as known in the art. It is understood that one of skill in the art can, with only routine experimentation, design and use probes to identify specific markers as described herein (e.g. AXL (e.g. protein (SEQ ID NO:28) or fragment thereof and/or a nucleic acid (SEQ ID NOS:26 or 27) encoding the protein, or fragment thereof) or GAS6 (e.g. protein (SEQ ID NO:31) or fragment thereof and/or a nucleic acid (SEQ ID NOS:29 or 30) encoding the protein, or fragment thereof)). It is further understood that more than one probe may be designed to identify a specific nucleic acid or protein, for example a marker described herein (e.g. AXL (e.g. protein (SEQ ID NO:28) or fragment thereof and/or a nucleic acid (SEQ ID NOS:26 or 27) encoding the protein, or fragment thereof) or GAS6 (e.g. protein (SEQ ID NO:31) or fragment thereof and/or a nucleic acid (SEQ ID NOS:29 or 30) encoding the protein, or fragment thereof)). In embodiments, differential expression is an elevated level of expression of a marker (e.g. AXL (e.g. protein (SEQ ID NO:28) or fragment thereof and/or a nucleic acid (SEQ ID NOS:26 or 27) encoding the protein, or fragment thereof) or GAS6 (e.g. protein (SEQ ID NO:31) or fragment thereof and/or a nucleic acid (SEQ ID NOS:29 or 30) encoding the protein, or fragment thereof)).

The term “AXL” refers to the human AXL receptor tyrosine kinase (e.g. GenBank AAH32229.1, NM_{—}001699.4, NP_{—}001690.2, P30530 and homologs thereof). In embodiments, “AXL” refers to the protein (e.g. SEQ ID NO:28 and homologs thereof) and/or a nucleic acid (SEQ ID NOS:26-27) encoding the protein.

The term “GAS6” refers to the human growth-arrest-specific protein (e.g. GenBank AAA58494.1, NM_{—}000820.2, NP_{—}000811.1, Q14393 and homologs thereof). In embodiments, “GAS6” refers to the protein (e.g. SEQ ID NO:31 and homologs thereof) and/or a nucleic acid (SEQ ID NOS:29-30) encoding the protein.

The term “EGFR” refers to the epidermal growth factor receptor (e.g. NM_{—}005228.3, NP_{—}005219.2, P00533). In embodiments, “EGFR” refers to the protein (e.g. SEQ ID NO:34 and homologs thereof) and/or a nucleic acid (e.g SEQ ID NOS:32-33 and homologs thereof) encoding the protein.

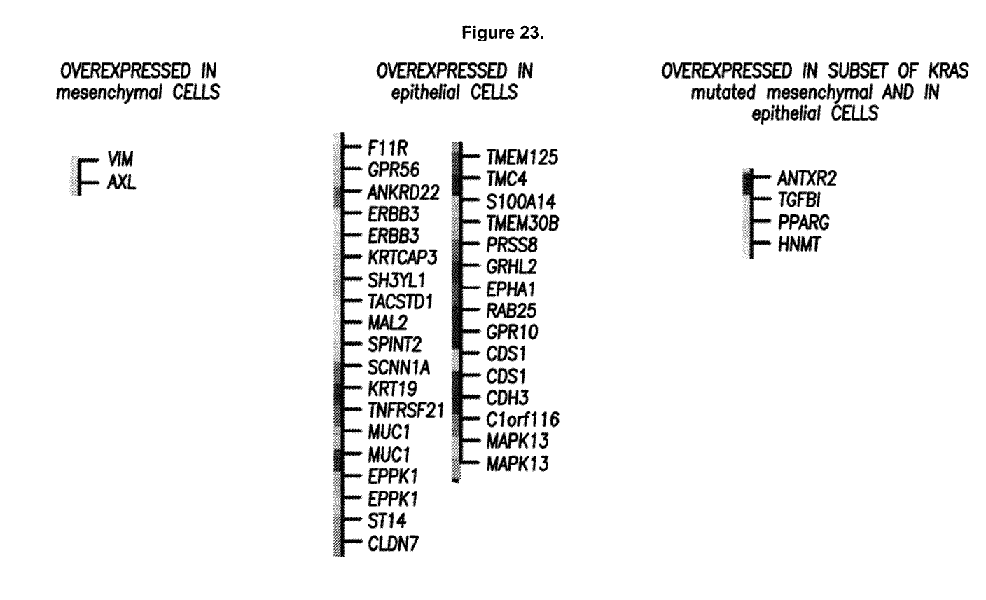

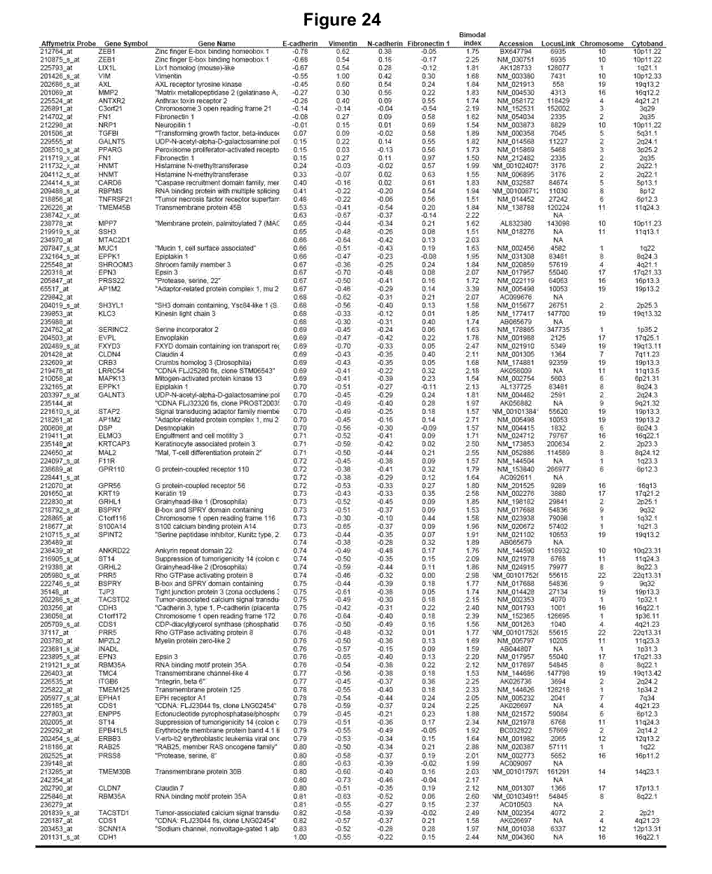

The term “EMT marker” refers to a marker (e.g. nucleic acid or protein) that is indicative of an epithelial-to-mesenchymal-transition (“EMT”), excluding AXL and GAS6. A collection of EMT markers may be referred to herein as an “EMT signature”. In embodiments, an EMT marker is a marker identified by gene symbol, gene name, or is a gene targeted by an Affymetrix Probe, all as identified in FIG. 23, FIG. 24, Table 6, 7, or 8; or one of the five markers 219789_at (NPR3), 219790_s_at (C5orf23), 212531_at (LCN2), 205760_s_at (OGG1), and 205301_s_at identified by Affymetrix Probe number or gene name where provided. See Byers et al. Clin. Cancer Res. 2013 January 1:19(1):279-90 and WO2012/135841.

The term “mesenchymal cancer” refers to a cancer associated with (e.g. derived from or composed of) cells identified as mesenchymal. A mesenchymal cell may be identified by determining the differential expression of EMT markers additionally optionally including AXL and/or GAS6 (e.g. one, more, or all of the EMT markers in FIG. 23, FIG. 24, Table 6, Table 7, Table 8, or the five markers 219789_at (NPR3), 219790_s_at (C5orf23), 212531_at (LCN2), 205760_s_at (OGG1), and 205301_s_at identified by Affymetrix Probe number or gene name where provided; one, more, or all of the 76 markers in Table 6 or FIG. 24; one, more, or all of the 35 markers in Table 7 or FIG. 23; one, more, or all of the 35 markers in Table 8 or FIG. 23; one, more, or all of the five markers 219789_at (NPR3), 219790_s_at (C5orf23), 212531_at (LCN2), 205760_s_at (OGG1), and 205301_s_at identified by Affymetrix Probe number or gene name where provided). See Byers et al. Clin. Cancer Res. 2013 January 1:19(1):279-90 and WO2012/135841.

### Methods of Treatment, Detection, and Identification

In an aspect is provided a method of treating epidermal growth factor receptor (EGFR) inhibitor resistant cancer. The method includes detecting an increased level of AXL or GAS6 in a patient sample relative to a control sample and administering a therapeutically effective amount of an AXL inhibitor or a GAS6 inhibitor to the patient.

In another aspect is provided a method of detecting AXL or GAS6 levels in a cancer patient. The method includes contacting a sample from the cancer patient with a detectable AXL-binding agent or detectable GAS6-binding agent, allowing the detectable AXL-binding agent or the detectable GAS6-binding agent to bind to AXL or GAS6, respectively, allowing the detectable AXL-binding agent and AXL to form an AXL complex or the GAS6-binding agent and GAS6 to form a GAS6 complex; and detecting the AXL complex or GAS6 complex.

In another aspect is provided a method of identifying an EGFR inhibitor resistant cancer patient including the steps of obtaining a sample from a plurality of cancer patients, detecting a level of AXL or a level of GAS6 in each of the samples, comparing the level of AXL or the level of GAS6 to a control, identifying at least one sample from the plurality of cancer patients having a level of AXL or a level of GAS6 greater than the control, and thereby identifying an EGFR inhibitor resistant cancer patient.

In another aspect is provided a method of treating EGFR inhibitor resistant cancer. The method includes detecting an increased level of AXL activity in a patient sample relative to a control sample and administering a therapeutically effective amount of an AXL inhibitor to the patient.

In another aspect is provided a method of identifying an EGFR inhibitor resistant cancer patient including the steps of obtaining samples from a plurality of cancer patients, detecting a level of AXL activity in each of the samples, comparing the level of AXL activity to a level of AXL activity in a control sample, identifying at least one sample having a level of AXL activity greater than the control, thereby identifying an EGFR inhibitor resistant cancer patient.

In another aspect is provided a method of detecting a level of AXL or GAS6 in a subject (e.g. a cancer patient), the method including: (i) obtaining a sample from a subject (e.g. cancer patient); and (ii) detecting a differential expression level of AXL or GAS6 in the sample relative to a control. In embodiments, the method further includes correlating the differential expression of AXL or GAS6 to a cancer (e.g. EGFR-activating mutation containing and/or EGFR inhibitor resistant cancer), as disclosed herein. Thus, the method may be used for diagnosing a subject with cancer (e.g. EGFR-activating mutation containing and/or EGFR inhibitor resistant cancer). In another aspect is provided a method of detecting an increased level of AXL or GAS6 in a subject (e.g. cancer patient), the method including: (i) obtaining a sample from a subject (e.g. cancer patient); and (ii) detecting an increased level of AXL or GAS6 in the sample relative to a control. In embodiments, the method further includes correlating the increased level of AXL or GAS6 to a cancer (e.g. EGFR-activating mutation containing and/or EGFR inhibitor resistant cancer), as disclosed herein. Thus, the method may be used for diagnosing a subject with cancer (e.g. EGFR-activating mutation containing and/or EGFR inhibitor resistant cancer).

In another aspect is provided a method of identifying an EGFR inhibitor resistant cancer patient including: (i) obtaining a sample from a subject (e.g. cancer patient); and (ii) detecting an increased level of AXL or GAS6 in the sample relative to a control.

In embodiments, the cancer patient has a cancer including cancer cells with an EGFR-activating mutation. In embodiments, the EGFR-activating mutation is an exon-19 deletion or an exon-21 point mutant. In embodiments, the method does not include detecting a level of an EMT marker, except AXL and/or GAS6. In embodiments, the method does not include detecting a level of an EMT marker, except AXL. In embodiments, the method does not include detecting a level of an EMT marker, other than AXL and/or GAS6. In embodiments, the method does not include detecting a level of an EMT marker, other than AXL. In embodiments, the detecting includes (a) contacting the sample with a detectable AXL-binding agent or detectable GAS6-binding agent; (b) allowing the detectable AXL-binding agent and AXL to form an AXL complex or the GAS6-binding agent and GAS6 to form a GAS6 complex; and (c) detecting the AXL complex or GAS6 complex. In embodiments, the detecting includes detecting a level of an AXL or GAS6 nucleic acid or fragment thereof. In embodiments, the detecting includes use of nucleic acid amplification, a gene array, a microarray, a macroarray, a DNA array, or a DNA chip. In embodiments, the detecting includes detecting a level of an AXL or GAS6 protein or fragment thereof. In embodiments, the detecting includes use of an antibody, flow cytometry, ELISA, mass spectroscopy, immunofluorescence, or fluorescence microscopy. In embodiments, the antibody is conjugated to a detectable moiety. In embodiments, the method includes detecting an increased level of AXL. In embodiments, the level of AXL is a level of AXL mRNA, AXL protein, or AXL kinase activity. In embodiments, the detectable AXL-binding agent or detectable GAS6-binding agent is a nucleic acid. In embodiments, the detectable AXL-binding agent or detectable GAS6-binding agent is a nucleic acid including a sequence identical to or complementary to a portion of SEQ ID NOS:26, 27, 29, or 30.

In embodiments, the cancer patient is an EGFR TKI resistant cancer patient. In embodiments, the cancer patient is an EGFR TKI resistant cancer patient, wherein the EGFR TKI resistant cancer patient has a cancer that is not a mesenchymal cancer. In embodiments, the EGFR TKI resistant cancer patient is resistant to gefitinib, erlotinib, cetuximab, lapatinib, panitumumab, vandetanib, afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626, zalutumumab, nimotuzumab, matuzumab, AP26113, or CO-1686. In embodiments, the EGFR TKI resistant cancer patient is resistant to erlotinib or gefitinib. In embodiments, the EGFR TKI resistant cancer patient has a cancer selected from the group consisting of lung cancer, pancreatic cancer, breast cancer, colon cancer, esophageal cancer, thyroid cancer, liver cancer, glioblastoma, and astrocytoma-glioblastoma. In embodiments, the lung cancer is non-small cell lung cancer (NSCLC). In embodiments, the cancer is metastatic cancer. In embodiments, the cancer includes EGFR having an exon-19 deletion or an exon-21 point mutant. In embodiments, the cancer is non-small cell lung cancer including EGFR having an exon-19 deletion or an exon-21 point mutant; and further wherein the cancer is erlotinib resistant or gefitinib resistant relative to a control. In embodiments, the detecting includes comparing the increased level of AXL or GAS6 to a control; identifying at least one sample from a plurality of cancer patients having a level of AXL or a level of GAS6 greater than the control; and thereby identifying an EGFR inhibitor resistant cancer patient. In embodiments, a higher level of AXL or GAS6 in the sample relative to the control is indicative of an EGFR inhibitor resistant cancer (e.g. further having an EGFR-activating mutation).

It has been discovered that certain genes are markers of EGFR inhibitor resistant cancers (e.g. AXL (e.g. protein (SEQ ID NO:28) and/or a nucleic acid (SEQ ID NOS:26 or 27) encoding the protein) or GAS6 (e.g. protein (SEQ ID NO:31) and/or a nucleic acid (SEQ ID NOS:29 or 30) encoding the protein)). Thus, by detecting the level of expression of a marker within a cancer patient and comparing the level of expression of the maker to a control, EGFR TKI resistant cancer patients may be identified. In some embodiments, the level of a plurality (e.g. a panel) of markers are detected and compared to the level of expression of the makers in a control to identify EGFR TKI resistant cancer patients. In some embodiments, the control may be approximately the average amount of expression of the marker in humans or humans without cancer, or humans who are not EGFR TKI resistant cancer patients. In other embodiments, the control is a detected level of expression of a control gene in the EGFR TKI resistant cancer patient.

In some embodiments, the methods include detecting an increased level of AXL. An increased level of GAS6 may also be detected. The increased level of GAS6 or AXL may be an increased level of protein (e.g. AXL protein, GAS6 protein, or protein fragments thereof).

In other embodiments, the increased levels of an AXL or GAS6 may be an increased level of nucleic acid (e.g. AXL nucleic acid, GAS6 nucleic acid, or a nucleic acid fragment thereof). The nucleic acid detected may be mRNA.

In other embodiments, increased levels of AXL (e.g. AXL activity) may be detected. In embodiments, an increased level of AXL is detected and increased levels of GAS6 are not detected.

In some embodiments, the methods further include administering a combined therapeutically effective amount of an EGFR inhibitor and an AXL inhibitor. In some embodiments, the amount of EGFR inhibitor administered is less than a therapeutically effective amount of the EGFR inhibitor that would be administered in the absence of the AXL inhibitor. In some embodiments, the EGFR inhibitor is selected from the group consisting of gefitinib, erlotinib, cetuximab, lapatinib, panitumumab, vandetanib, afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626, zalutumumab, nimotuzumab, matuzumab, AP26113, and CO-1686. In some embodiments, the EGFR inhibitor is erlotinib. In some embodiments, the EGFR inhibitor is gefitinib. In some embodiments, the EGFR inhibitor is lapatinib. In some embodiments, the EGFR inhibitor is cetuximab. In some embodiments, the EGFR inhibitor is panitumumab. In some embodiments, the EGFR inhibitor is vandetanib. In some embodiments, the EGFR inhibitor is afatinib. In some embodiments, the EGFR inhibitor is desmethyl erlotinib. In some embodiments, the EGFR inhibitor is CO-1686. In some embodiments, the EGFR inhibitor is AP26113.

In some embodiments, the AXL inhibitor is selected from the group consisting of BGB324, amuvatinib, foretinib, BMS-777607, SGI-7079, bosutinib, crizotinib, YW327.6S2, AD57, AD80, AD81, AXL binding antibody, and Axl-Fc fusion protein. In some embodiments, the AXL inhibitor is not an antibody. In some embodiments, the AXL inhibitor is not an Axl-Fc fusion protein

In some embodiments, the methods include detecting a level of an AXL or GAS6 nucleic acid or fragment thereof, including homologs thereof. In some embodiments, the nucleic acid is an AXL RNA, or a fragment thereof (e.g. about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% of the full length nucleic acid sequence of AXL, for example human AXL represented by GenBank AAH32229.1 or NM_{—}001699.4 or SEQ ID NO:26 or SEQ ID NO:27). In some embodiments, the nucleic acid is a GAS6 RNA, or a fragment thereof, including homologs thereof (e.g. about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% of the full length nucleic acid sequence of GAS6, for example human GAS6 represented by GenBank AAA58494.1 or NM_{—}000820.2 or SEQ ID NO:29 or SEQ ID NO:30).

In some embodiments, the detecting includes use of nucleic acid amplification, polymerase chain reaction (PCR), real time PCR/quantitative real time PCR, real time reverse transcription PCR, nucleic acid intercalating dye (e.g. SYBR green) with real time PCR or real time reverse transcription PCR, oligonucleotide probes (e.g. that specifically hybridize to AXL or GAS6) with fluorophore-quencher pair label (e.g. FRET) and real time PCR or real time reverse transcription PCR, a gene array, a microarray, a macroarray, a DNA array, tiling array, northern blot, serial analysis of gene expression (SAGE), Next-generation sequencing (NGS) (e.g. RNA-Seq), Whole Transcriptome Shotgun Sequencing (WTSS), deep sequencing, or a DNA chip. In some embodiments, the detecting includes detecting a level of an AXL or GAS6 protein or fragment thereof. In some embodiments, the protein is an AXL protein, or a fragment thereof, including homologs thereof (e.g. about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% of the full length protein sequence of AXL, for example human AXL represented by NP_{—}001690.2 or SEQ ID NO:28). In some embodiments, the protein is a GAS6 protein, or a fragment thereof, including homologs thereof (e.g. about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% of the full length protein sequence of GAS6, for example human GAS6 represented by NP_{—}000811.1 or SEQ ID NO:31).

In some embodiments, the detecting includes use of an antibody, western blot, protein chip, immunohistochemistry, flow cytometry, ELISA, mass spectroscopy (e.g. LC-MS, MALDI-MS, MALDI-MS/MS, ESI-MS, ESI-MS/MS, tandem mass spectrometry), immunofluorescence, chromatography (e.g. HPLC, FLPC, reverse phase, LC-MS), spectrophotometric assays, UV spectroscopy, visible spectroscopy, gel electrophoresis, two-dimensional gel electrophoresis, N-terminal labeling, isotope-coded affinity tags (ICAT), isobaric tags (e.g. tandem mass tags, isobaric tags for relative and absolute quantitation (iTRAQ)), stable isotope labeling, metal-coded tags (MeCATs), selected reaction monitoring (SRM), or fluorescence microscopy.

In some embodiments, the antibody used in detecting AXL, GAS6, or a fragment thereof of either, is selected from the group consisting of a monoclonal antibody or antigen-binding fragment thereof, chimerized or chimeric antibody or antigen-binding fragment thereof, humanized antibody or antigen-binding fragment thereof, deimmunized human antibody or antigen-binding fragment thereof, fully human antibody or antigen-binding fragment thereof, single chain antibody, single chain Fv fragment (scFv), Fv, Fd fragment, Fab fragment, Fab′ fragment, F(ab′)₂ fragment, diabody or antigen-binding fragment thereof, minibody or antigen-binding fragment thereof, triabody or antigen-binding fragment thereof, domain antibody or antigen-binding fragment thereof, camelid antibody or antigen-binding fragment thereof, dromedary antibody or antigen-binding fragment thereof, and a bispecific antibody or antigen-binding fragment thereof. In some embodiments, the antibody is conjugated to a detectable moiety.

In some embodiments, the detectable moiety includes a fluorophore. In some embodiments, the detectable moiety includes an enzyme. In some embodiments, the detectable moiety includes biotin or streptavidin. In some embodiments, the increased level of AXL or GAS6 relative to the control is indicative of an EGFR inhibitor resistant cancer. The increased level of AXL or GAS6 relative to the control may be, for example, about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000 fold greater. The increased level of AXL or GAS6 relative to the control may be, for example, about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000 fold.

In certain embodiments, the method described herein for detecting the level of expression of a marker is an in vitro method. In some embodiments, detection is conducted in vitro (e.g. on a biological sample derived from a cancer patient).

The expression levels of the marker may be measured using any appropriate method. In some embodiments, the amount of RNA expressed by the marker is measured. The amount of RNA expressed may be assessed, for example, using nucleic acid probes with marker coding sequences or using quantitative PCR techniques. For example, a nucleic acid array forming a probe set may be used to detect RNA expressed by the marker gene. The RNA expressed by the marker gene may be transcribed to cDNA (and in some cases to cRNA) and then queried with a gene chip array using methods known in the art. Thus, in some embodiments the marker may also be a gene including a nucleic acid sequence having at least 90%, 91%, 92%, 93%, 94%, 95%, 96%, 97%, 98%, 99%, or 100% identity over at least a 10 or 20 nucleotide continuous region (i.e. sequence) within a marker gene (e.g. AXL (e.g. nucleic acid (SEQ ID NOS:26 or 27) encoding the protein or fragment thereof) or GAS6 (e.g. nucleic acid (SEQ ID NOS:29 or 30) encoding the protein, or fragment thereof)). For example, the continuous region may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length. The term “designed to interrogate” in the context of target genes, marker genes and probes refers to a probe having sufficient primary sequence complementarity to a target to detectably bind the target, as well known in the art.

In some embodiments, the marker includes the nucleic acid sequence of AXL (e.g. nucleic acid (SEQ ID NOS:26 or 27)) or GAS6 (e.g. nucleic acid (SEQ ID NOS:29 or 30)) or a fragment thereof (e.g. about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% of the full length nucleic acid sequence or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% of the full length nucleic acid sequence).

The comparison of the marker expression levels with a control may be accomplished by determining whether the marker is expressed in the EGFR TKI resistant cancer patient at an elevated level or a lowered level (i.e. detecting differential expression). The elevated level of AXL and/or GAS6 is indicative of EGFR TKI resistant cancer (e.g. that may be treated with an AXL inhibitor or GAS6 inhibitor).

The control may be any appropriate standard known in the art. In some embodiments, the control is approximately the average amount of expression of the marker gene in humans, humans without cancer, or humans with EGFR TKI sensitive cancer.

In embodiments, the control is a detected level of expression of a standard control gene in the CIS patient (“standard control”). As used herein, a standard control gene is a human gene that is expressed at approximately constant levels thereby providing a baseline reading of gene expression for an individual. The standard control gene may also be referred to herein and in the art as a housekeeping gene. In some embodiments, the standard control gene is GAPDH or 18s ribosomal subunit.

The elevated level of expression of the marker or the lowered level of expression of the marker may be determined by calculating the ratio of the level of expression of the marker to the level of expression of a standard control.

In another aspect, there is provided an in vitro method for determining whether a patient is an EGFR TKI resistant cancer patient. The method includes isolating mRNA from the patient, thereby providing an in vitro nucleic acid sample. Optionally, the method further includes subjecting the in vitro nucleic acid sample to polymerase chain reaction under conditions suitable to amplify nucleic acid within the in vitro nucleic acid sample. The in vitro nucleic acid sample is contacted with a microarray, the microarray having a plurality of probes designed to interrogate specific marker genes. The level of nucleic acid duplex formation is determined between the in vitro nucleic acid sample and the microarray, thereby providing the expression level of nucleic acid present in the in vitro nucleic acid sample. The expression level of nucleic acid is then compared to the expression level of a control or standard control. A differential expression of the marker gene relative to said control or standard control indicates that the patient is an EGFR TKI resistant cancer patient (e.g. having an EGFR-activating mutation) (e.g. that may be treated with an AXL inhibitor or GAS6 inhibitor).

In another aspect, a kit is provided for use in identifying a patient who is an EGFR TKI resistant cancer patient (e.g. having an EGFR-activating mutation) (e.g. that may be treated with an AXL inhibitor or GAS6 inhibitor). The kit includes a nucleic acid sequence having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity over at least a 10 nucleotide continuous region (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length) of AXL (e.g. nucleic acid (SEQ ID NOS:26 or 27)) or GAS6 (e.g nucleic acid (SEQ ID NOS:29 or 30); or a nucleic acid complimentary to the nucleic acids set forth above. In some embodiments, the kit also includes an electronic device or computer software capable of comparing a marker gene expression level from the patient to a control thereby indicating whether the patient is an EGFR TKI resistant cancer patient (e.g. that may be treated with an AXL inhibitor or GAS6 inhibitor). In some embodiments, the kit contains a plurality (e.g., 2, 3, 4, 5, 6, 7, 8, 9, 10, 11, 12, 13, 14, 15, 16, 17, 18, 19 or 20) of nucleic acid sequences having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity over at least a 10 nucleotide continuous region (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length) of AXL (e.g. nucleic acid (SEQ ID NOS:26 or 27)) or GAS6 (e.g nucleic acid (SEQ ID NOS:29 or 30), or complement thereof.

In some embodiments, the nucleic acid provided in the kit above may be a probe nucleic acid for use in a PCR technique, such as quantitative PCR, to assess the expression of a given marker gene. In some embodiments, the nucleic acid sequence has 100% identity with a continuous nucleic acid region (i.e. sequence) within AXL (e.g. nucleic acid (SEQ ID NOS:26 or 27)) or GAS6 (e.g nucleic acid (SEQ ID NOS:29 or 30)), or is complimentary thereto.

The nucleic acid provided in the kit may also hybridize under stringent conditions (or moderately stringent conditions) to a nucleic acid sequence within AXL (e.g. nucleic acid (SEQ ID NOS:26 or 27)) or GAS6 (e.g nucleic acid (SEQ ID NOS:29 or 30). The nucleic acid provided in the kit may also be perfectly complimentary to a nucleic acid sequence within AXL (e.g. nucleic acid (SEQ ID NOS:26 or 27)) or GAS6 (e.g nucleic acid (SEQ ID NOS:29 or 30).

In some embodiments, the cancer is selected from the group consisting of lung cancer, pancreatic cancer, breast cancer, colon cancer, esophageal cancer, thyroid cancer, liver cancer, glioblastoma, and astrocytoma-glioblastoma. In some embodiments, the lung cancer is non-small cell lung cancer (NSCLC). In some embodiments, the EGRF inhibitor resistant cancer is selected from the group consisting of lung cancer, non-small cell lung cancer, pancreatic cancer, breast cancer, colon cancer, esophageal cancer, thyroid cancer, liver cancer, glioblastoma, and astrocytoma-glioblastoma. In some embodiments, the non-small cell lung cancer (NSCLC) is an adenocarcinoma. In some embodiments, the non-small cell lung cancer (NSCLC) is a squamous cell carcinoma. In some embodiments, the non-small cell lung cancer (NSCLC) is a large cell carcinoma. In some embodiments, the non-small cell lung cancer (NSCLC) is a pleomorphic carcinoma. In some embodiments, the non-small cell lung cancer (NSCLC) is a carcinoid tumor. In some embodiments, the non-small cell lung cancer (NSCLC) is a salivary gland carcinoma. In some embodiments, the non-small cell lung cancer (NSCLC) is a carcinoma.

In some embodiments, the EGFR inhibitor resistant cancer is resistant to an EGFR inhibitor selected from the group consisting of gefitinib, erlotinib, cetuximab, lapatinib, panitumumab, vandetanib, afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626, zalutumumab, nimotuzumab, matuzumab, AP26113, and CO-1686. In some embodiments, the EGFR inhibitor is erlotinib. In some embodiments, the EGFR inhibitor is gefitinib. In some embodiments, the EGFR inhibitor is lapatinib. In some embodiments, the EGFR inhibitor is cetuximab. In some embodiments, the EGFR inhibitor is panitumumab. In some embodiments, the EGFR inhibitor is vandetanib. In some embodiments, the EGFR inhibitor is afatinib. In some embodiments, the EGFR inhibitor is desmethyl erlotinib. In some embodiments, the EGFR inhibitor is CO-1686. In some embodiments, the EGFR inhibitor is AP26113. In some embodiments, the EGFR inhibitor resistant cancer is resistant to erlotinib and gefitinib.

In some embodiments of the methods (e.g. method of treating, diagnosing, identifying, detecting), the method includes determining the presence of an EGFR mutation (e.g. activating mutation) in the sample (e.g. patient sample). In some embodiments of the methods, the patient and/or patient sample does not include an EGFR mutation. In some embodiments of the methods, the patient and/or patient sample does include an EGFR mutation.

In embodiments of the methods (e.g. method of treating, diagnosing, identifying, or detecting), the method includes a non-wildtype EGFR. In embodiments of the methods (e.g. method of treating, diagnosing, identifying, or detecting), the method includes a mutant EGFR. In embodiments of the methods (e.g. method of treating, diagnosing, identifying, or detecting), the method includes an EGFR including an activating mutation (“EGFR-activating mutation”) (e.g. a kinase domain activating mutation (e.g. deletion in exon-19, point mutation in exon 21, mutation in exon-18, or mutation in exon-20)). In embodiments of the methods (e.g. method of treating, diagnosing, identifying, or detecting), the method includes an EGFR including an EGFR-activating mutation (e.g. deletion in exon-19, point mutation in exon 21, mutation in exon-18, or mutation in exon-20) and includes a cancer, tumor, cell, or patient, that is EGFR inhibitor resistant (e.g. relative to a control).

In embodiments of the methods (e.g. method of treating, diagnosing, identifying, or detecting), the method includes a deletion mutant in exon-19 of EGFR (human EGFR numbering). In embodiments of the methods (e.g. method of treating, diagnosing, identifying, or detecting), the method includes an E746-A750 exon-19 deletion EGFR mutant (human EGFR numbering). In embodiments of the methods (e.g. method of treating, diagnosing, identifying, or detecting), the method includes an exon-19 deletion EGFR mutant (e.g. deletion (del) of L747-T751, E746-A750, E746-S752, L747-K754, I744-A750, L747-A750; deletion of amino acids 746-753, 747-753, 748-753, 749-753, 750-753, 751-753, 752-753, 746-752, 747-752, 748-752, 749-752, 750-752, 751-752, 746-751, 747-751, 748-751, 749-751, 750-751, 746-750, 747-750, 748-750, 749-750, 746-749, 747-749, 748-759, 746-748, 747-748, 746-747, 746, 747, 748, 749, 750, 751, 752, or 753, all using human EGFR numbering). In embodiments of the methods (e.g. method of treating, diagnosing, identifying, or detecting), the method includes a patient, cancer, tumor, or cell that has become EGFR inhibitor resistant following contact with an EGFR inhibitor.

In embodiments of the methods (e.g. method of treating, diagnosing, identifying, or detecting), the method includes an exon-21 point mutant EGFR. In embodiments of the methods (e.g. method of treating, diagnosing, identifying, or detecting), the method includes an exon-21 L858R point mutant EGFR (human numbering). In embodiments of the methods (e.g. method of treating, diagnosing, identifying, or detecting), the method includes an exon-21 point mutant EGFR (e.g. N826S, T847I, H850N, V851A, I853T, L858R, L861Q, A864T, E866K, or G873E, all using human EGFR numbering). In embodiments of the methods (e.g. method of treating, diagnosing, identifying, or detecting), the method includes a patient, cancer, tumor, or cell that tests positive for an activating EGFR mutation (e.g. on a cobas EGFR mutation test). In embodiments of the methods (e.g. method of treating, diagnosing, identifying, or detecting), the method includes a patient, cancer, tumor, or cell that tests negative for an activating EGFR mutation (e.g. on a cobas EGFR mutation test).

In some embodiments of the methods (e.g. method of treating, diagnosing, identifying, detecting), the method includes determining the presence of an EGFR T790M mutation in the sample (e.g. patient sample) and/or patient. In some embodiments of the methods, the patient and/or patient sample does not include an EGFR T790M mutation. In some embodiments of the methods, the patient and/or patient sample does include an EGFR T790M mutation.

In some embodiments of the methods (e.g. method of treating, diagnosing, identifying, detecting), the method includes determining the presence of an increased (e.g. higher, greater) level of MET in the sample (e.g. patient sample) and/or patient relative to a control sample or control. In some embodiments of the methods, the patient and/or patient sample does not include an increased (e.g. higher, greater) level of MET relative to a control. In some embodiments of the methods, the patient and/or patient sample does include an increased (e.g. higher, greater) level of MET relative to a control.

In some embodiments of the methods (e.g. method of treating, diagnosing, identifying, detecting), the method includes determining the presence of an endothelial to mesenchymal transition or transformation (EMT) in the sample (e.g. patient sample) and/or patient relative to a control sample or control. In some embodiments of the methods, the patient and/or patient sample does not include the presence of an EMT relative to a control. In some embodiments of the methods, the patient and/or patient sample does include the presence of an EMT relative to a control. In some embodiments, the EMT includes loss of cell adhesion, increased cell motility, and/or reduction in the levels of E-cadherin.

### Pharmaceutical Compositions

In another aspect is provided a pharmaceutical composition including a combined therapeutically effective amount of an AXL inhibitor and an EGFR inhibitor.

In some embodiments, the AXL inhibitor is selected from the group consisting of BGB324, amuvatinib, foretinib, BMS-777607, SGI-7079, bosutinib, crizotinib, YW327.6S2, AD57, AD80, AD81, AXL binding antibody, and Axl-Fc fusion protein. In some embodiments, the AXL inhibitor is not an antibody or an Axl-Fc fusion protein. In some embodiments, the EGFR inhibitor is selected from the group consisting of gefitinib, erlotinib, cetuximab, lapatinib, panitumumab, vandetanib, afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626, zalutumumab, nimotuzumab, matuzumab, AP26113, and CO-1686. In some embodiments, the EGFR inhibitor is erlotinib. In some embodiments, the EGFR inhibitor is gefitinib. In some embodiments, the EGFR inhibitor is lapatinib. In some embodiments, the EGFR inhibitor is cetuximab. In some embodiments, the EGFR inhibitor is panitumumab. In some embodiments, the EGFR inhibitor is vandetanib. In some embodiments, the EGFR inhibitor is afatinib. In some embodiments, the EGFR inhibitor is desmethyl erlotinib. In some embodiments, the EGFR inhibitor is CO-1686. In some embodiments, the EGFR inhibitor is AP26113.

In some embodiments, the pharmaceutical composition is used in treating an EGFR inhibitor resistant cancer. In some embodiments, the pharmaceutical composition is useful in treating an EGFR inhibitor resistant cancer. In some embodiments of the pharmaceutical composition, the EGFR inhibitor resistant cancer is selected from the group consisting of lung cancer, pancreatic cancer, breast cancer, colon cancer, esophageal cancer, thyroid cancer, liver cancer, glioblastoma, and astrocytoma-glioblastoma. In some embodiments of the pharmaceutical composition, the EGFR inhibitor resistant cancer is non-small cell lung cancer. In some embodiments of the pharmaceutical composition, the EGFR inhibitor resistant cancer is a metastatic cancer. In some embodiments of the pharmaceutical composition, the EGFR inhibitor resistant cancer is resistant to an EGFR inhibitor selected from the group consisting of gefitinib, erlotinib, cetuximab, lapatinib, panitumumab, vandetanib, afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626, zalutumumab, nimotuzumab, matuzumab, AP26113, and CO-1686. In some embodiments the pharmaceutical composition is useful for treating cancers that do not include an EGFR T790M mutation, do not include an increased level of MET, and/or do not include the presence of an EMT. In some embodiments the pharmaceutical composition is useful for treating cancers that include an EGFR T790M mutation, an increased level of MET, and/or the presence of an EMT.

The pharmaceutical compositions include pharmaceutically acceptable salts of the modulators disclosed herein. The compound included in the pharmaceutical composition may be covalently attached to a carrier moiety. Alternatively, the compound included in the pharmaceutical composition is not covalently linked to a carrier moiety.

The compounds of the invention (i.e. compounds described herein, including embodiments, examples) can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation).

The compounds of the present invention can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compounds of the present invention can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, that may also act as diluents, flavoring agents, binders, preservatives, tablet disintegrating agents, or an encapsulating material.

In powders, the carrier is a finely divided solid in a mixture with the finely divided active component (e.g. a compound provided herein). In tablets, the active component is mixed with the carrier having the necessary binding properties in suitable proportions and compacted in the shape and size desired.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. The compounds of the invention can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component (e.g. compounds described herein, including embodiments, examples) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Pharmaceutical compositions provided by the present invention include compositions wherein the active ingredient (e.g. compounds described herein, including embodiments, examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule and/or reducing, eliminating, or slowing the progression of disease symptoms (e.g. cancer growth or metastasis). Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. lung cancer, non-small cell lung cancer, pancreatic cancer, breast cancer, colon cancer, esophageal cancer, thyroid cancer, liver cancer, glioblastoma, astrocytoma-glioblastoma, or EGFR inhibitor resistant forms thereof), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In one embodiment, the dosage range is 0.001% to 10% w/v. In another embodiment, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compositions of the present invention can be delivered by transdermally, by a topical route, formulated as applicator sticks, solutions, suspensions, emulsions, gels, creams, ointments, pastes, jellies, paints, powders, and aerosols. For therapeutic applications, the compounds or drugs of the present invention can be administered alone or co-administered in combination with conventional chemotherapy, radiotherapy, hormonal therapy, and/or immunotherapy.

The pharmaceutical compositions of the present invention can be provided as a salt and can be formed with many acids, including but not limited to hydrochloric, sulfuric, acetic, lactic, tartaric, malic, succinic, etc. Pharmaceutical compositions described herein may be salts of a compound or composition which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder that is combined with buffer prior to use.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

Certain compositions described herein can exist in unsolvated forms as well as solvated forms, including hydrated forms. In general, the solvated forms are equivalent to unsolvated forms and are intended to be encompassed within the scope of the present invention. Certain compositions described herein may exist in multiple crystalline or amorphous forms. In general, all physical forms are equivalent for the uses contemplated by the present invention and are intended to be within the scope of the present invention.

In another embodiment, the compositions of the present invention are useful for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

Formulations suitable for oral administration can comprise: (a) liquid solutions, such as an effective amount of a packaged compound or drug suspended in diluents, e.g., water, saline, or PEG 400; (b) capsules, sachets, or tablets, each containing a predetermined amount of moduloator, compound or drug, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise a modulator, compound or drug in a flavor, e.g., sucrose, as well as pastilles comprising the modulator or compound in an inert base, such as gelatin and glycerin or sucrose and acacia emulsions, gels, and the like, containing, in addition to the modulator, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which comprises an effective amount of a compound or drug with a suppository base. Suitable suppository bases include natural or synthetic triglycerides or paraffin hydrocarbons. In addition, it is also possible to use gelatin rectal capsules which contain a combination of the compound or drug of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, orally, topically, intraperitoneally, intravesically, or intrathecally. Parenteral administration, oral administration, and intravenous administration are the preferred methods of administration. The formulations of compounds can be presented in unit-dose or multi-dose sealed containers, such as ampoules and vials.

In therapeutic use for the treatment of cancer, compound utilized in the pharmaceutical compositions of the present invention may be administered at dosages, that may be varied depending upon the requirements of the patient, the severity of the condition being treated, and the compound or drug being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The compounds described herein can be used in combination with one another, with other active agents known to be useful in treating cancer or with adjunctive agents that may not be effective alone, but may contribute to the efficacy of the active agent.

### Additional Embodiments

1. A method of detecting an increased level of AXL or GAS6 in a cancer patient, the method comprising: (i) obtaining a sample from a cancer patient; and (ii) detecting an increased level of AXL or GAS6 in said sample relative to a control.
2. The method of embodiment 1, wherein said cancer patient has a cancer comprising cancer cells with an EGFR-activating mutation.
3. The method of embodiment 1, wherein the EGFR-activating mutation is an exon-19 deletion or an exon-21 point mutant.
4. The method of any one of embodiments 1 to 3, wherein the method does not comprise detecting a level of an EMT marker except AXL.
5. The method of any one of embodiments 1 to 4, wherein said detecting comprises: (a) contacting the sample with a detectable AXL-binding agent or detectable GAS6-binding agent; (b) allowing said detectable AXL-binding agent and AXL to form an AXL complex or said GAS6-binding agent and GAS6 to form a GAS6 complex; and (c) detecting said AXL complex or GAS6 complex.
6. The method of any one of embodiments 1 to 5, wherein said detecting comprises detecting a level of an AXL or GAS6 nucleic acid or fragment thereof.
7. The method of any one of embodiments 1 to 6, wherein said detecting comprises use of nucleic acid amplification, a gene array, a microarray, a macroarray, a DNA array, or a DNA chip.
8. The method of any one of embodiments 1 to 5, wherein said detecting comprises detecting a level of an AXL or GAS6 protein or fragment thereof.
9. The method of any one of embodiments 1 to 8, wherein said detecting comprises use of an antibody, flow cytometry, ELISA, mass spectroscopy, immunofluorescence, or fluorescence microscopy.
10. The method of embodiment 9, wherein said antibody is conjugated to a detectable moiety.
11. The method of any one of embodiments 1 to 10 comprising detecting an increased level of AXL.
12. The method of any one of embodiments 1 to 11, wherein the level of AXL is a level of AXL mRNA, AXL protein, or AXL kinase activity.
13. The method of any one of embodiments 1 to 12, wherein said cancer patient is an EGFR TKI resistant cancer patient, wherein said EGFR TKI resistant cancer patient has a cancer that is not a mesenchymal cancer.
14. The method of embodiment 13, wherein said EGFR TKI resistant cancer patient is resistant to gefitinib, erlotinib, cetuximab, lapatinib, panitumumab, vandetanib, afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626, zalutumumab, nimotuzumab, matuzumab, AP26113, or CO-1686.
15. The method of any one of embodiments 13 to 14, wherein the EGFR TKI resistant cancer patient is resistant to erlotinib or gefitinib.
16. The method of any one of embodiments 1 to 15, wherein said cancer patient has a cancer selected from the group consisting of lung cancer, pancreatic cancer, breast cancer, colon cancer, esophageal cancer, thyroid cancer, liver cancer, glioblastoma, and astrocytoma-glioblastoma.
17. The method of embodiment 16, wherein said lung cancer is non-small cell lung cancer (NSCLC).
18. The method of any one of embodiments 1 to 17, wherein said cancer is metastatic cancer.
19. The method of any one of embodiments 1 to 18, wherein said cancer is non-small cell lung cancer comprising EGFR having an exon-19 deletion or an exon-21 point mutant; and further wherein the cancer is erlotinib resistant or gefitinib resistant relative to a control.
20. A method of identifying an EGFR inhibitor resistant cancer patient comprising: (i) obtaining a sample from a cancer patient; (ii) detecting an increased level of AXL or GAS6 in said sample relative to a control.
21. The method of embodiment 20, wherein said EGFR inhibitor resistant cancer patient has a cancer comprising cancer cells with an EGFR-activating mutation.
22. The method of embodiment 21, wherein the EGFR-activating mutation is an exon-19 deletion or an exon-21 point mutant.
23. The method of any one of embodiments 20 to 22, wherein the method does not comprise detecting a level of an EMT marker except AXL.
24. The method of any one of embodiments 20 to 23, wherein said EGFR inhibitor resistant cancer patient has a cancer that is not a mesenchymal cancer.
25. The method of any one of embodiments 20 to 24, wherein said detecting comprises detecting a level of an AXL or GAS6 nucleic acid or fragment thereof.
26. The method of embodiment 25, wherein said detecting comprises use of nucleic acid amplification, a gene array, a microarray, a macroarray, a DNA array, or a DNA chip.
27. The method of any one of embodiments 20 to 26, wherein said detecting comprises detecting a level of an AXL or GAS6 protein or fragment thereof.
28. The method of embodiment 27, wherein said detecting comprises use of an antibody, flow cytometry, ELISA, mass spectroscopy, immunofluorescence, or fluorescence microscopy.
29. The method of embodiment 28, wherein said antibody is conjugated to a detectable moiety.
30. The method of any one of embodiments 20 to 29, wherein said EGFR inhibitor resistant cancer patient is resistant to gefitinib, erlotinib, cetuximab, lapatinib, panitumumab, vandetanib, afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626, zalutumumab, nimotuzumab, matuzumab, AP26113, or CO-1686.
31. The method of any one of embodiments 20 to 30, wherein the EGFR inhibitor resistant cancer patient is resistant to erlotinib or gefitinib.
32. The method of any one of embodiments 20 to 31, wherein said EGFR inhibitor resistant cancer patient has a cancer selected from the group consisting of lung cancer, pancreatic cancer, breast cancer, colon cancer, esophageal cancer, thyroid cancer, liver cancer, glioblastoma, and astrocytoma-glioblastoma.
33. The method of embodiment 32, wherein said lung cancer is non-small cell lung cancer (NSCLC).
34. The method of any one of embodiments 20 to 33, wherein said cancer is metastatic cancer.
35. The method of any one of embodiments 20 to 34, wherein said cancer is non-small cell lung cancer comprising EGFR having an exon-19 deletion or an exon-21 point mutant; and further wherein the cancer is erlotinib resistant or gefitinib resistant relative to a control.
36. The method of any one of embodiments 20 to 35 comprising detecting an increased level of AXL.
37. The method of any one of embodiments 20 to 36, wherein the level of AXL is a level of AXL mRNA, AXL protein, or AXL kinase activity.

### EXAMPLES

### AXL Promotes Resistance In Vivo and In Vitro

Human NSCLCs with activating mutations in EGFR frequently respond to treatment with EGFR tyrosine kinase inhibitors (TKIs) such as erlotinib but responses are not durable as tumors acquire resistance. Secondary mutations in EGFR (T790M) or upregulation of the MET kinase are found in over 50% of resistant tumors. Described herein is increased activation of AXL and evidence of epithelial-to-mesenchymal transition (EMT) in multiple in vitro and in vivo EGFR-mutant lung cancer models with erlotinib acquired resistance in the absence of EGFR T790M or MET activation. Genetic or pharmacologic inhibition of AXL restored sensitivity to erlotinib in these tumor models. Increased expression of AXL, and in some cases its ligand GAS6, was found in EGFR-mutant lung cancers obtained from patients with EGFR TKI acquired resistance. These data identify AXL as a promising therapeutic target whose inhibition could prevent or overcome EGFR TKI acquired resistance in EGFR-mutant lung cancer patients.

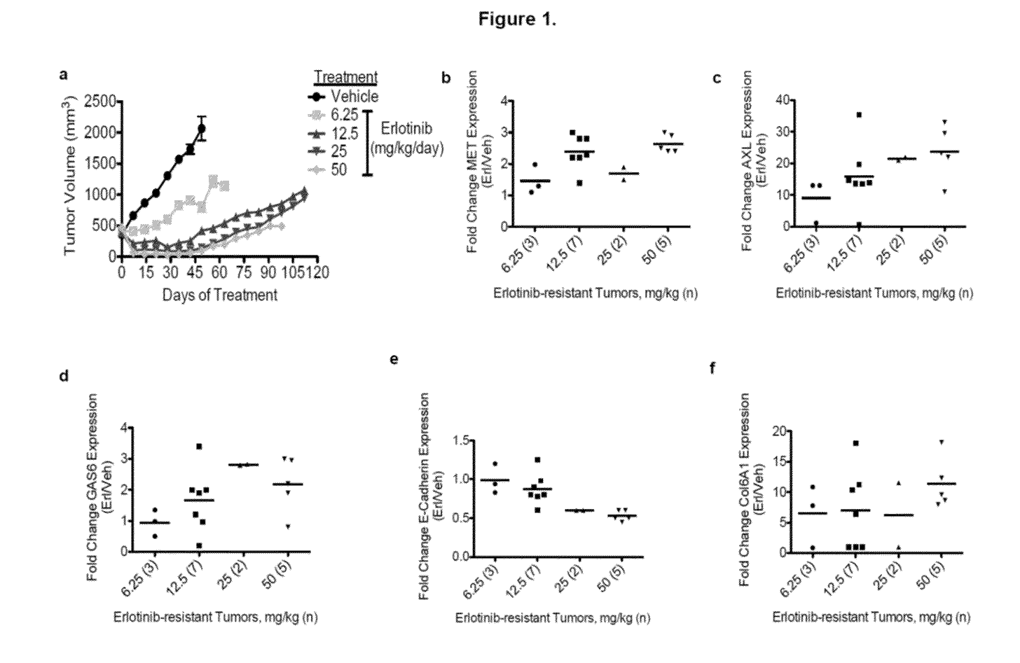

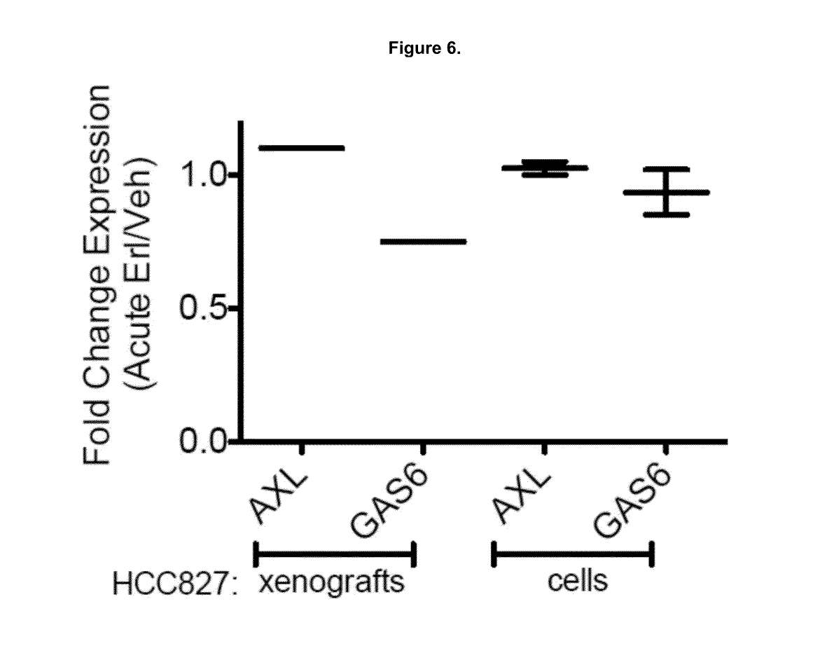

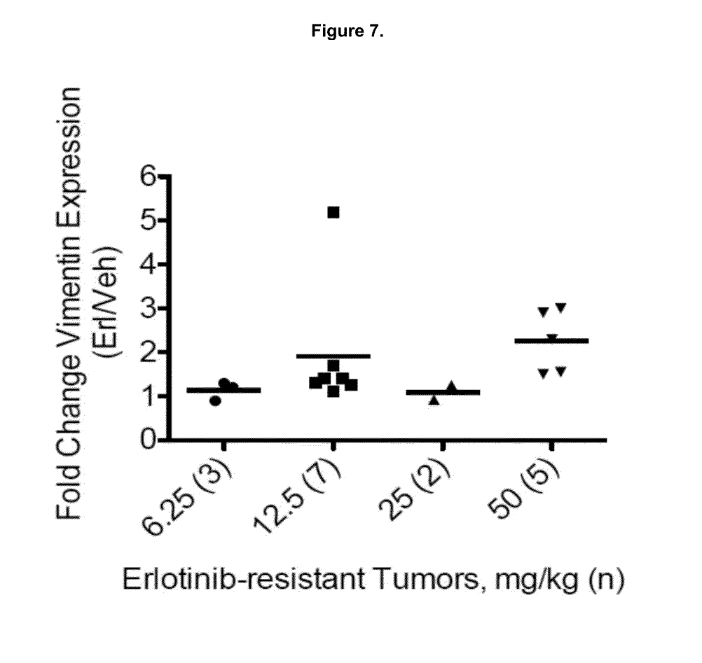

To identify novel mechanisms of acquired resistance to EGFR TKI treatment, new in vivo and in vitro (n=5) models of acquired resistance to the EGFR TKI erlotinib using EGFR exon 19 deletion mutant (delE746-A750) HCC827 human NSCLC cells were established. HCC827 cells are initially sensitive to erlotinib treatment (in vitro IC₅₀ ˜5 nM) and they have been used to develop in vitro models of EGFR TKI acquired resistance in studies that have led to the identification of clinically relevant mechanisms of EGFR TKI resistance. [Turke, A. B. et al. Cancer Cell 17, 77-88 (2010); Bivona, T. G. et al. Nature 471, 523-526 (2011)] To establish the in vivo model, cohorts of 5 mice (2 tumors/mouse) with established HCC827 tumors were treated with vehicle or 4 escalating doses of erlotinib (from 6.25 mg/kg/day to 50 mg/kg/day) over ˜5 months to derive erlotinib-resistant tumors. Erlotinib treatment of HCC827 xenograft tumors (10 tumors/dose, daily treatment) resulted in an initial dose-dependent decrease in tumor volume and the subsequent development of acquired resistance (>25% re-growth from max reduction) after 6-10 weeks of treatment in each tumor (FIG. 1a, Table 1). Sequencing of EGFR in each erlotinib resistant tumor showed that none harbored the EGFR T790M mutation nor other secondary mutations in EGFR associated with erlotinib resistance (D761Y, L474S, T854A). To examine whether the erlotinib resistant tumors harbored increased expression of either known or potential novel drivers of resistance, microarray expression profiling of 17 xenograft tumors across each treatment group as well as 2 vehicle treated control tumors were conducted. We asked which genes were differentially regulated in the erlotinib resistant tumors compared to the control tumors (unpaired T-test, P<0.05). The analysis showed that 21 genes were increased (≧1 log₂ fold change) specifically in the erlotinib resistant tumors. Unexpectedly, we found that the receptor tyrosine kinase AXL was the most highly overexpressed gene in the tumors with acquired erlotinib resistance. Consistent with prior studies [Engelman, J. A. et al. Science 316, 1039-1043 (2007); Bean, J. et al. Proc Natl Acad Sci USA 104, 20932-20937 (2007)], it was also observed that MET was among the genes upregulated, although to a much lesser extent that AXL. Increased mRNA expression of MET (≧1 log₂ fold change) in 5/17 (29%) of the tumors with erlotinib acquired resistance (FIG. 1b) was found. The analysis did not identify overexpression of IGF-1R, Ras, or EGFR in the erlotinib-resistant tumors. Compared to control tumors, expression of AXL was increased (≧1 log₂ fold change) in 15/17 (88%) of the tumors with erlotinib resistance (FIG. 1c). Moreover, increased expression (≧1 log₂ fold change) of GAS6, the ligand for AXL, in 8/17 (47%) of the tumors with erlotinib resistance (FIG. 1d) was found. AXL was overexpressed in each tumor that had increased MET or GAS6 levels. In 10 of the 15 tumors (66.6%) with AXL upregulation, MET overexpression was not observed. In each tumor in which AXL and MET were both increased AXL was overexpressed to a higher degree. AXL and GAS6 overexpression was unique to treatment resistant tumors and was not the result of acute effects of erlotinib treatment as their levels were not increased in erlotinib-sensitive tumors harvested after 48 h of erlotinib treatment (FIG. 6). aCGH analysis showed that neither AXL or GAS6 was amplified and sequencing of AXL revealed that it was not mutated in the erlotinib resistant tumors. Based on these findings and recent data demonstrating that AXL can promote cell growth in several cancer cell lines [Linger, R. M., Keating, A. K., Earp, H. S. & Graham, D. K. Expert Opin Ther Targets 14, 1073-1090 (2010); Keating, A. K. et al. Mol Cancer Ther 9, 1298-1307 (2010)], we hypothesized that AXL overexpression and activation may promote acquired resistance to erlotinib in EGFR-mutant NSCLCs.

AXL has been previously associated with EMT and recent studies suggest that therapeutic resistance may, in some cases, be associated with histological changes including EMT in NSCLCs. [Sequist, L. V. et al. Sci Transl Med 3, 75ra26 (2011); Vuoriluoto, K. et al. Oncogene 30, 1436-1448 (2011); Suda, K. et al. Journal of thoracic oncology: official publication of the International Association for the Study of Lung Cancer 6, 1152-1161 (2011)] We noted that the tumor xenografts with acquired erlotinib resistance harbored alterations in the expression level of several genes that are established biomarkers of EMT. For example, we found increased levels of COL6A1 (a type IV collagen), HMGA1 and HMGA2 and decreased levels of keratin genes (including KRT6A, KRT14, KRT5) in the tumors with acquired erlotinib resistance compared with the control tumors. We further examined whether the erlotinib resistant xenograft tumors exhibit molecular changes known to occur during EMT. Indeed, we found downregulation of E-cadherin (≧0.5 log₂ fold change) in 8/15 (53%) (FIG. 1e), upregulation of COL6A1 (≧1 log₂ fold change) in 14/15 (93%) (FIG. 1f) and increased vimentin (≧1 log₂ fold change) in 10/15 (66%) (FIG. 6) of the erlotinib-resistant tumors with AXL overexpression compared to the vehicle treated control tumors.

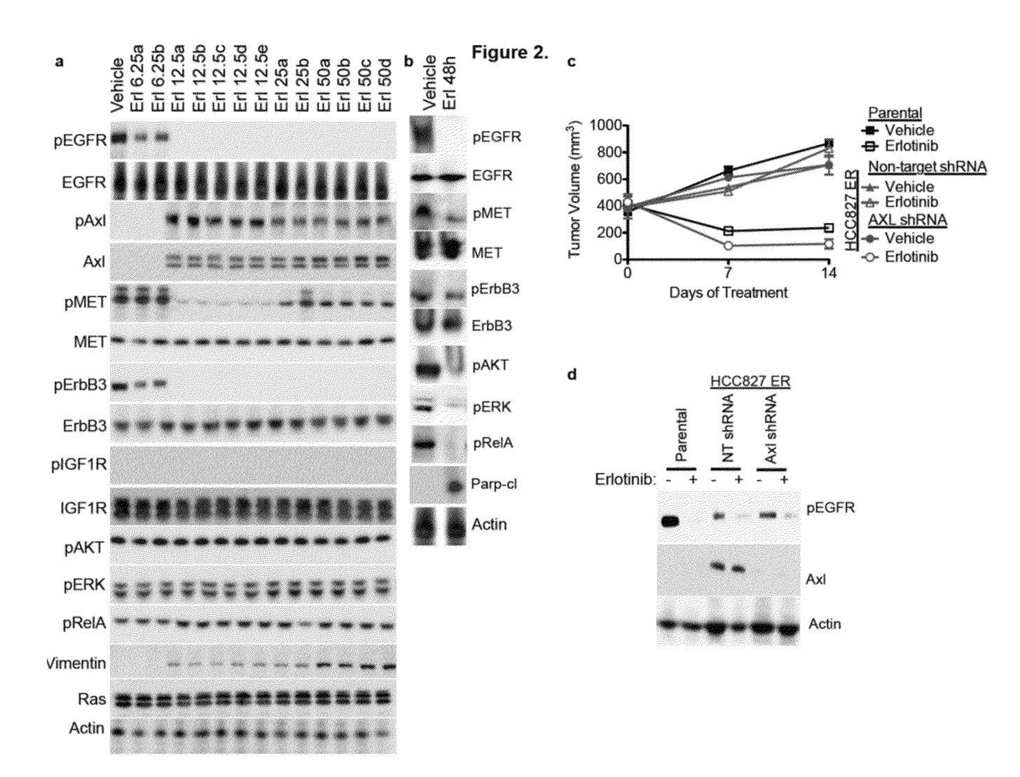

Consistent with the microarray data, we found increased levels of AXL and phosphorylated (p) AXL proteins in representative resistant HCC827 tumor xenografts at each dose of erlotinib compared with vehicle-treated tumors (FIG. 2a). We did not find alterations in the levels of IGF-1R, pIGF-1R, or Ras in the erlotinib resistant tumors (FIG. 2a). In contrast, we observed decreased levels of pEGFR, pErbB3, and pMET in response to erlotinib treatment both in sensitive tumors harvested at 48 h of treatment (FIG. 2b) and in resistant tumors at each dose of erlotinib compared to vehicle treated tumors (FIG. 2a). Erlotinib treatment for 48 h decreased pAKT, pERK, and pRelA and increased Parp cleavage in sensitive tumors (a marker of apoptosis) (FIG. 2b) but pAKT, pERK, pRelA levels were maintained in each tumor with acquired erlotinib resistance (FIG. 2a). Furthermore, we observed increased expression of the EMT marker vimentin in several erlotinib-resistant tumors (FIG. 2a). These data suggest that AXL upregulation may activate AKT, MAPK or NF-kB signaling to promote resistance to erlotinib treatment in EGFR-mutant NSCLCs perhaps in association with an EMT.

### AXL Inhibition Restores Erlotinib Sensitivity

To determine whether inhibition of AXL may restore erlotinib sensitivity in vivo, we took a genetic approach because the pharmacokinetics and specificity of many currently available AXL inhibitors in vivo are suboptimal. We generated xenograft tumors in immunocompromised mice using either parental HCC827 cells or an erlotinib resistant subclone of HCC827 cells that we established, which we found to express increased levels of AXL (HCC827 subline “ER1”, discussed further below). We transduced the AXL-overexpressing HCC827 ER1 cells with either a non-targeting shRNA or an shRNA targeting AXL prior to engraftment into the mice and then treated them with either vehicle or erlotinib (12.5 mg/kg/day). As expected, the parental HCC827 tumors were sensitive to erlotinib treatment whereas the AXL-overexpressing HCC827 ER1 tumors transduced with the non-target shRNA were erlotinib-resistant (FIG. 2c). In contrast, knockdown of AXL restored sensitivity to erlotinib in the HCC827 ER1 tumors (FIG. 2c-d). The data show that AXL was required for erlotinib resistance in this in vivo model.

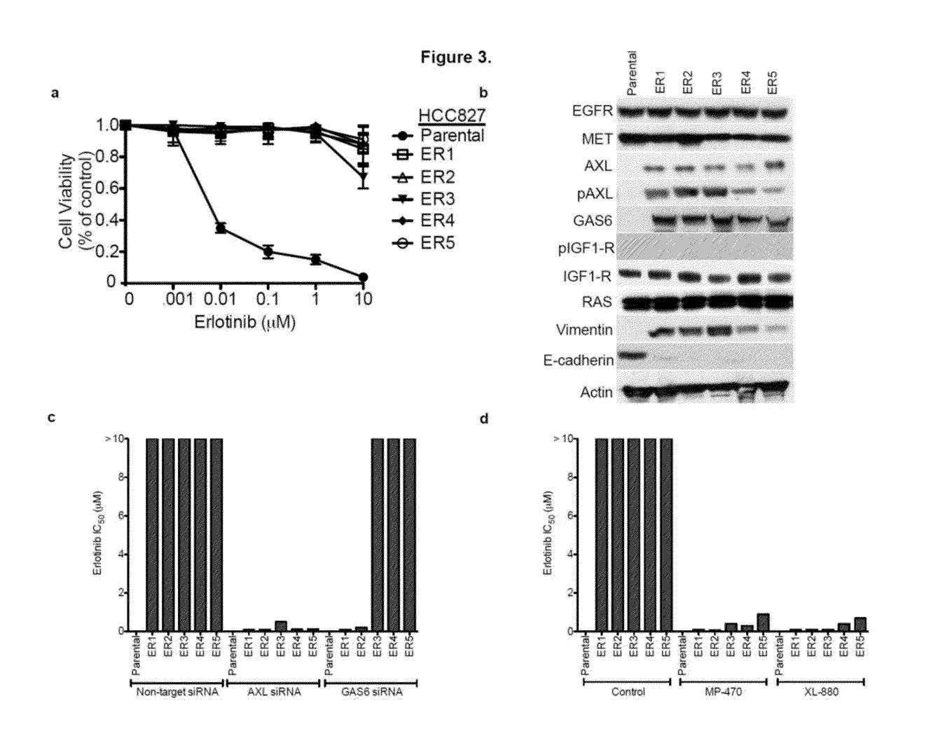

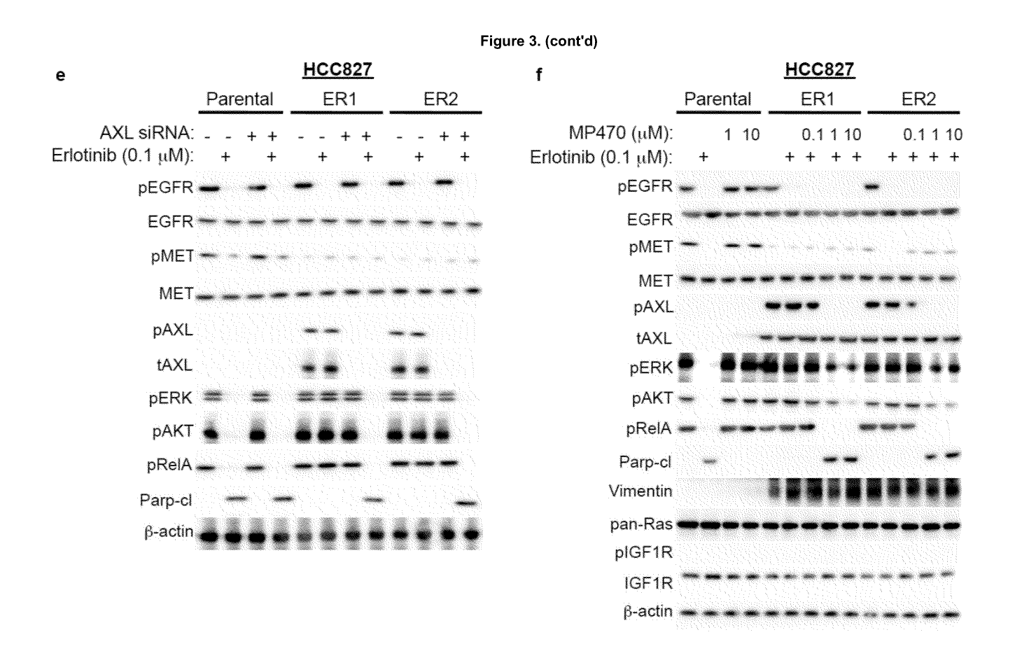

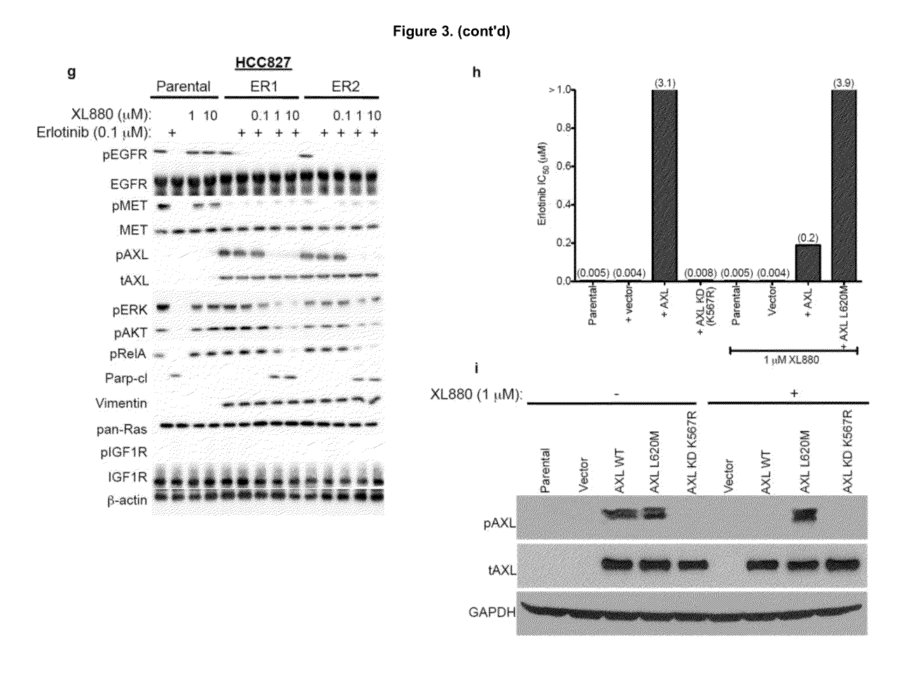

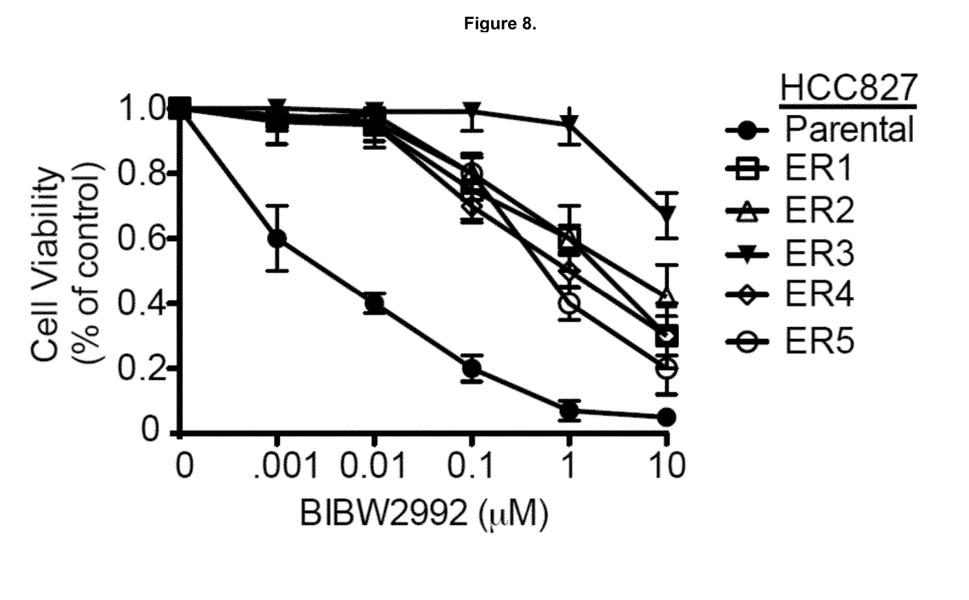

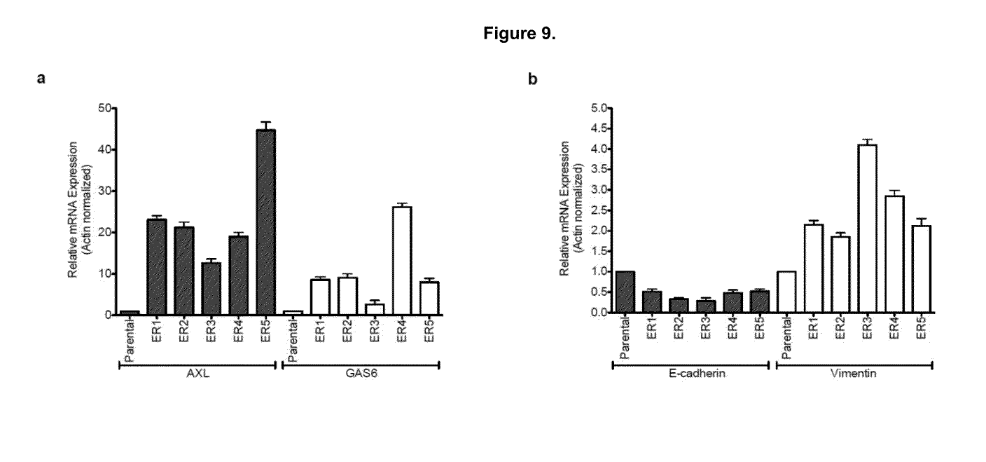

To further explore the role of AXL in EGFR TKI acquired resistance we next focused on the novel in vitro models of erlotinib acquired resistance that we generated in conjunction with the in vivo tumor xenograft models. Five erlotinib resistant HCC827 clonal sublines were established (“ER” 1-5), each with an erlotinib IC₅₀≧10 μM, through prolonged (>5 months) and continuous exposure to erlotinib (FIG. 3a). Each of these cell lines was also resistant to the irreversible EGFR kinase inhibitor BIBW2992 (afatinib) (FIG. 8). Thus resistance in these cellular models is unlikely a consequence of the secondary drug resistance mutation (T790M) in EGFR and is generalizable to distinct classes of EGFR TKIs. Indeed, the EGFR T790M resistance mutation was not detected by sequencing in any ER subline. We sought to determine whether AXL or other genes previously implicated in EGFR TKI acquired resistance were upregulated in the HCC827 ER1-5 sublines compared to parental cells. To identify genes that were differentially regulated (threshold 3-fold change, FDR<0.1) in the context of erlotinib acquired resistance, we profiled the ER1-3 sublines as compared to parental HCC827 cells by genome-wide microarrays and validated our findings by Q RT PCR and western blots in all 5 sublines. First, we noted that expression of EGFR, MET, IGF-JR, pIGF-1R and Ras was unaltered in each ER subline (FIG. 3b). We did not detect activating mutations in K-Ras or BRAF by sequencing in the ER sublines. Consistent with these findings in the erlotinib resistant tumor xenografts, we found significant upregulation of AXL and its ligand GAS6 in each HCC827 ER subline compared to HCC827 parental cells (FIG. 3b, FIG. 9). We also found increased expression of vimentin and decreased expression of E-cadherin in each ER subline (FIG. 3b, FIG. 9). Neither somatic mutations in nor genomic amplification of AXL were found by sequencing and aCGH (or FISH), respectively, in the HCC827 ER sublines.

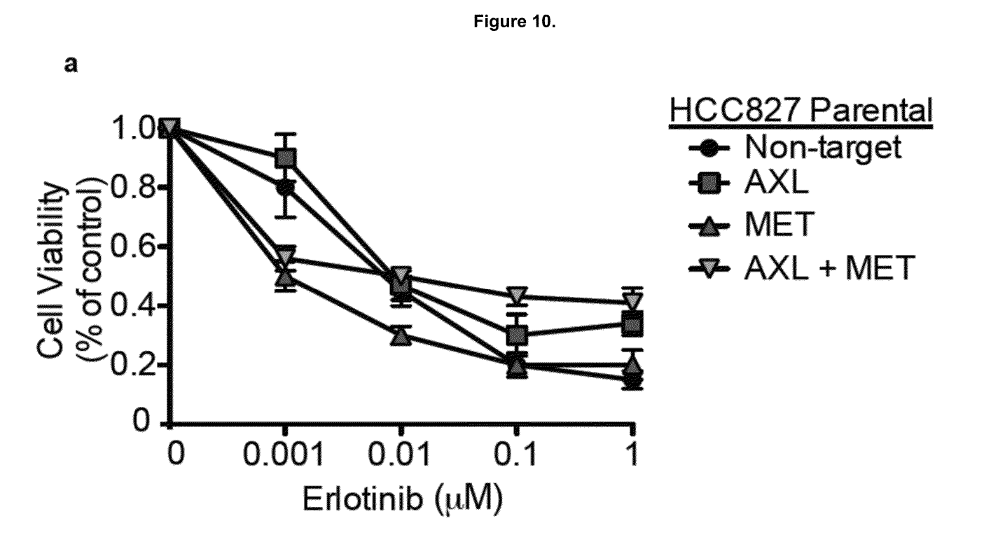

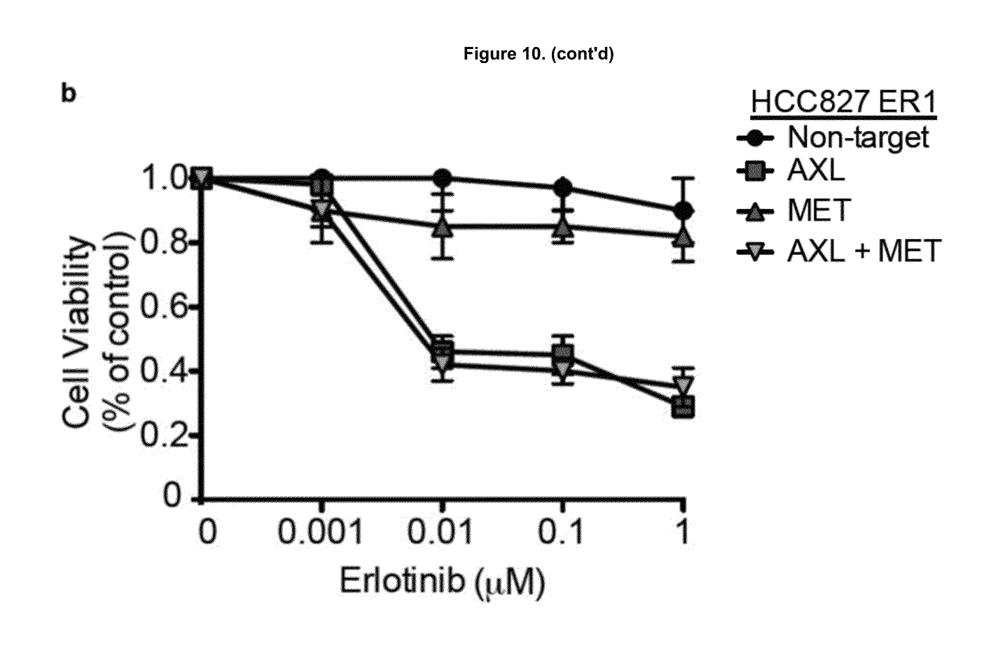

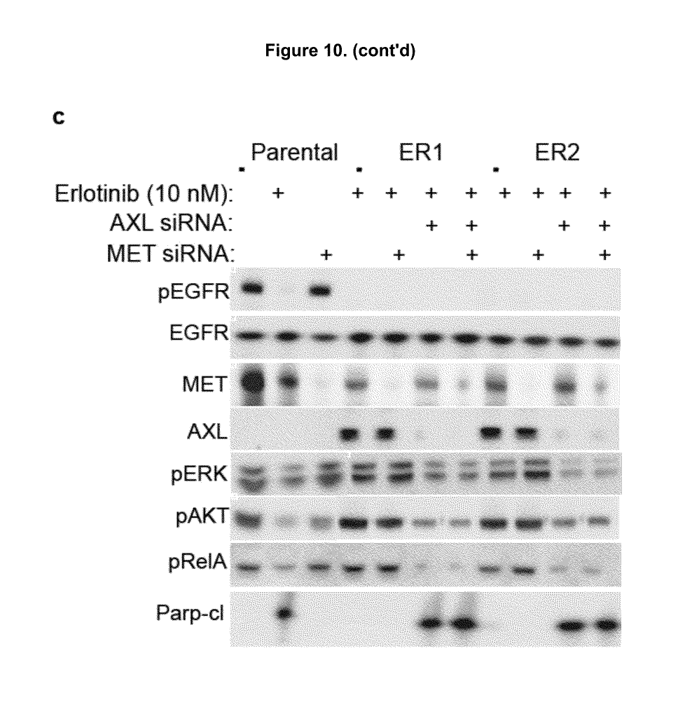

We next asked whether AXL overexpression and activation was necessary for erlotinib acquired resistance in vitro by genetically or pharmacologically inhibiting AXL in the HCC827 ER sublines. First we found that knockdown of AXL using an AXL siRNA had no effect on erlotinib sensitivity in parental HCC827 cells but restored erlotinib sensitivity in each ER subline (FIG. 3c). Interestingly, we found that knockdown of GAS6 restored erlotinib sensitivity in the ER1 and ER2 sublines but not in the ER3, ER4 or ER5 sublines (FIG. 3c). This observation is reminiscent of recent data showing that upregulation of the MET ligand HGF can promote resistance to gefitinib treatment in some EGFR mutant NSCLC cells. [Turke, A. B. et al. Cancer Cell 17, 77-88 (2010)] Because we observed residual, albeit diminished, levels of pMET in some of the ER sublines, we asked whether MET knockdown by siRNA could restore erlotinib sensitivity in this system. We found that knockdown of MET did not significantly affect erlotinib sensitivity either in parental or HCC827 ER1 cells or further enhance the effects of AXL knockdown on erlotinib sensitivity in ER1 cells (FIG. 10a, b).

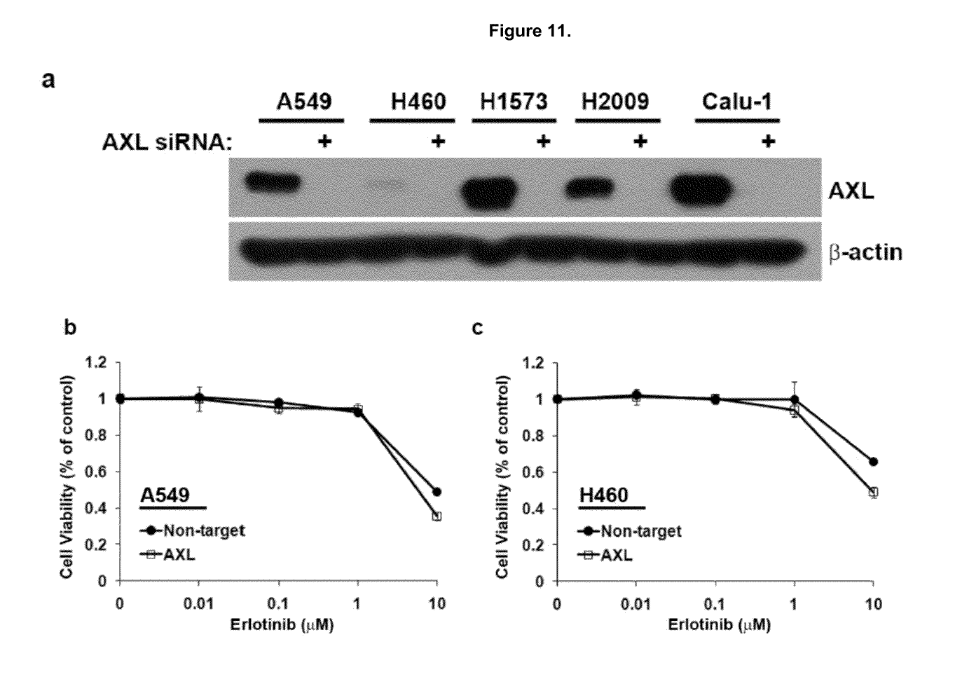

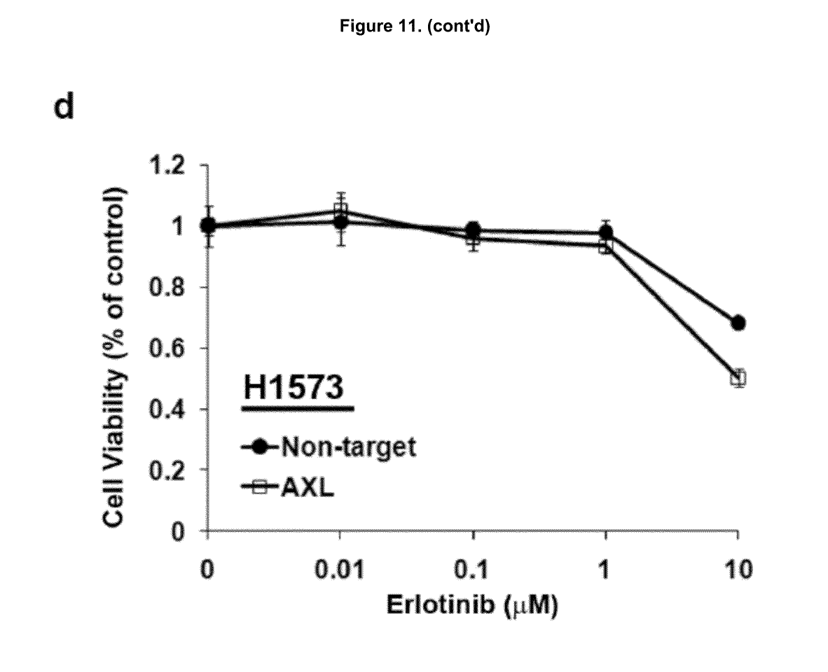

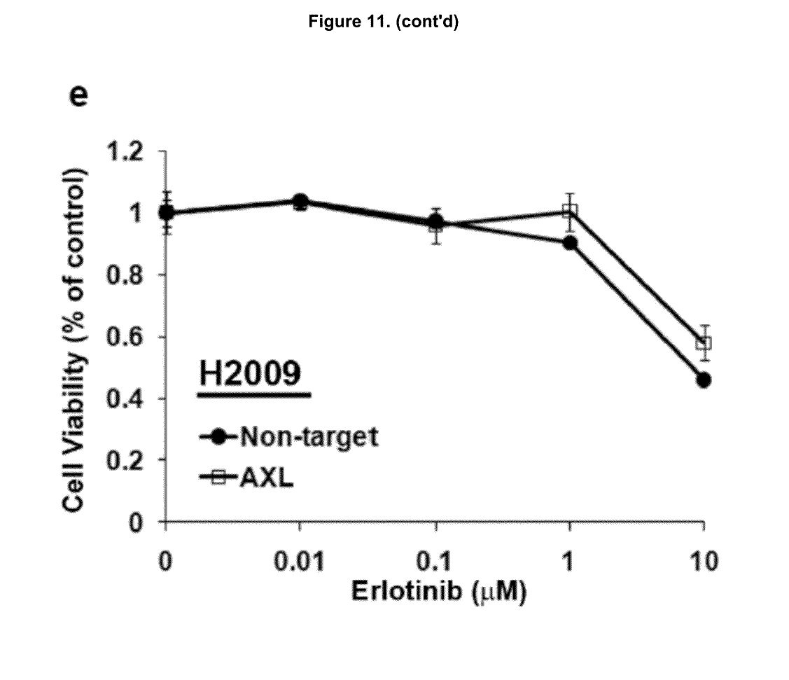

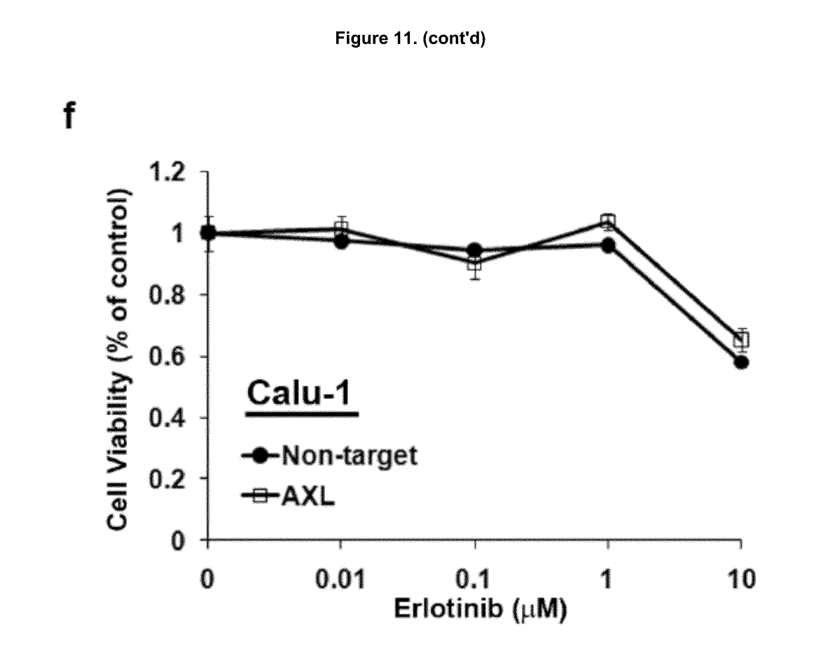

Treatment with a monoclonal antibody against AXL was recently shown to sensitize NSCLC cell lines that express wild type (WT) EGFR to erlotinib. [Ye, X. et al. Oncogene 29, 5254-5264 (2010)] Thus we asked whether knockdown of AXL by siRNA enhances erlotinib sensitivity in 5 NSCLC cell lines with WT EGFR (A549, H460, H1573, H2009, Calu-1). We found that AXL knockdown had no effect on erlotinib sensitivity in these cell lines (FIG. 11a-f). Our data suggest that inhibition of AXL enhances erlotinib sensitivity specifically in the context of NSCLCs with EGFR kinase domain activating mutations in cells with acquired EGFR TKI resistance.

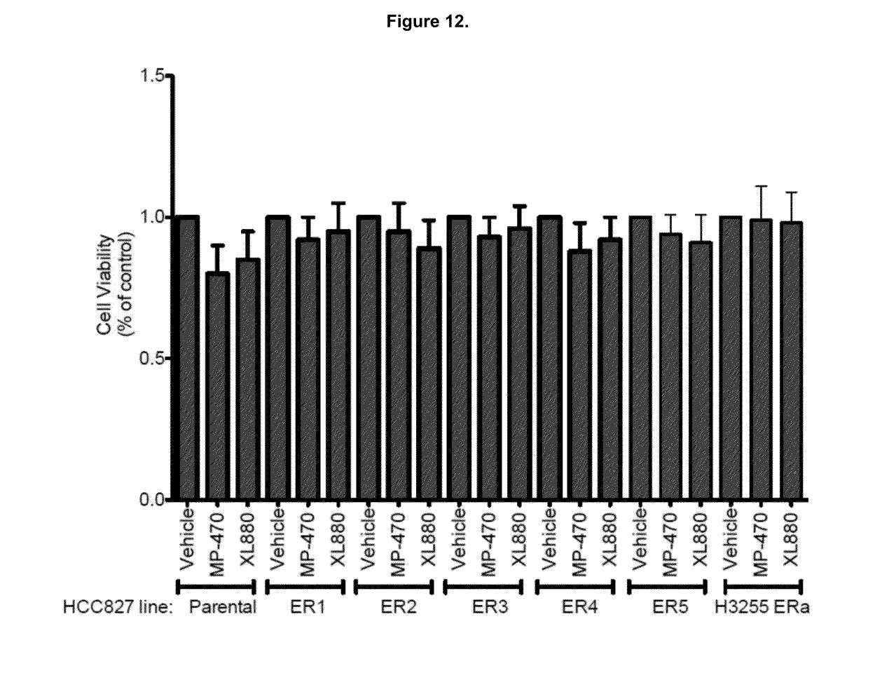

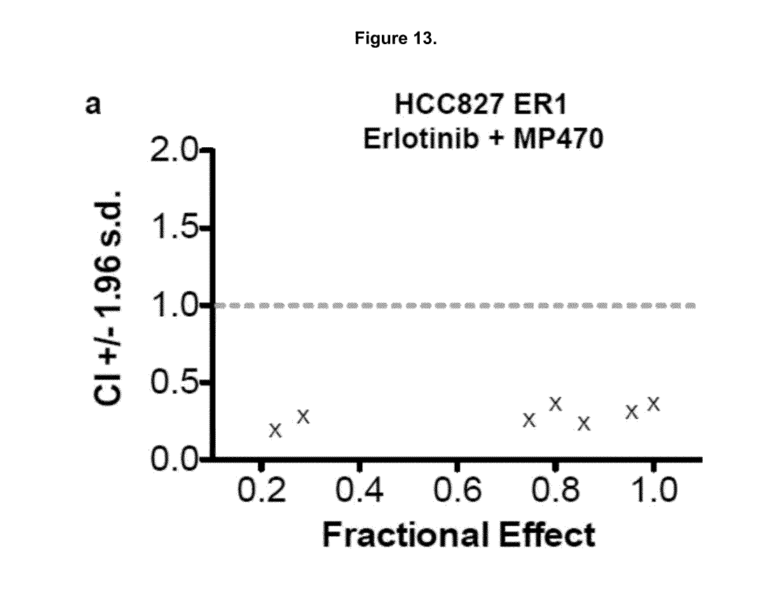

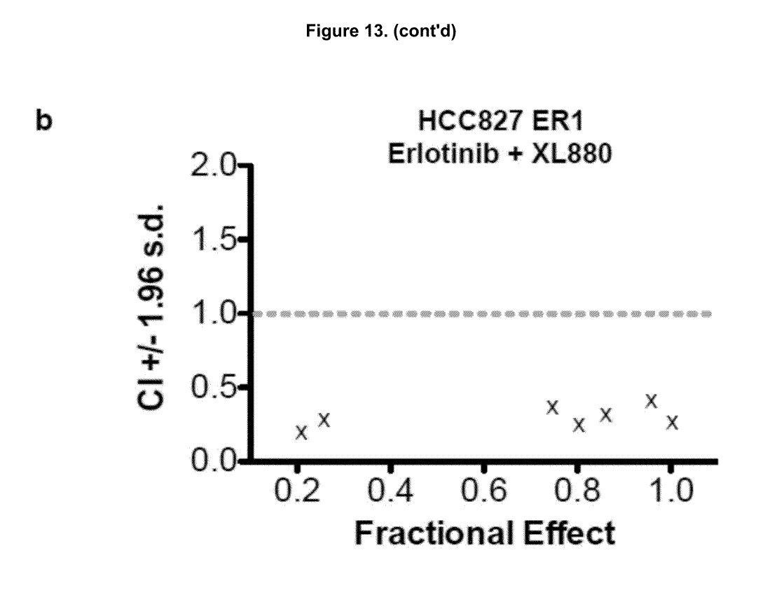

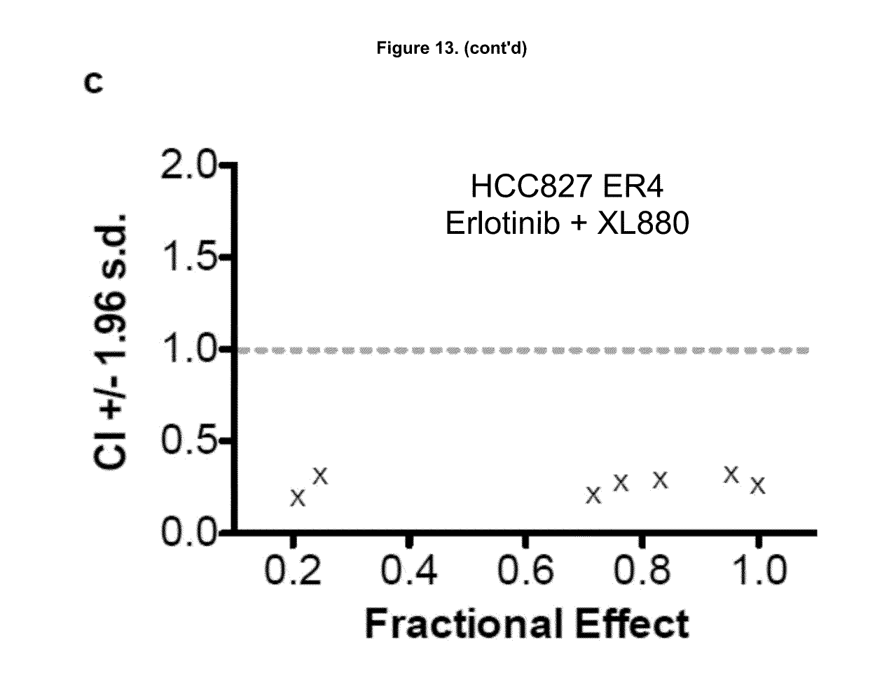

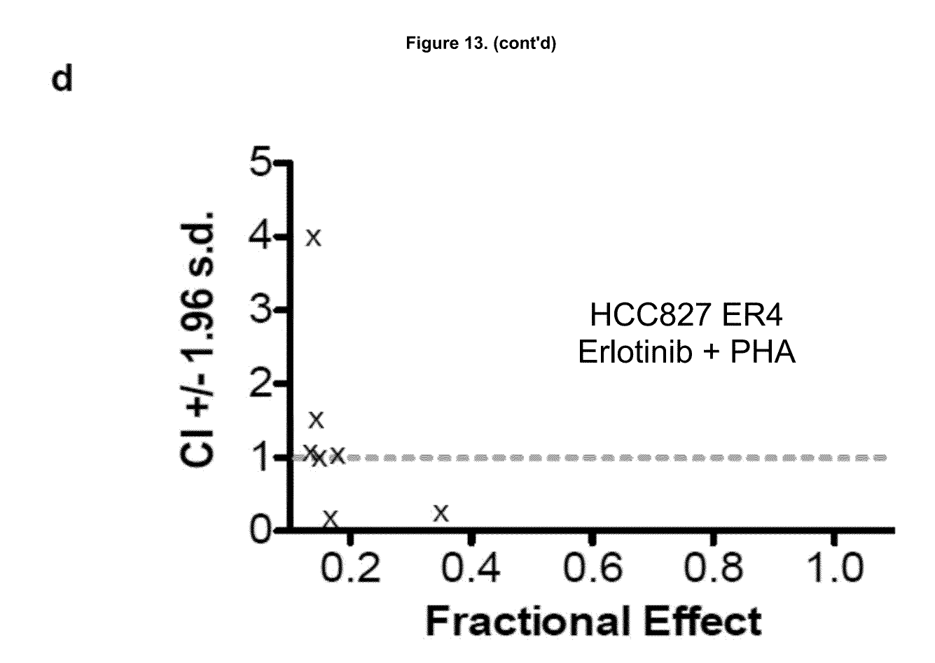

Next we sought to validate our genetic findings using 2 commercially available small molecule inhibitors of AXL, MP-470 and XL-880. [Linger, R. M., Keating, A. K., Earp, H. S. & Graham, D. K. Expert Opin Ther Targets 14, 1073-1090 (2010)] As expected, treatment with MP-470 (1 μM) or XL-880 (1 μM) alone did not significantly affect the viability of HCC827 parental or ER cells (FIG. 12). In contrast, we found that treatment with MP-470 (1 μM) or XL-880 (1 μM), restored sensitivity to concurrent erlotinib treatment in each ER subline (FIG. 3d). To determine whether treatment with an AXL inhibitor is synergistic with concurrent erlotinib treatment we conducted combination index (CI) analysis in 2 of the ER sublines (ER1 and ER4). We found that treatment with either MP470 or XL880, but not the MET inhibitor PHA665752, was synergistic with concurrent erlotinib treatment (FIG. 13a-d). Together, the genetic and pharmacologic data show that AXL is required for acquired erlotinib resistance in this system.

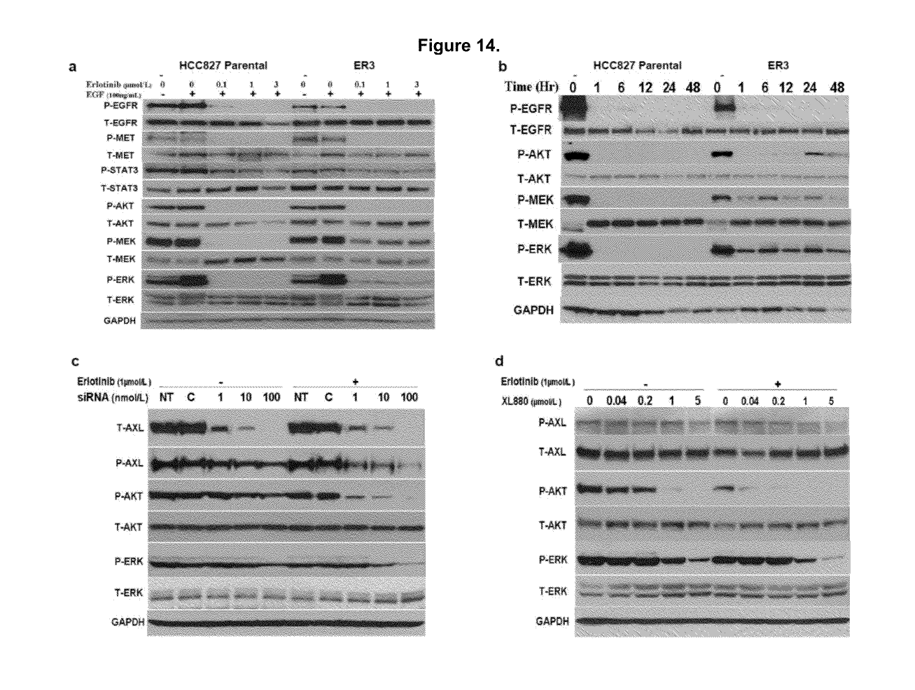

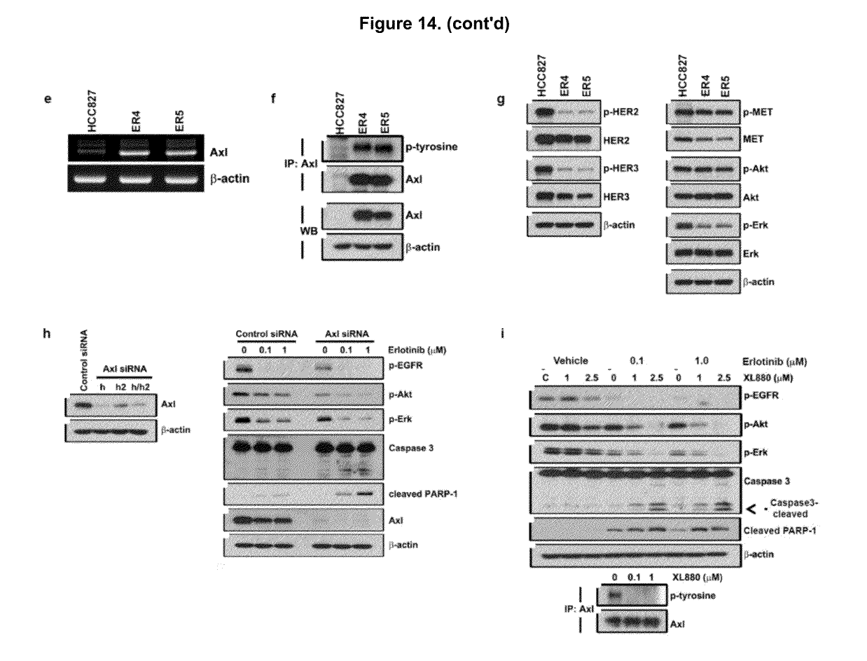

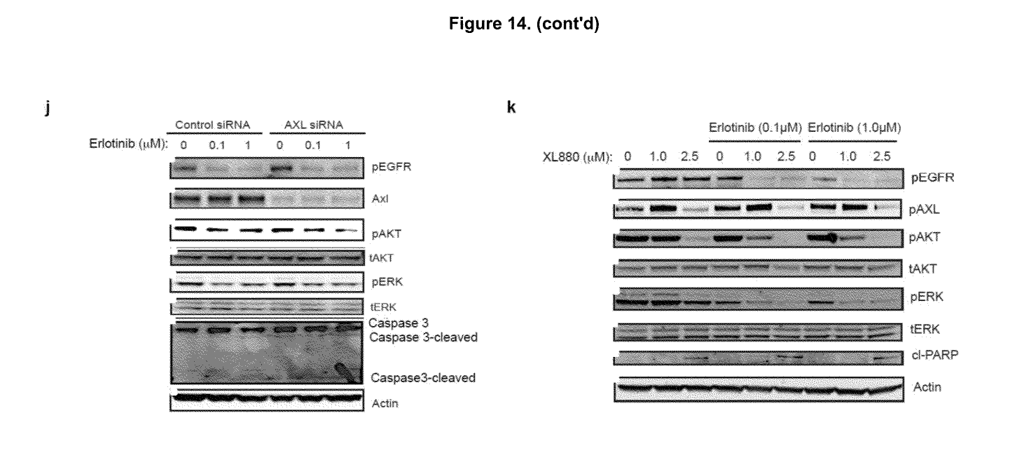

We aimed to determine which signaling events downstream of AXL might promote acquired erlotinib resistance in EGFR mutant NSCLCs. Because AXL has been shown to activate the MAPK, AKT and NF-kB pathways and regulate apoptosis in some cancer cell lines [Linger, R. M., Keating, A. K., Earp, H. S. & Graham, D. K. Expert Opin Ther Targets 14, 1073-1090 (2010); Keating, A. K. et al. Mol Cancer Ther 9, 1298-1307 (2010)], we examined activation of these pathways in HCC827 parental or ER cell lines treated with a non-targeting or AXL siRNA and either vehicle or erlotinib (100 nM). As expected, we found that erlotinib treatment decreased pERK, pAKT and pRelA (a marker of NF-kB signaling) and increased Parp cleavage (a marker of apoptosis) in parental HCC827 cells (FIG. 3e). AXL siRNA treatment had no effect on these pathways in parental HCC827 cells (FIG. 3e). Erlotinib treatment decreased pEGFR both in parental HCC827 cells and in the ER1 and ER2 sublines (FIG. 3e). This observation is consistent with our finding that the ER sublines do not harbor a secondary resistance mutation in EGFR that would abrogate the ability of erlotinib to inhibit EGFR. In contrast, erlotinib treatment decreased pERK, pAKT and pRelA and increased the levels of cleaved Parp only upon AXL knockdown in the ER1 and ER2 cell lines (FIG. 3e). Similar results were observed in HCC827 ER3, ER4 and ER5 sublines (FIG. 14a-k).

We next sought to validate our genetic findings using pharmacologic inhibitors of AXL. As expected, erlotinib decreased pEGFR, pERK, pAKT, pRelA and increased the levels of cleaved Parp in parental HCC827 cells irrespective of concurrent treatment with MP-470 or XL-880 (FIG. 3f-g). In contrast, these effects of erlotinib treatment were observed only upon concurrent treatment with MP-470 (FIG. 3f) or XL-880 (FIG. 3g) in the HCC827 ER1 and ER2 cells. Similar results were observed in HCC827 ER3, ER4 and ER5 sublines treated with erlotinib and XL-880 either alone or in combination (FIG. 14a-k). Together, the data suggest that AXL activation is necessary for acquired erlotinib resistance and that the MAPK, AKT and NF-kB pathways may, in part, mediate erlotinib resistance downstream of AXL in this system.

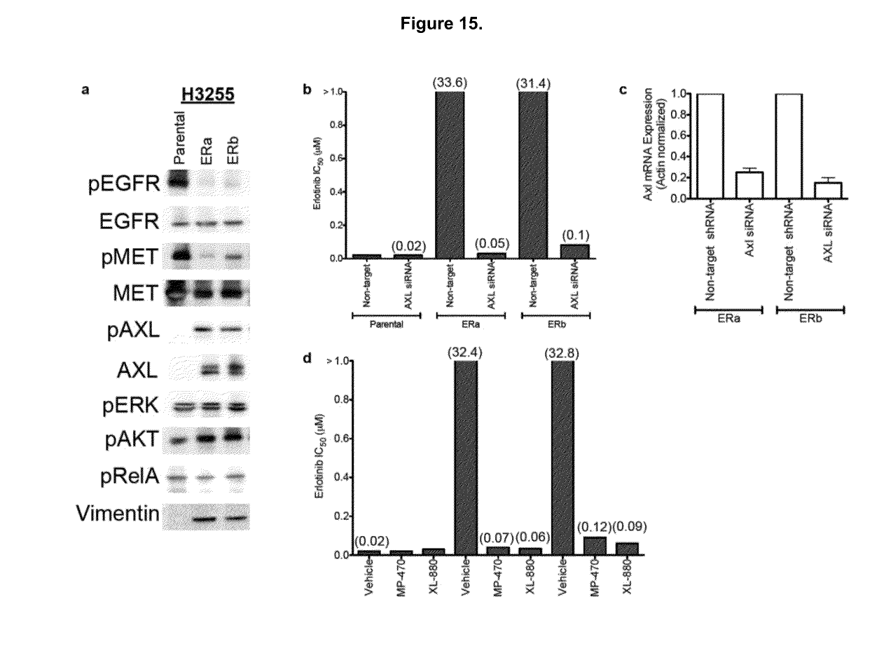

To determine whether AXL is upregulated in the setting of erlotinib acquired resistance in cells other than HCC827, we used the same erlotinib treatment protocol to establish 2 isogenic, erlotinib-resistant sublines (IC₅₀>1 μM) derived from H3255 cells that express the EGFR L858R mutant commonly found in lung cancer patients and that are otherwise sensitive to erlotinib (IC₅₀ ˜15 nM). Similar to HCC827 cells, we found (1) overexpression of AXL in the absence of EGFR T790M, pEGFR and increased pMET (2) overexpression of the EMT marker vimentin, and (3) that inhibition of AXL by siRNA, MP-470 or XL880 restored sensitivity to concurrent erlotinib treatment in the H3255 ER sublines (FIG. 15a-d). Together, these data show that AXL upregulation promotes erlotinib acquired resistance in the setting of EMT in EGFR-mutant NSCLC tumor models and that combined inhibition of AXL and EGFR overcomes acquired resistance to erlotinib in these models.

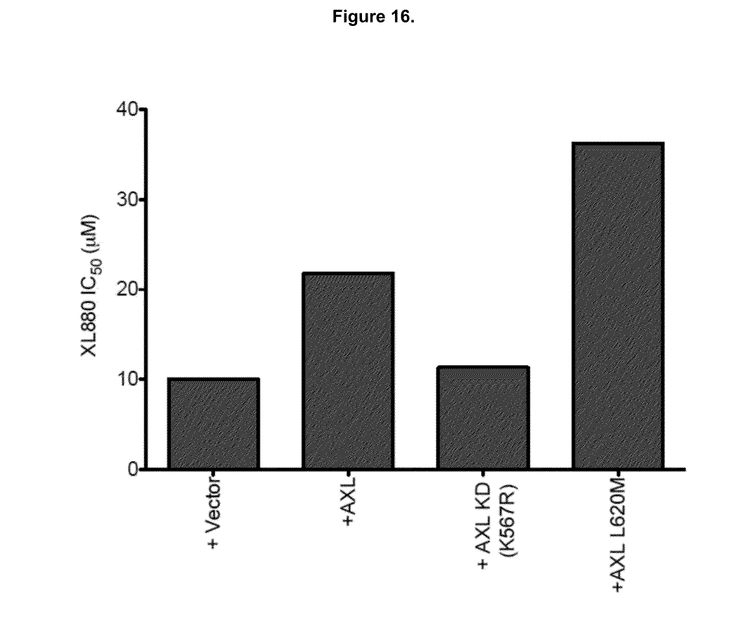

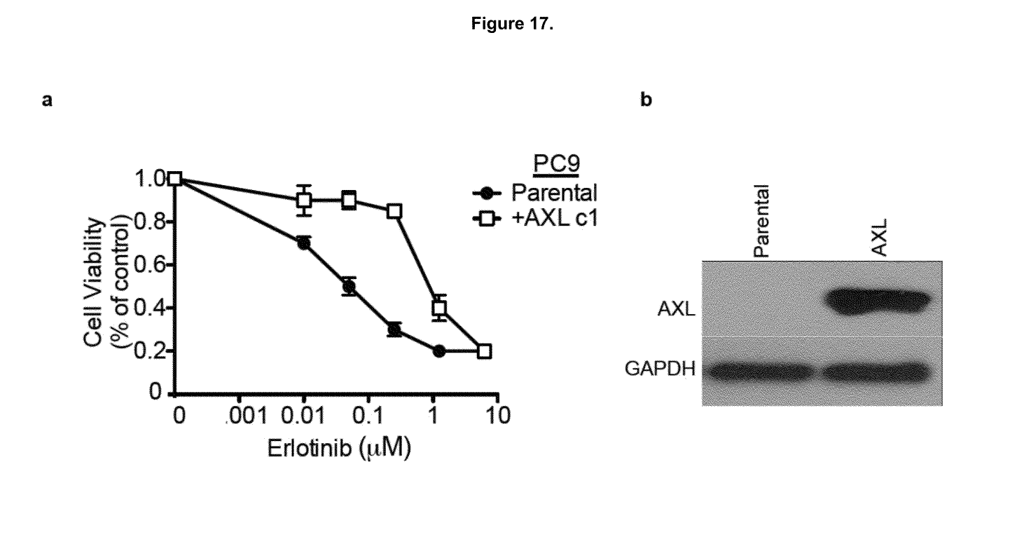

We next asked whether (1) forced expression of AXL is sufficient to induce erlotinib resistance and (2) whether AXL kinase activity is necessary for the induction of erlotinib resistance by AXL. To address these questions, we generated cDNA constructs that encode either wild type (WT) AXL or a kinase-impaired mutant of AXL in which a key conserved lysine within the kinase domain was changed to arginine (K567R). Transient overexpression of WT AXL, but not AXL KD (K567R), in HCC827 cells increased the levels of pAXL and the IC₅₀ for XL880 (FIG. 16, FIG. 3h-i), suggesting that the K567R impairs AXL kinase function. Overexpression of WT AXL, but not kinase impaired AXL KD (K567R), induced resistance to erlotinib in HCC827 cells that was reversed upon treatment with XL-880 (1 μM) (FIG. 3h-i). In addition, we found that introduction of WT AXL was sufficient to induce partial resistance to erlotinib in PC9 cells that express an EGFR exon 19-deletion mutant and are otherwise erlotinib-sensitive (IC₅₀ ˜25 nM) (FIG. 17a, b). Together, the data indicate that AXL kinase activation is necessary and sufficient to promote erlotinib resistance in these EGFR mutant NSCLC models.

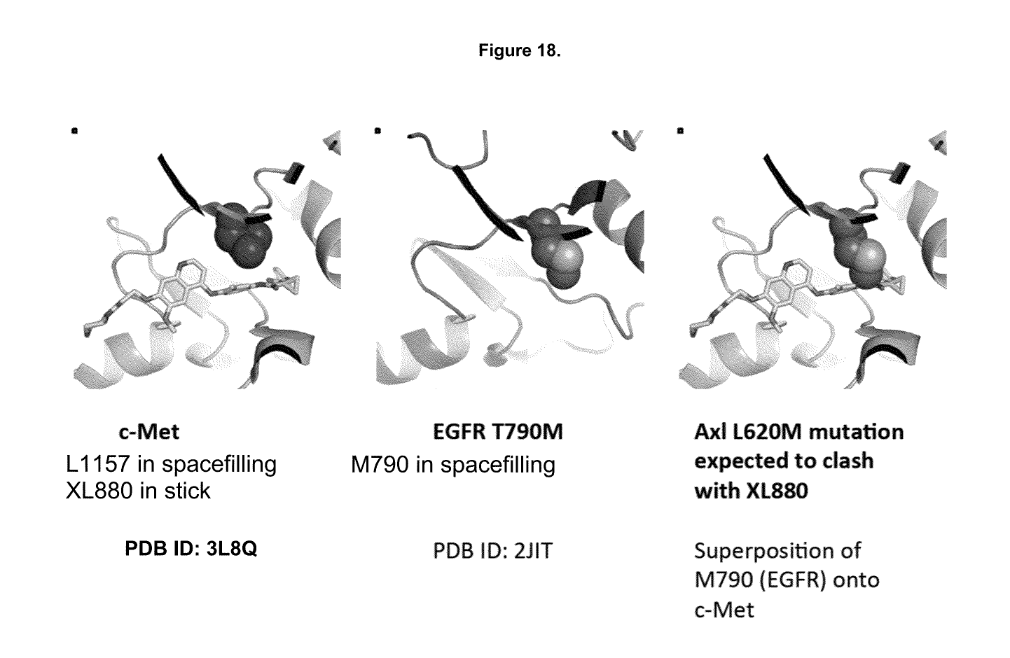

To confirm that treatment with an AXL inhibitor restores erlotinib sensitivity via inhibition of AXL, and is not a consequence of an “off-target” effect of drug treatment, we identified the “gatekeeper” residue within the AXL kinase domain based by structural modeling of the co-crystal of MET bound to XL880 and of the location of the T790M gatekeeper residue in EGFR [Qian, F. et al. Inhibition of tumor cell growth, invasion, and METastasis by EXEL-2880 (XL880, GSK1363089), a novel inhibitor of HGF and VEGF receptor tyrosine kinases. Cancer research 69, 8009-8016 (2009); Yun, C. H. et al. PNAS 105, 2070-2075 (2008)] (FIG. 18). The analysis indicated that an L620M mutation in AXL would be expected to result in steric clash with XL-880 and would block the ability of XL-880 to inhibit AXL. Thus we generated a cDNA encoding this gatekeeper mutant of AXL (L620M) and introduced it into HCC827 cells to determine if expression of it blocks the ability of XL-880 to restore erlotinib sensitivity in the setting of AXL overexpression. Expression of AXL L620M significantly increased the IC₅₀ for XL-880 (FIG. 16) and blocked the ability of XL880 to decrease pAXL in HCC827 cells (FIG. 3i). In contrast to overexpression of WT AXL, we found that expression of AXL L620M abrogated the ability of XL-880 (1 μM) to restore erlotinib sensitivity in HCC827 cells (FIG. 3h-i). The data indicate that treatment with the AXL inhibitor XL880 restores erlotinib sensitivity by inhibiting AXL kinase activation in these EGFR mutant NSCLC models.

### AXL-Mediated Resistance May Occur with an EMT

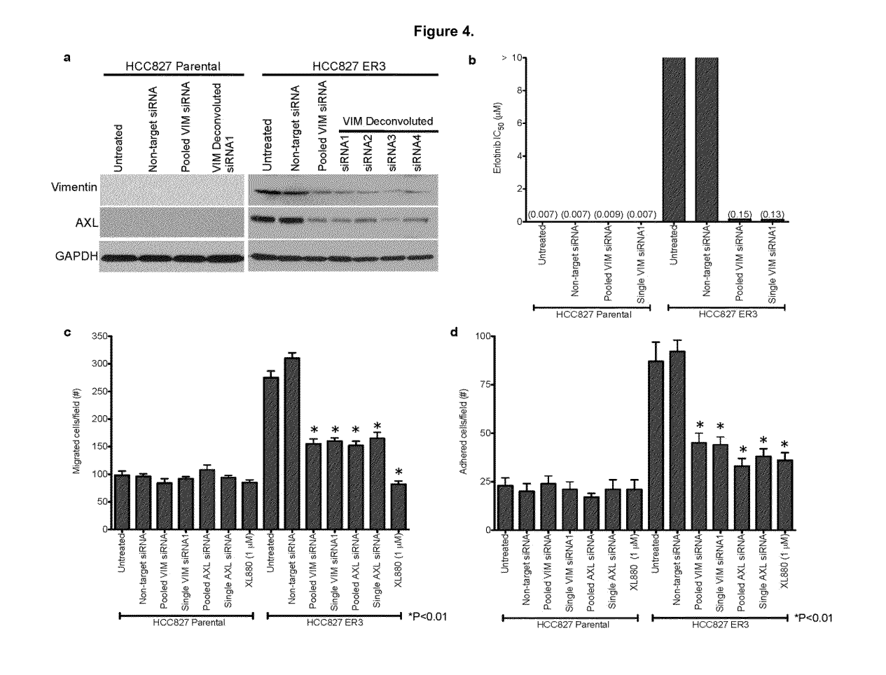

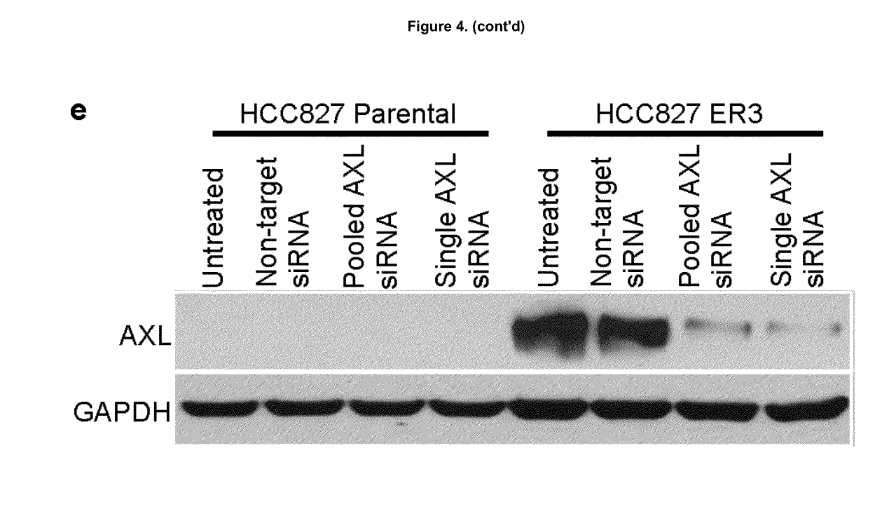

We observed an association between AXL overexpression and markers of EMT, including increased expression of vimentin, in several models of acquired erlotinib resistance. Recent data indicate that vimentin can promote EMT via epigenetic regulation of genes that are critical for EMT in breast cancer cells. [Vuoriluoto, K. et al. Oncogene 30, 1436-1448 (2011)] Thus we sought to determine whether vimentin overexpression was necessary for AXL overexpression and erlotinib resistance in HCC827 cells. We found that knockdown of vimentin by both pooled and 4 individual siRNAs decreased, but did not completely abolish, AXL expression (FIG. 4a). Vimentin knockdown and concomitant downregulation of AXL enhanced erlotinib sensitivity in HCC827 ER3 cells (FIG. 4b). Because vimentin has been shown to promote migration and adhesion in cells that have undergone an EMT [Vuoriluoto, K. et al. Oncogene 30, 1436-1448 (2011); Polyak, K. & Weinberg, R. A. Nature reviews. Cancer 9, 265-273 (2009)], we examined whether HCC827 ER cells exhibited increased migration and adhesion. We found that HCC827 cells with acquired erlotinib resistance (ER3) exhibit increased migration and adhesion compared to parental HCC827 cells (FIG. 4c-d). We also found that knockdown of vimentin, AXL, or treatment with XL880 inhibited migration and adhesion in HCC827 ER3 cells but had no effect in parental HCC827 cells (FIG. 4c-d). Together, the data support a possible role for an EMT that is marked by vimentin overexpression in the development of acquired EGFR TKI resistance that is driven by AXL in this model of human EGFR mutant lung cancer.

### AXL is Upregulated in Patients with Acquired Resistance

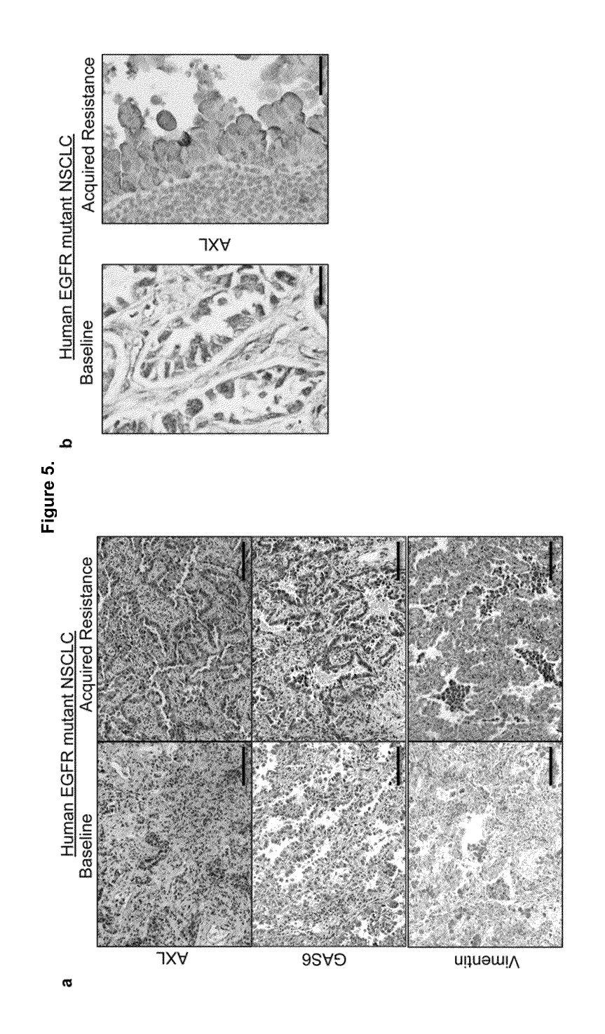

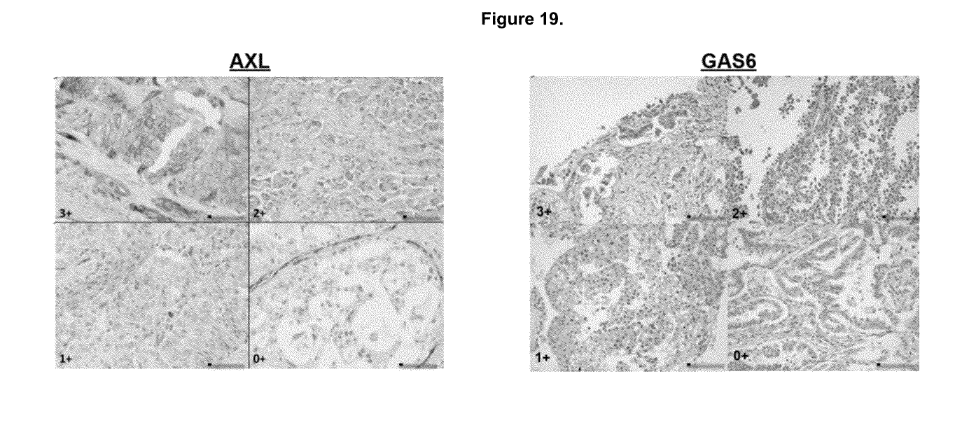

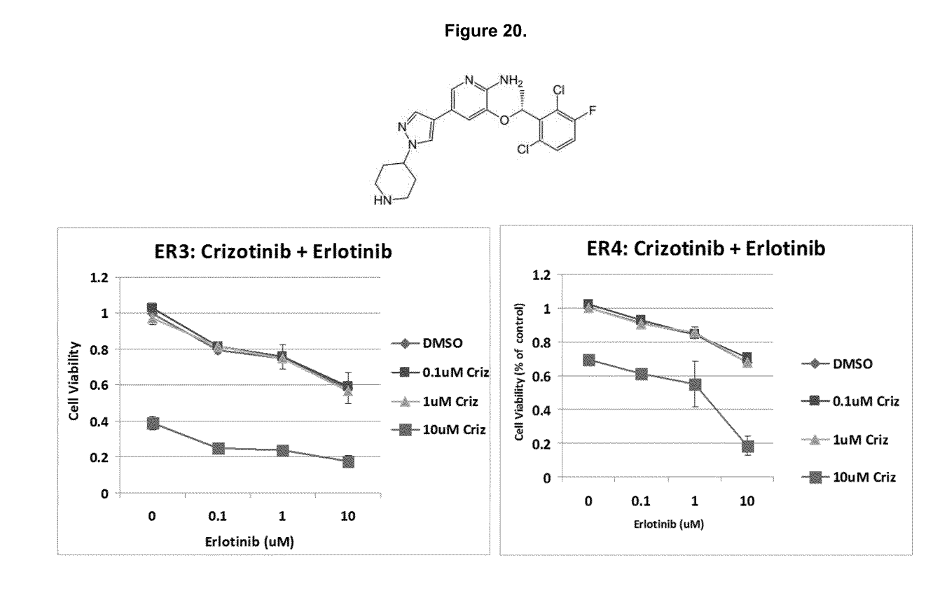

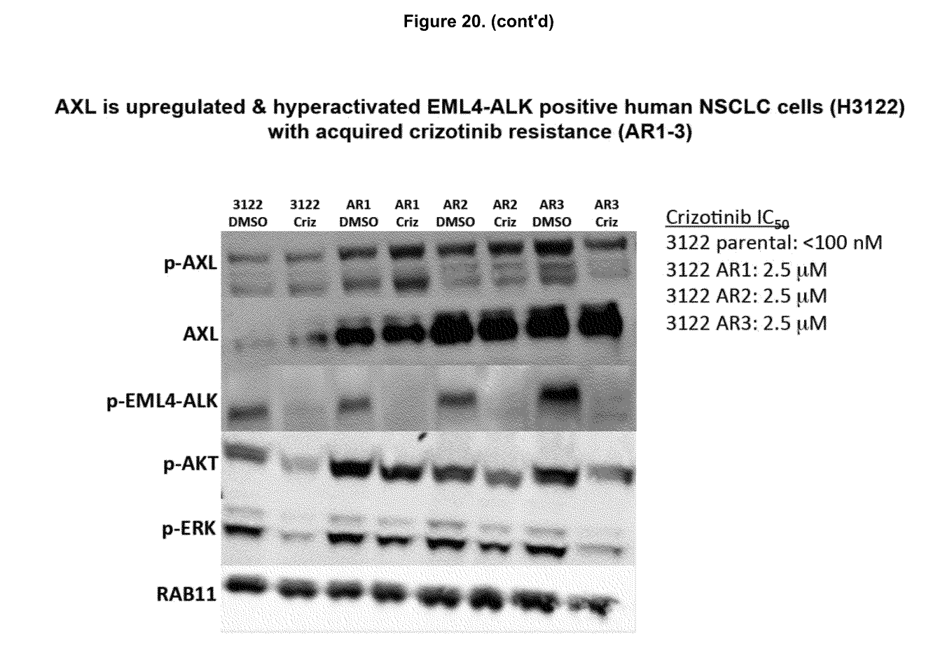

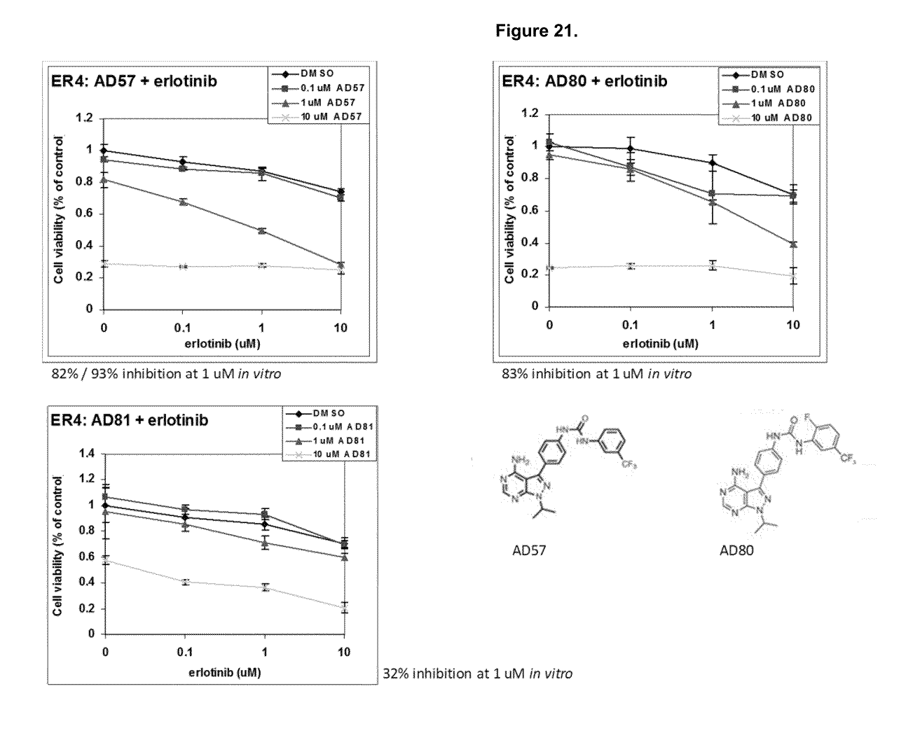

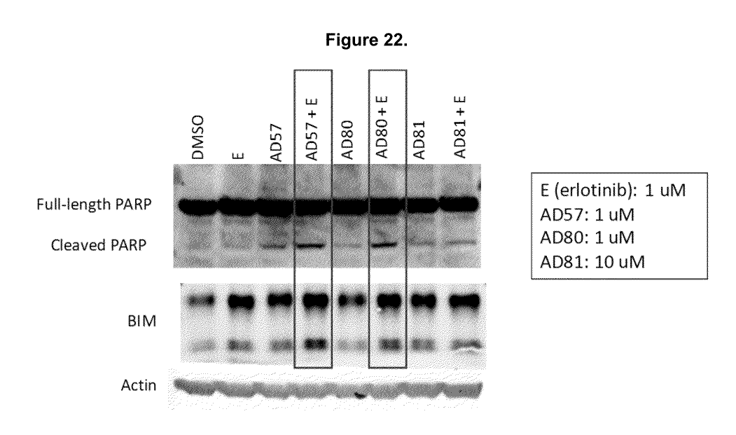

Based on the preclinical data, we hypothesized that upregulation of AXL may promote acquired resistance to EGFR TKI treatment in EGFR-mutant NSCLC patients. To test this hypothesis and to clinically validate our preclinical findings, we measured the expression of AXL by IHC (immunohistochemistry) in 35 matched EGFR-mutant NSCLC specimens obtained from patients both prior to treatment with the EGFR TKIs erlotinib or gefitinib and upon the development of EGFR TKI acquired resistance. In cases where enough material was available for additional studies, we also examined the specimens for GAS6 and vimentin (as a marker for EMT) expression by IHC (scoring system shown in FIG. 19), EGFR T790M by sequencing, and MET amplification by FISH. All patients in the cohort we analyzed had either an exon 19 deletion mutation or an exon 21 point mutation (L858R) in EGFR, were treated with either erlotinib or gefitinib and MET the established clinical definition of EGFR TKI acquired resistance (Table 2). [Jackman, D. et al. Journal of clinical oncology: official journal of the American Society of Clinical Oncology 28, 357-360 (2010)] The clinical sample set that we analyzed harbored the full spectrum of major alterations known to occur in EGFR mutant lung cancer patients with acquired EGFR TKI resistance. [Engelman, J. A. et al. Science 316, 1039-1043 (2007); Arcila, M. E. et al. Clinical cancer research: an official journal of the American Association for Cancer Research 17, 1169-1180 (2011); Bean, J. et al., Proc Natl Acad Sci USA 104, 20932-20937 (2007); Sequist, L. V. et al. Sci Transl Med 3, 75ra26 (2011)] Indeed, we detected the EGFR T790M mutation in 8/28 (29%) and MET amplification in 6/31 (19%) of the resistant specimens examined (Tables 2-3 and FIG. 5a, b). As compared to the pre-treatment specimen, we detected increased expression (an increase by 2+ or greater) of AXL in 7/35 (20%) and GAS6 in 7/28 (25%), and vimentin in 2/10 (20%) of the EGFR TKI resistant specimens evaluated (Tables 2-3 and FIG. 5a-b). In 3 cases (#13, 14, 15) in which AXL was expressed at the same level in the baseline and matched resistance specimen we detected increased GAS6 specifically in the resistant specimen (Tables 2-3). This finding is consistent with the hypothesis that GAS6 overexpression may promote AXL activation in the setting of EGFR TKI acquired resistance in some cases. This hypothesis is supported by our preclinical data showing that knockdown of GAS6 can restore erlotinib sensitivity in some ER sublines that also overexpress AXL (FIG. 3c). In order to provide independent validation of our IHC findings of increased expression of AXL and GAS6, we performed Q-RT-PCR on a small cohort of EGFR T790M mutation negative EGFR mutant lung cancer specimens with acquired EGFR TKI resistance compared to matched baseline specimens (n=5). Using stringent cut-offs (threshold >3 fold change in mRNA levels by Q RT PCR), we found increased AXL and GAS6 mRNA levels upon resistance in 20% and 60%, respectively, of these specimens. We did not find increased AXL or GAS6 in the single specimen in which we detected increased MET expression (>3 fold change in mRNA by Q RT PCR) in this small cohort.

Our analysis of the clinical specimens indicates that some EGFR mutant NSCLCs with EGFR TKI acquired resistance can harbor multiple mechanisms of resistance. Consistent with prior studies [Bean, J. et al. Proc Natl Acad Sci USA 104, 20932-20937 (2007)], we detected the EGFR T790M mutation in 50% of cases that also harbored MET amplification (Tables 2-3). Furthermore, we found co-occurrence of EGFR T790M and increased AXL and GAS6 in a minority of cases (Tables 2-3). In contrast, we did not detect MET amplification in any resistance specimen that had increased AXL or GAS6 expression (Tables 2-3). Coupled with the preclinical data, our findings suggest that activation of AXL and MET may be mutually exclusive mechanisms of acquired EGFR TKI resistance in NSCLCs.

### Data Analysis

We have identified activation of the AXL kinase as a novel mechanism of acquired EGFR TKI resistance in EGFR-mutant NSCLCs through an integrated analysis of human EGFR-mutant NSCLC tumor models and one of the largest clinical cohorts of paired EGFR-mutant NSCLC specimens from EGFR TKI treated patients reported to date. Our analysis of the clinical specimens show that AXL upregulation is the second most common mechanism of EGFR TKI acquired resistance (after EGFR T790M) in EGFR-mutant NSCLCs that has been validated using primary human data. The frequency of EGFR T790M in the EGFR TKI resistant samples in this cohort is lower than previously reported. [Engelman, J. A. et al. Science 316, 1039-1043 (2007); Arcila, M. E. et al. Clinical cancer research: an official journal of the American Association for Cancer Research 17, 1169-1180 (2011); Bean, J. et al. Proc Natl Acad Sci USA 104, 20932-20937 (2007); Sequist, L. V. et al. Sci Transl Med 3, 75ra26 (2011)] Given that the sequencing assay we used to detect EGFR T790M is standard and validated, this likely represents either distinct biology of EGFR TKI resistance in the setting of AXL or GAS6 upregulation or that there may be greater variability in the frequency of EGFR T790M in EGFR TKI resistant lung cancers than previously described. Importantly, we found upregulation of AXL in patients that developed resistance to both EGFR TKIs (erlotinib and gefitinib) that are clinically approved for use in NSCLC patients worldwide. Our findings are thus relevant to the vast majority of EGFR-mutant NSCLC patients treated with an EGFR TKI.

The data suggest that activation of AXL can occur through its overexpression as well as through upregulation of its ligand GAS6 in the setting of EGFR TKI resistance in EGFR-mutant NSCLCs. This observation is consistent with recent work showing that both upregulation of MET and its ligand HGF can promote EGFR TKI resistance in some EGFR-mutant NSCLCs. [Turke, A. B. et al. Cancer Cell 17, 77-88 (2010)] Our data showing that in some ER cell lines GAS6 is not required for erlotinib resistance are consistent with earlier work demonstrating that AXL overexpression can promote downstream signaling and induce transformation in the absence of GAS6 expression. [Burchert, A., Attar, E. C., McCloskey, P., Fridell, Y. W. & Liu, E. T. Oncogene 16, 3177-3187 (1998)] Further work will be necessary to fully elucidate the mechanisms by which GAS6 might promote EGFR TKI resistance through activation of AXL and whether somatic alterations (amplifications, rearrangements, point mutations) in AXL or GAS6 occur in human EGFR-mutant NSCLCs.

We found that in some cases AXL upregulation occurred in the context of an apparent EMT and that an EMT-associated transcriptional program involving upregulation of vimentin may, in part, drive AXL overexpression in EGFR mutant lung cancer cells with acquired EGFR TKI resistance. The data are consistent with prior studies in which vimentin upregulation was associated with AXL overexpression in breast cancer cells. [Vuoriluoto, K. et al. Oncogene 30, 1436-1448 (2011)] Although other mechanisms of AXL activation likely exist and will need to be investigated, our data are consistent with a model in which AXL may mediate acquired EGFR TKI resistance in the setting of an EMT in EGFR-mutant NSCLCs. AXL-overexpressing HCC827 ER cells exhibited increased migration and adhesion, properties associated with EMT and the metastatic behavior of tumor cells. These findings in EGFR-mutant NSCLC cells with erlotinib acquired resistance are consistent with prior work showing that overexpression of AXL is associated with increased metastasis and worse prognosis in several cancers. [Linger, R. M., Keating, A. K., Earp, H. S. & Graham, D. K. Expert Opin Ther Targets 14, 1073-1090 (2010)] Yet our studies uncover a distinct and specific role for AXL upregulation in acquired resistance to EGFR TKI treatment in EGFR-mutant NSCLC patients. Our data therefore extend current knowledge of the role of AXL as a general prognostic biomarker in human cancer and demonstrate for the first time that AXL is a biomarker of acquired resistance to EGFR-targeted therapy in NSCLC patients.

Our observation that multiple mechanisms of resistance may contribute to EGFR TKI treatment resistance in EGFR mutant NSCLC patients is consistent with other recent studies. [Bean, J. et al. Proc Natl Acad Sci USA 104, 20932-20937 (2007); Sequist, L. V. et al. Sci Transl Med 3, 75ra26 (2011)] Our data identifying AXL as a mechanism of EGFR TKI resistance enhances our understanding of tumor heterogeneity and the molecular mechanisms governing the evolution of resistance to molecularly targeted therapy in cancer patients.

Our data show that the kinase activity of AXL is required for erlotinib resistance in EGFR-mutant NSCLC tumor models. This observation provides strong rationale for the development and testing of AXL kinase inhibitors for clinical use in EGFR-mutant NSCLC patients to either prevent or overcome EGFR TKI acquired resistance. The preclinical data also suggest that activation of multiple pathways including MAPK, AKT and NF-kB may promote EGFR TKI resistance downstream of AXL upregulation. This is consistent with prior work showing that AXL can drive the growth of cancer cells through activation of each of these pathways. [Linger, R. M., Keating, A. K., Earp, H. S. & Graham, D. K. Expert Opin Ther Targets 14, 1073-1090 (2010); Keating, A. K. et al. Mol Cancer Ther 9, 1298-1307 (2010); Tai, K. Y., Shieh, Y. S., Lee, C. S., Shiah, S. G. & Wu, C. W. Oncogene 27, 4044-4055 (2008)] Further work will be necessary to determine which of these or other signaling pathways is most critical for AXL-driven EGFR TKI resistance in EGFR-mutant NSCLCs. Such studies may identify additional points for therapeutic intervention in the setting of EGFR TKI acquired resistance driven by AXL in EGFR-mutant NSCLC patients. Based on our data, we propose that inhibition of AXL signaling may enhance responses to EGFR TKI treatment in appropriately selected EGFR-mutant NSCLC patients.

TABLE 1
Erlotinib treatment response in HCC827 tumor xenografts.
Data from 10 tumors per treatment group are expressed as
average tumor volume reduction, median time to maximum
reduction, and median time to erlotinib resistance ± SEM.

| | Maximum | Time to | |
|---|---|---|---|
| | Volume | Maximum | Median |
| Erlotinib | Reduction | Volume | Time to |
| Dose | (% change | Reduction | Resistance |
| (mg/kg) | from baseline) | (weeks) | (weeks) |
| 6.25 | N/A | N/A | N/A |
| 12.5 | −56 | 4 ± 0.3 | 6 ± 0.5 |
| 25 | −69 | 5 ± 0.2 | 8 ± 0.8 |
| 50 | −84 | 4 ± 0.4 | 10 ± 1 |

TABLE 2
AXL is upregulated in human EGFR mutant NSCLC specimens from
patients with acquired EGFR TKI resistance. Clinical characteristics and expression of
the indicated biomarkers in the 35 paired EGFR-mutant NSCLC specimens obtained
from patients both prior to treatment and upon acquired resistance to treatment with
either erlotinib or gefitinib (Median PFS = 18 months, range 7-59). NA = not enough
tissue available for analysis. ACC = adenocarcinoma. NSC = non-small cell carcinoma.
SqCC = squamous cell carcinoma. VIM = vimentin.

| | | | | | AXL | | GAS6 | | | | | |
|---|---|---|---|---|---|---|---|---|---|---|---|---|
| | | | Tumor | EGFR | Base- | Resis- | Base- | Resis- | EGFR | MET | | |
| ID | Age | Sex | type | mutation | line | tance | line | tance | T790M | amp | VIM | TKI |
| 1 | 49 | M | ACC | L858R | 0 | 3 | NA | NA | − | N | NA | gefitinib |
| 2 | 50 | M | ACC | Del19 | 0 | 3 | 0 | 3 | − | N | N | gefitinib |
| 3 | 80 | M | ACC | Del19 | 0 | 3 | 0 | 3 | + | N | N | gefitinib |
| 4 | 64 | F | ACC | L858R | 1 | 3 | NA | NA | − | N | N | gefitinib |
| 5 | 47 | F | NSC | Del19 | 0 | 2 | NA | NA | − | NA | N | gefitinib |
| 6 | 48 | F | ACC | L858R | 2 | 3 | 2 | 2 | − | N | N | gefitinib |
| 7 | 59 | F | NSC | Del19 | 0 | 3 | 1 | 0 | + | N | NA | erlotinib |
| 8 | 67 | F | NSC | Del19 | 0 | 1 | 0 | 0 | + | N | NA | erlotinib |
| 9 | 74 | M | ACC | Del19 | 1 | 3 | 0 | 3 | − | N | Y | erlotinib |
| 10 | 49 | F | ACC | L858R | 2 | 3 | 2 | 3 | − | N | N | gefitinib |
| 11 | 43 | M | NSC | Del19 | 2 | 3 | 0 | 3 | − | N | N | gefitinib |
| 12 | 56 | F | NSC | Del19 | 1 | 1 | 0 | 2 | − | N | N | erlotinib |
| 13 | 54 | F | ACC | Del19 | 3 | 3 | 0 | 3 | − | N | NA | gefitinib |
| 14 | 67 | M | ACC | Del19 | 3 | 3 | 0 | 2 | − | N | NA | gefitinib |
| 15 | 59 | M | ACC | Del19 | 3 | 3 | 0 | 1 | − | NA | Y | gefitinib |
| 16 | 50 | F | ACC | Del19 | 0 | 0 | NA | NA | NA | Y | NA | gefitinib |
| 17 | 76 | F | ACC | Del19 | 0 | 0 | 3 | 3 | NA | N | NA | gefitinib |
| 18 | 64 | F | SqCC | Del19 | 0 | 0 | 0 | 0 | + | Y | NA | gefitinib |
| 19 | 66 | F | ACC | L858R | 0 | 0 | 1 | 0 | − | N | NA | gefitinib |
| 20 | 54 | F | ACC | Del19 | 0 | 0 | 0 | 0 | − | N | NA | gefitinib |
| 21 | 67 | F | ACC | L858R | 0 | 0 | NA | NA | NA | NA | NA | gefitinib |
| 22 | 73 | F | ACC | L858R | 0 | 0 | 3 | 3 | − | N | NA | gefitinib |
| 23 | 58 | M | ACC | Del19 | 0 | 0 | NA | NA | + | Y | NA | gefitinib |
| 24 | 73 | F | ACC | L858R | 0 | 0 | 2 | 2 | + | Y | NA | gefitinib |
| 25 | 62 | F | ACC | Del19 | 0 | 0 | 3 | 3 | NA | Y | NA | gefitinib |
| 26 | 77 | M | ACC | L858R | 0 | 0 | NA | NA | NA | NA | NA | gefitinib |
| 27 | 72 | F | ACC | Del19 | 0 | 0 | 2 | 3 | − | N | NA | gefitinib |
| 28 | 51 | F | ACC | L858R | 0 | 0 | 0 | 0 | − | N | NA | gefitinib |
| 29 | 67 | M | ACC | L858R | 0 | 0 | 3 | 3 | + | N | NA | gefitinib |
| 30 | 63 | M | ACC | L858R | 0 | 0 | 3 | 0 | − | N | NA | gefitinib |
| 31 | 57 | F | ACC | Del19 | 0 | 0 | 0 | 0 | NA | N | NA | gefitinib |
| 32 | 71 | F | ACC | Del19 | 0 | 0 | 0 | 0 | NA | N | NA | gefitinib |
| 33 | 54 | F | ACC | Del19 | 0 | 0 | 0 | 0 | − | N | NA | gefitinib |
| 34 | 56 | F | NSC | Del19 | 0 | 0 | 1 | 0 | − | Y | NA | gefitinib |
| 35 | 54 | F | NSC | Del19 | 0 | 0 | 0 | 0 | + | N | NA | gefitinib |

TABLE 3
Summary of resistance biomarker scoring in the paired specimens
analyzed in Table 2.

| | # | % of | Concurrent | Concurrent |
|---|---|---|---|---|
| Marker | positive | evaluable | T790M | MET |
| AXL | (7/35) | 20 | 2 | 0 |
| GAS6 | (7/28) | 25 | 1 | 0 |
| T790M | (8/28) | 29 | — | 3 |
| MET | (6/31) | 19 | 3 | — |
| Vimentin | (2/10) | 20 | 0 | 0 |

### Materials and Experimental Methods

Cell lines and reagents. The human lung cancer cell lines were purchased from the American Type Culture Collection except for H3255, PC-9 cells were generously provided. Cells were grown in RPMI 1640 supplemented with 10% FBS and 1× Antibiotic/Antimycotic (Invitrogen) and were in the logarithmic growth phase at the initiation of all experiments. Erlotinib, XL880 (GSK1363089), PHA665752 and MP-470 were purchased from Selleck Chemicals (California, USA). Drugs were dissolved in DMSO at 10 mM and stored at −20° C. The final DMSO concentration in all experiments was <0.1% in medium. All antibodies were purchased from Cell Signaling (Boston, Mass.) except the anti-AXL and anti-phospho-AXL and anti-GAS6 antibodies, which were purchased from R&D Systems (Minneapolis, Minn.).

Establishment of erlotinib-resistant subclones. Cells were exposed to increasing concentrations of erlotinib every 3 weeks from 1, 3, 5, 7, and so on until 15 μM over a 5 months period. Single-cell cloning was performed by the use of cloning cylinders and erlotinib-resistant subclones were successfully expanded with 10% fetal bovine serum culture medium containing 1 μM erlotinib. The genetic identity of each subclone with the parental cells was verified by STR (short tandem repeat) analysis according to established protocols.

Cell growth and viability assays. Cells were seeded at a density of 3000 cells/well in 96-well plates in RPMI 1640 containing 10% FBS overnight, then treated with respective agents for an additional 3 days. Viable cell numbers were determined using CellTiterGLO or MTS assay kit, as indicated in the descriptions of the drawings, according to the manufacturer's protocols (Promega, Madison, Wis., USA). Each assay consisted of 3 replicate wells and was repeated at least twice. Data were expressed as the percentage survival of control calculated from the absorbance, corrected for background.

Western blot analysis. Cells were serum starved overnight and whole cell lysates were prepared using 10% TCA lysis or RIPA buffer and clarified by centrifugation. Proteins were separated by 10% SDS-PAGE gel and transferred onto PVDF membranes (Invitrogen) for Western blot analysis. After primary antibody incubation overnight, washing and incubation with secondary antibodies, blots were developed with a chemiluminescence system (Pierce).

Gene expression profiling. Gene expression microarray profiling was performed in triplicate following RNA isolation from cells or xenograft tumors using the Qiagen RNeasy kit according to the manufacturer's instructions using either AffyMETrix U1333 2.0 plus arrays (ER1, ER2) and analyzed as described previously [Chitale, D. et al. Oncogene 28, 2773-2783 (2009); Tai, K. Y., Shieh, Y. S., Lee, C. S., Shiah, S. G. & Wu, C. W. Oncogene 27, 4044-4055 (2008)] or Illumina Human HT12-v3 arrays (ER3) and analyzed by Illumina BeadStudio Gene Expression Module v3.2. Bioinformatics analysis was performed and genes were filtered to include genes with differential expression based on setting a threshold of 3 fold change and a false discovery rate (FDR) threshold at 0.1 for the comparison of gene expression in the ER cell lines to the parental (vehicle treated) cells.

Quantitative real time RT-PCR. For the validation of genes identified by gene expression profiling, quantitative real-time RT-PCR was performed on RNA isolated from NSCLC cells. Total RNA was collected from cultured cells using PureLink Micro-to-Midi Total RNA Purification kit (Invitrogen, Carlsbad, Calif., USA). cDNA was synthesized with SuperScript III reverse transcriptase with the use of oligo(dT) primers (Invitrogen) and RT-PCR was performed by using LightCycler with Syber green probes (Roche) using the following variables: denaturation at 95° C. for 10 min, followed by 45 cycles of amplification (95° C. 10 s, 60° C. 10 s, and 72° C. 15 s), and cooling to 40° C. at a transition rate of 20° C./s. Levels of glyceraldehyde-3-phosphate dehydrogenase or actin expression were used as internal reference to normalize input cDNA. Ratios of level of each gene to as compared to reference standard were then calculated. Sequences for the primers used for AXL and GAS6 Q RT-PCR are shown in Table 4. Primers and probes used for EGFR and MET sequencing and FISH, respectively, were as previously published. [Bean, J. et al. Proc Natl Acad Sci USA 104, 20932-20937 (2007); Rosell, R., Wei, J. & Taron, M. Clin Lung Cancer 10, 8-9 (2009)]

siRNA knockdown. Knockdown of AXL, MET and vimentin was performed using specific single or pooled siRNAs, as indicated, targeting the indicated genes purchased from Dharmacon RNAi Technologies (Thermo Scientific, Rockford, Ill.). SiGENOME non-targeting siRNAs served as negative controls. Introduction of siRNA was performed with DharmaFect1 reagent according to the manufacturer's instructions. Efficiency of knockdown at different times or dose points was assessed by Q RT PCR or western blotting on cell lysates.

AXL cDNA and overexpression studies. The AXL gene with an HA tag at the C-terminus was amplified by overlapping PCR using cDNA generated from ER3 cells and cloned into pcDNA3.1 (+) vector. Amino acid changes (K567R or L620M) were introduced using the Strategene QuickChange Mutagenesis Kit, according to the manufacturer's protocol. The accuracy of the constructs was confirmed by DNA sequencing. Cells were transfected with the AXL-expressing vectors or empty vector as a control by using FuGENE HD reagent according to the manufacturer's protocol (Roche Applied Sciences, Indianapolis, Ind., USA). Stable HCC827 subclones resistant to the selection antibiotic, 500 μg/ml G418 were generated 24 hours post-transfection. Empty-vector expressing HCC827 cells were generated as a control. AXL protein expression was detected by using anti-HA probe (Santa Cruz Biotechnology).

Migration and adhesion studies. 180,000 cells were plated onto 3.5 cm cell culture dishes. At 30 minutes, the dishes were washed and cells were fixed and stained for counting. Average cell numbers of 5 random 100× fields under light microscope are presented with ±SEM. Migration: 10,000 cells in 200 μl medium without FBS were placed in the upper chamber of transwells (6.5 mm diameter, 8 μm pore size polycarbonate membrane, Corning), and the lower chamber filled with 1 ml of medium with 10% FBS supplemented with or without the indicated drug treatments. After incubation for 16 hours, non-migrating cells were removed with cotton swabs, and the cells that migrated into the lower surface of the filters were stained with crystal violet. Cells were counted under a microscope, and triplicate results are expressed as means±SEM.

shRNA knockdown. For the shRNA experiments each retroviral and lentiviral shRNA was packaged in 293FT cells according to the manufacturer's instructions. Indicated cell lines were spin infected with 1 ug/ml polybrene and 48 h after infection were selected with 2 μg/ml of puromycin for 48 h. Target gene expression was measured by either Q RT PCR or western blot 48-72 h later.

Tumorigenesis assays. Human lung cancer cells (as indicated) were injected subcutaneously into the flanks of CB17 SCID mice (Taconic). Tumor-bearing mice (tumor size 200-500 mm³) were randomized to treatment with vehicle (DMSO/CMC) or erlotinib HCL 12.5 mg/kg/day intraperitoneally. Tumor measurements were made on days indicated and expressed as described in the descriptions of the drawings. All animal experiments were performed in compliance with the guidelines of the Research Animal Resource Center of the Memorial Sloan-Kettering Cancer Center.

Combination index (CI) analysis. The combination effects were evaluated with the MTT assay at a 1:1 ratio (erlotinib (μM):XL880 (μM) and erlotinib (μM):PHA665752 (μM)) in HCC827ER cells. The fraction affected (Fa) (i.e., Fa of 0.25 is equivalent to 75% viable cells) and CI values processed using CalcuSyn software (Biosoft, Cambridge, UK). CI values of less than 1, 1, and greater than 1 were taken as synergism, additive effect, and antagonism, respectively.

Immunoprecipitation. For immunoprecipitation, 1 mg protein from total lysates was mixed with 1 μg of anti-AXL (Santa Cruz, Calif.), and incubated on ice for 2 h. Protein G Sepharose Fast Flow (GE Healthcare, Little Chalfont, Buckinghamshire, UK) was added to the mixture and incubated overnight at 4° C. Immune complexes were pelleted by centrifugation at 3000 rpm for 5 min, washed twice with lysis buffer, and resuspended in 70 μl of SDS-PAGE sample buffer. Subsequently, immune complexes were probed with anti-AXL and phosphotyrosine antibodies (Santa Cruz, Calif.).

Sequencing of AXL, EGFR, K-Ras and B-Raf. Sequencing of full length AXL and EGFR was performed by standard methods using the primer sequences listed in Table 5. Sequencing of exon 2 of K-Ras and exons 11 and 15 of BRAF was performed on genomic DNA using validated primers and protocols. [Bean, J. et al. Proc Natl Acad Sci USA 104, 20932-20937 (2007)]

IHC on NSCLC clinical specimens. All specimens were acquired from patients under the auspices of IRB-approved clinical protocols at each hospital in which informed consent was obtained and were formalin-fixed, paraffin-embedded tumour tissues (FFPE), that were examined to ensure >75% tumor infiltration, and stained with haematoxilin/eosin and assessed by a thoracic pathologist. IHC was performed in the Core Research IHC Laboratory at each institution on FFPE sections, as previously described [Brevet, M., Arcila, M. & Ladanyi, M. The Journal of molecular diagnostics: JMD 12, 169-176 (2010)], using the indicated antibodies: Human AXL antigen—affinity-purified polyclonal antibody, R&D systems, catalog #AF154AXL; Human GAS6—affinity-purified polyclonal antibody, R&D systems, catalog #AB885, Human vimentin—affinity-purified polyclonal antibody, Cell Signaling Technologies, catalog #3932. Expression was examined by standard immunohistochemistry using 4 μm thick sections of matched, paired specimens. Deparaffinized tissue sections were stained following the manufacturer's protocol at a dilution of 1:500 on a Ventana Dixcovery XT automated stainer. EGFR T790M mutation and MET amplification were analyzed using previously described sequencing and FISH protocols, [Bean, J. et al. Proc Natl Acad Sci USA 104, 20932-20937 (2007)] respectively, and vimentin expression by IHC using established methods and clinical scoring criteria. [Azumi, N. & Battifora, H. American journal of clinical pathology 88, 286-296 (1987)]

Sequencing of exon 2 of K-Ras and exons 11 and 15 of B-Raf was performed on isolated genomic DNA using primers and protocols previously published. [Bean, J. et al. Proc Natl Acad Sci USA 104, 20932-20937 (2007)]

TABLE 4
Human AXL and GAS6 Q RT PCR primer sequences.
Gene

| Target | Sense | Anti-sense |
|---|---|---|
| AXL | 5′-AGACATCGCCAGTGGCATG-3′ | 5′-AGGCGATTTCCTGCTTCAGG-3′ |
| | (SEQ ID NO: 1) | (SEQ ID NO: 2) |
| GAS6 | 5′-CATCAACAAGTATGGGTCTCCGT-3′ | 5′-GTTCTCCTGGCTGCATTCGTTGA-3′ |
| | (SEQ ID NO: 3) | (SEQ ID NO: 4) |

TABLE 5
Human AXL and EGFR DNA sequencing primer sequences.

| | |
|---|---|
| AXL | 5′-TGAAGAAAGTCCCTTCGTGG-3′, |
| | (SEQ ID NO: 5) |
| | 5′-GATCTGTCCATCCCGAAGCC-3′ |
| | (SEQ ID NO: 6) |
| | 5′-TGTCAGACGATGGGATGGGC-3′, |
| | (SEQ ID NO: 7) |
| | 5′-GCGTCTCCACAGGAAGCCAG-3′ |
| | (SEQ ID NO: 8) |
| | 5′-TGGTAGTCAGGTACCGCGTG-3′, |
| | (SEQ ID NO: 9) |
| | 5′-TCCAGCTCTGACCTCGTGCAG-3′ |
| | (SEQ ID NO: 10) |
| | 5′-ATATCCGGGCGTGGAGAACAGC-3′, |
| | (SEQ ID NO: 11) |
| | 5′-GAATCCTTAGGGTCTGGCTG-3′ |
| | (SEQ ID NO: 12) |
| EGFR | 5′-CTGCGTGAGCTTGTTACTCGTGCCTTGG-3′, |
| | (SEQ ID NO: 13) |
| | 5′-AGCAGTCACTGGGGGACTTG-3′ |
| | (SEQ ID NO: 14) |
| | 5′-GGTGCAGGAGAGGAGAACTGC-3′, |
| | (SEQ ID NO: 15) |
| | 5′-GGTTTTCTGACCGGAGGTCC-3′ |
| | (SEQ ID NO: 16) |
| | 5′-AGGACCAAGCAACATGGTCAG-3′, |
| | (SEQ ID NO: 17) |
| | 5′-TGCATCCGTAGGTGCAGTTTG-3′ |
| | (SEQ ID NO: 18) |
| | 5′-GATGGTGGGGGCCCTCCTCTT-3′, |
| | (SEQ ID NO: 19) |
| | 5′-TCCGGGAACACAAAGACAATA-3′ |
| | (SEQ ID NO: 20) |
| | 5′-CTTTCTCTTCCGCACCCAGCAGTT-3′, |
| | (SEQ ID NO: 21) |
| | 5′-ATCCATCAGGGCACGGTAGAAGTT-3′ |
| | (SEQ ID NO: 22) |
| | 5′-AGTGCTGGATGATAGACGCAG-3′, |
| | (SEQ ID NO: 23) |
| | 5′-GTCAACAGCACATTCGACAGC-3′ |
| | (SEQ ID NO: 24) |
| | 5′-AAATTCACTGCTTTGTGGCGC-3′ |
| | (SEQ ID NO: 25) |

TABLE 6
76 gene EMT signature, including AXL, with accompanying gene
symbols, accession numbers, and/or Affymetrix probe numbers for
identifying the EMT Markers making up an EMT signature, including
AXL, by which epithelial cells and mesenchymal cells may be
differentiated. EMT Markers and AXL identified by Affymetrix Probe
number, Accession number, Gene Symbol, and/or Gene Name.

| Affymetrix | | Gene | |
|---|---|---|---|
| Probe | Accession | Symbol | Gene Name |
| 228441_s_at | AC092611 | | |
| 229842_at | AC099676 | | |
| 235144_at | AK056882 | 9q21.32 | “CDNA FLI32320 fis, clone |
| | | | PROST2003537” |
| 235988_at | AB065679 | | |
| 236279_at | AC010503 | | |
| 236489_at | AB065679 | | |
| 238742_x_at | | | |
| 239148_at | AC009097 | | |
| 242354_at | | | |
| 238439_at | NM_144590 | ANKRD22 | Ankyrin repeat domain 22 |
| 225524_at | NM_058172 | ANTXR2 | Anthrax toxin receptor 2 |
| 65517_at | NM_005498 | AP1M2 | “Adaptor-related protein complex 1, |
| | | | mu 2 subunit” |
| 218261_at | NM_005498 | AP1M2 | “Adaptor-related protein complex 1, |
| | | | mu 2 subunit” |
| 202686_s_at | NM_021913 | AXL | AXL receptor tyrosine kinase |
| 218792_s_at | NM_017688 | BSPRY | B-box and SPRY domain containing |
| 222746_s_at | NM_017688 | BSPRY | B-box and SPRY domain containing |
| 228865_at | NM_023938 | C1orf116 | Chromosome 1 open reading frame |
| | | | 116 |
| 236058_at | NM_152365 | C1orf172 | Chromosome 1 open reading frame |
| | | | 172 |
| 226891_at | NM_152531 | C3orf21 | Chromosome 3 open reading frame 21 |
| 224414_s_at | NM_032587 | CARD6 | “Caspase recruitment domain family, |
| | | | member 6” |
| 201131_s_at | NM_004360 | CDH1 | |
| 203256_at | NM_001793 | CDH3 | “Cadherin 3, type 1, P-cadherin |
| | | | (placental)” |
| 205709_s_at | NM_001263 | CDS1 | CDP-diacylglycerol synthase |
| | | | (phosphatidate cytidylyltransferase)1 |
| 226185_at | AK026697 | CDS1 | “CDNA; FLJ23044 fis, clone |
| | | | LNG02454” |
| 226187_at | AK026697 | CDS1 | “CDNA; FLJ23044 fis, clone |
| | | | LNG02454” |
| 201428_at | NM_001305 | CLDN4 | Claudin 4 |
| 202790_at | NM_001307 | CLDN7 | Claudin 7 |
| 232609_at | NM_174881 | CRB3 | Crumbs homolog 3 (Drosophila) |
| 200606_at | NM_004415 | DSP | Desmoplakin |
| 219411_at | NM_024712 | ELM03 | Engulfment and cell motility 3 |
| 227803_at | NM_021572 | ENPP5 | Ectonucleotide |
| | | | pyrophosphatase/phosphodiesterase 5 |
| | | | (putative function) |
| 229292_at | BC032822 | EPB41L5 | Erythrocyte membrane protein band |
| | | | 4.1 like 5 |
| 205977_s_at | NM_005232 | EPHA1 | EPH receptor A1 |
| 220318_at | NM_017957 | EPN3 | Epsin 3 |
| 223895_s_at | NM_017957 | EPN3 | Epsin 3 |
| 232164_s_at | NM_031308 | EPPK1 | Epiplakin 1 |
| 232165_at | AL137725 | EPPK1 | Epiplakin 1 |
| 202454_s_at | NM_001982 | ERBB3 | V-erb-b2 erythroblastic leukemia viral |
| | | | oncogene homolog 3 (avian) |
| 204503_at | NM_001988 | EVPL | Envoplakin |
| 224097_s_at | NM_144504 | F11R | |
| 211719_x_at | NM_212482 | FN1 | Fibronectin 1 |
| 214702_at | NM_054034 | FN1 | Fibronectin 1 |
| 202489_s_at | NM_021910 | FXYD3 | FXYD domain containing ion |
| | | | transport regulator 3 |
| 203397_s_at | NM_004482 | GALNT3 | UDP-N-acetyl-alpha-D- |
| | | | galactosamine:polypeptide N- |
| | | | acetylgalactosaminyltransferase 3 |
| | | | (GalNAc-T3) |
| 229555_at | NM_014568 | GALNT5 | UDP-N-acetyl-alpha-D- |
| | | | galactosamine:polypeptide N- |
| | | | acetylgalactosaminyltransferase 5 |
| | | | (GalNAc-T5) |
| 238689_at | NM_153840 | GPR110 | G protein-coupled receptor 110 |
| 212070_at | NM_201525 | GPR56 | G protein-coupled receptor 56 |
| 222830_at | NM_198182 | GRHL1 | Grainyhead-like 1 (Drosophila) |
| 219388_at | NM_024915 | GRHL2 | Grainyhead-like 2 (Drosophila) |
| 204112_s_at | NM_006895 | HNMT | Histamine N-methyltransferase |
| 211732_x_at | NM_00102407 | HNMT | Histamine N-methyltransferase |
| 223681_s_at | AB044807 | INADL | |
| 226535_at | AK026736 | ITGB6 | “Integrin, beta 6” |
| 239853_at | NM_177417 | KLC3 | Kinesin light chain 3 |
| 201650_at | NM_002276 | KRT19 | Keratin 19 |
| 235148_at | NM_173853 | KRTCAP3 | Keratinocyte associated protein 3 |
| 225793_at | AK128733 | LIX1L | Lix1 homolog (mouse)-like |
| 219476_at | AK058009 | LRRC54 | “CDNA FLJ25280 fis, clone |
| | | | STM06543” |
| 224650_at | NM_052886 | MAL2 | “Mal, T-cell differentiation protein 2” |
| 210058_at | NM_002754 | MAPK13 | Mitogen-activated protein kinase 13 |
| 201069_at | NM_004530 | MMP2 | “Matrix metallopeptidase 2 (gelatinase |
| | | | A, 72 kDa gelatinase, 72 kDa type IV |
| | | | collagenase)” |
| 238778_at | AL832380 | MPP7 | “Membrane protein, palmitoylated 7 |
| | | | (MAGUK p55 subfamily member 7)” |
| 203780_at | NM_005797 | MPZL2 | Myelin protein zero-like 2 |
| 234970_at | | MTAC2D1 | |
| 207847_s_at | NM_002456 | MUC1 | “Mucin 1, cell surface associated” |
| 212298_at | NM_003873 | NRP1 | Neuropilin 1 |
| 208510_s_at | NM_015869 | PPARG | Peroxisome proliferator-activated |
| | | | receptor gamma |
| 37117_at | NM_00101752 | PRR5 | Rho GTPase activating protein 8 |
| 205980_s_at | NM_00101752 | PRR5 | Rho GTPase activating protein 8 |
| 205847_at | NM_022119 | PRSS22 | “Protease, serine, 22” |
| 202525_at | NM_002773 | PRSS8 | “Protease, serine, 8” |
| 218186_at | NM_020387 | RAB25 | “RAB25, member RAS oncogene |
| | | | family” |
| 219121_s_at | NM_017697 | RBM35A | RNA binding motif protein 35A |
| 225846_at | NM_00103491 | RBM35A | RNA binding motif protein 35A |
| 209488_s_at | NM_00100871 | RBPMS | RNA binding protein with multiple |
| | | | splicing |
| 218677_at | NM_020672 | S100A14 | S100 calcium binding protein A14 |
| 203453_at | NM_001038 | SCNN1A | “Sodium channel, nonvoltage-gated 1 |
| | | | alpha” |
| 224762_at | NM_178865 | SERINC2 | Serine incorporator 2 |
| 204019_s_at | NM_015677 | SH3YL1 | “SH3 domain containing, Ysc84-like 1 |
| | | | (S. cerevisiae)” |
| 225548_at | NM_020859 | SHROOM3 | Shroom family member 3 |
| 210715_s_at | NM_021102 | SPINT2 | “Serine peptidase inhibitor, Kunitz |
| | | | type, 2” |
| 219919_s_at | NM_018276 | SSH3 | |
| 202005_at | NM_021978 | ST14 | Suppression of tumorigenicity 14 |
| | | | (colon carcinoma) |
| 216905_s_at | NM_021978 | ST14 | Suppression of tumorigenicity 14 |
| | | | (colon carcinoma) |
| 221610_s_at | NM_00101384 | STAP2 | Signal transducing adaptor family |
| | | | member 2 |
| 201839_s_at | NM_002354 | TACSTD1 | Tumor-associated calcium signal |
| | | | transducer 1 |
| 202286_s_at | NM_002353 | TACSTD2 | Tumor-associated calcium signal |
| | | | transducer 2 |
| 201506_at | NM_000358 | TGFBI | “Transforming growth factor, beta- |
| | | | induced, 68 kDa” |
| 35148_at | NM_014428 | TJP3 | Tight junction protein 3 (zona |
| | | | occludens 3) |
| 226403_at | NM_144686 | TMC4 | Transmembrane channel-like 4 |
| 225822_at | NM_144626 | TMEM125 | Transmembrane protein 125 |
| 213285_at | NM_00101797 | TMEM30B | Transmembrane protein 30B |
| 226226_at | NM_138788 | TMEM45B | Transmembrane protein 45B |
| 218856_at | NM_014452 | TNFRSF21 | “Tumor necrosis factor receptor |
| | | | superfamily, member 21” |
| 201426_s_at | NM_003380 | VIM | Vimentin |
| 210875_s_at | NM_030751 | ZEB1 | Zinc finger E-box binding homeobox 1 |
| 212764_at | BX647794 | ZEB1 | Zinc finger E-box binding homeobox 1 |

TABLE 7
35 gene EMT signature, including AXL, with accompanying gene
symbols, accession numbers, and/or Affymetrix probe numbers for
identifying the EMT Markers making up an EMT signature, including
AXL, by which epithelial cells and mesenchymal cells may be
differentiated. EMT Markers and AXL identified by Affymetrix Probe
number, Accession number, Gene Symbol, and/or Gene Name.

| Affymetrix | | Gene | |
|---|---|---|---|
| Probe | Accession | Symbol | Gene Name |
| 238439_at | NM_144590 | ANKRD22 | Ankyrin repeat domain 22 |
| 225524_at | NM_058172 | ANTXR2 | Anthrax toxin receptor 2 |
| 202686_s_at | NM_021913 | AXL | AXL receptor tyrosine kinase |
| 228865_at | NM_023938 | C1orf116 | Chromosome 1 open reading frame |
| | | | 116 |
| 203256_at | NM_001793 | CDH3 | “Cadherin 3, type 1, P-cadherin |
| | | | (placental)” |
| 205709_s_at | NM_001263 | CDS1 | CDP-diacylglycerol synthase |
| | | | (phosphatidate cytidylyltransferase)1 |
| 202790_at | NM_001307 | CLDN7 | Claudin 7 |
| 205977_s_at | NM_005232 | EPHA1 | EPH receptor A1 |
| 232164_s_at | NM_031308 | EPPK1 | Epiplakin 1 |
| 232165_at | AL137725 | EPPK1 | Epiplakin 1 |
| 202454_s_at | NM_001982 | ERBB3 | V-erb-b2 erythroblastic leukemia |
| | | | viral oncogene homolog 3 (avian) |
| 224097_s_at | NM_144504 | F11R | |
| 238689_at | NM_153840 | GPR110 | G protein-coupled receptor 110 |
| 212070_at | NM_201525 | GPR56 | G protein-coupled receptor 56 |
| 219388_at | NM_024915 | GRHL2 | Grainyhead-like 2 (Drosophila) |
| 204112_s_at | NM_006895 | HNMT | Histamine N-methyltransferase |
| 211732_x_at | NM_00102407 | HNMT | Histamine N-methyltransferase |
| 201650_at | NM_002276 | KRT19 | Keratin 19 |
| 235148_at | NM_173853 | KRTCAP3 | Keratinocyte associated protein 3 |
| 224650_at | NM_052886 | MAL2 | “Mal, T-cell differentiation protein |
| | | | 2” |
| 210058_at | NM_002754 | MAPK13 | Mitogen-activated protein kinase 13 |
| 207847_s_at | NM_002456 | MUC1 | “Mucin 1, cell surface associated” |
| 208510_s_at | NM_015869 | PPARG | Peroxisome proliferator-activated |
| | | | receptor gamma |
| 202525_at | NM_002773 | PRSS8 | “Protease, serine, 8” |
| 218186_at | NM_020387 | RAB25 | “RAB25, member RAS oncogene |
| | | | family” |
| 218677_at | NM_020672 | S100A14 | S100 calcium binding protein A14 |
| 203453_at | NM_001038 | SCNN1A | “Sodium channel, nonvoltage-gated 1 |
| | | | alpha” |
| 204019_s_at | NM_015677 | SH3YL1 | “SH3 domain containing, Ysc84-like |
| | | | 1 (S. cerevisiae)” |
| 210715_s_at | NM_021102 | SPINT2 | “Serine peptidase inhibitor, Kunitz |
| | | | type, 2” |
| 202005_at | NM_021978 | ST14 | Suppression of tumorigenicity 14 |
| | | | (colon carcinoma) |
| 216905_s_at | NM_021978 | ST14 | Suppression of tumorigenicity 14 |
| | | | (colon carcinoma) |
| 201839_s_at | NM_002354 | TACSTD1 | Tumor-associated calcium signal |
| | | | transducer 1 |
| 201506_at | NM_000358 | TGFBI | “Transforming growth factor, beta- |
| | | | induced, 68 kDa” |
| 226403_at | NM_144686 | TMC4 | Transmembrane channel-like 4 |
| 225822_at | NM_144626 | TMEM125 | Transmembrane protein 125 |
| 213285_at | NM_00101797 | TMEM30B | Transmembrane protein 30B |
| 218856_at | NM_014452 | TNFRSF21 | “Tumor necrosis factor receptor |
| | | | superfamily, member 21” |
| 201426_s_at | NM_003380 | VIM | Vimentin |

TABLE 8
35 gene EMT signature, including AXL, with accompanying gene
symbols, accession numbers, and/or Affymetrix probe numbers for
identifying the EMT Markers making up an EMT signature, including
AXL, by which epithelial cells and mesenchymal cells may be
differentiated. EMT Markers and AXL identified by Affymetrix Probe
number, Accession number, Gene Symbol, and/or Gene Name.

| Affymetrix | | Gene | |
|---|---|---|---|
| Probe | Accession | Symbol | Gene Name |
| 238439_at | NM_144590 | ANKRD22 | Ankyrin repeat domain 22 |
| 225524_at | NM_058172 | ANTXR2 | Anthrax toxin receptor 2 |
| 202686_s_at | NM_021913 | AXL | AXL receptor tyrosine kinase |
| 228865_at | NM_023938 | C1orf116 | Chromosome 1 open reading frame |
| | | | 116 |
| 203256_at | NM_001793 | CDH3 | “Cadherin 3, type 1, P-cadherin |
| | | | (placental)” |
| 226185_at | AK026697 | CDS1 | “CDNA; FLJ23044 fis, clone |
| | | | LNG02454” |
| 226187_at | AK026697 | CDS1 | “CDNA; FLJ23044 fis, clone |
| | | | LNG02454” |
| 202790_at | NM_001307 | CLDN7 | Claudin 7 |
| 205977_s_at | NM_005232 | EPHA1 | EPH receptor A1 |
| 232164_s_at | NM_031308 | EPPK1 | Epiplakin 1 |
| 232165_at | AL137725 | EPPK1 | Epiplakin 1 |
| 202454_s_at | NM_001982 | ERBB3 | V-erb-b2 erythroblastic leukemia |
| | | | viral oncogene homolog 3 (avian) |
| 224097_s_at | NM_144504 | F11R | |
| 238689_at | NM_153840 | GPR110 | G protein-coupled receptor 110 |
| 212070_at | NM_201525 | GPR56 | G protein-coupled receptor 56 |
| 219388_at | NM_024915 | GRHL2 | Grainyhead-like 2 (Drosophila) |
| 204112_s_at | NM_006895 | HNMT | Histamine N-methyltransferase |
| 211732_x_at | NM_00102407 | HNMT | Histamine N-methyltransferase |
| 201650_at | NM_002276 | KRT19 | Keratin 19 |
| 235148_at | NM_173853 | KRTCAP3 | Keratinocyte associated protein 3 |
| 224650_at | NM_052886 | MAL2 | “Mal, T-cell differentiation protein |
| | | | 2” |
| 210058_at | NM_002754 | MAPK13 | Mitogen-activated protein kinase 13 |
| 207847_s_at | NM_002456 | MUC1 | “Mucin 1, cell surface associated” |
| 208510_s_at | NM_015869 | PPARG | Peroxisome proliferator-activated |
| | | | receptor gamma |
| 202525_at | NM_002773 | PRSS8 | “Protease, serine, 8” |
| 218186_at | NM_020387 | RAB25 | “RAB25, member RAS oncogene |
| | | | family” |
| 218677_at | NM_020672 | S100A14 | S100 calcium binding protein A14 |
| 203453_at | NM_001038 | SCNN1A | “Sodium channel, nonvoltage-gated 1 |
| | | | alpha” |
| 204019_s_at | NM_015677 | SH3YL1 | “SH3 domain containing, Ysc84-like |
| | | | 1 (S. cerevisiae)” |
| 210715_s_at | NM_021102 | SPINT2 | “Serine peptidase inhibitor, Kunitz |
| | | | type, 2” |
| 202005_at | NM_021978 | ST14 | Suppression of tumorigenicity 14 |
| | | | (colon carcinoma) |
| 216905_s_at | NM_021978 | ST14 | Suppression of tumorigenicity 14 |
| | | | (colon carcinoma) |
| 201839_s_at | NM_002354 | TACSTD1 | Tumor-associated calcium signal |
| | | | transducer 1 |
| 201506_at | NM_000358 | TGFBI | “Transforming growth factor, beta- |
| | | | induced, 68 kDa” |
| 226403_at | NM_144686 | TMC4 | Transmembrane channel-like 4 |
| 225822_at | NM_144626 | TMEM125 | Transmembrane protein 125 |
| 213285_at | NM_00101797 | TMEM30B | Transmembrane protein 30B |
| 218856_at | NM_014452 | TNFRSF21 | “Tumor necrosis factor receptor |
| | | | superfamily, member 21” |
| 201426_s_at | NM_003380 | VIM | Vimentin |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of detecting an increased level of AXL or GAS6 in a cancer patient, the method comprising:
(i) obtaining a sample from a cancer patient; and
(ii) detecting an increased level of AXL or GAS6 in said sample relative to a control.

2. The method of claim 1, wherein said cancer patient has a cancer comprising cancer cells with an EGFR-activating mutation.

3. The method of claim 1, wherein the EGFR-activating mutation is an exon-19 deletion or an exon-21 point mutant.

4. The method of claim 1, wherein the method does not comprise detecting a level of an EMT marker except AXL.

5. The method of claim 1, wherein said detecting comprises:
(a) contacting the sample with a detectable AXL-binding agent or detectable GAS6-binding agent;
(b) allowing said detectable AXL-binding agent and AXL to form an AXL complex or said GAS6-binding agent and GAS6 to form a GAS6 complex; and
(c) detecting said AXL complex or GAS6 complex.

6. The method of claim 1, wherein said detecting comprises detecting a level of an AXL or GAS6 nucleic acid or fragment thereof.

7. The method of claim 1, wherein said detecting comprises use of nucleic acid amplification, a gene array, a microarray, a macroarray, a DNA array, or a DNA chip.

8. The method of claim 1, wherein said detecting comprises detecting a level of an AXL or GAS6 protein or fragment thereof.

9. The method of claim 1, wherein said detecting comprises use of an antibody, flow cytometry, ELISA, mass spectroscopy, immunofluorescence, or fluorescence microscopy.

10. The method of claim 9, wherein said antibody is conjugated to a detectable moiety.

11. The method of claim 1 comprising detecting an increased level of AXL.

12. The method of claim 1, wherein the level of AXL is a level of AXL mRNA, AXL protein, or AXL kinase activity.

13. The method of claim 1, wherein said cancer patient is an EGFR TKI resistant cancer patient, wherein said EGFR TKI resistant cancer patient has a cancer that is not a mesenchymal cancer.

14. The method of claim 13, wherein said EGFR TKI resistant cancer patient is resistant to gefitinib, erlotinib, cetuximab, lapatinib, panitumumab, vandetanib, afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626, zalutumumab, nimotuzumab, matuzumab, AP26113, or CO-1686.

15. The method of claim 13, wherein the EGFR TKI resistant cancer patient is resistant to erlotinib or gefitinib.

16. The method of claim 13, wherein said EGFR TKI resistant cancer patient has a cancer selected from the group consisting of lung cancer, pancreatic cancer, breast cancer, colon cancer, esophageal cancer, thyroid cancer, liver cancer, glioblastoma, and astrocytoma-glioblastoma.

17. The method of claim 16, wherein said lung cancer is non-small cell lung cancer (NSCLC).

18. The method of claim 16, wherein said cancer is metastatic cancer.

19. The method of claim 16, wherein said cancer is non-small cell lung cancer comprising EGFR having an exon-19 deletion or an exon-21 point mutant; and further wherein the cancer is erlotinib resistant or gefitinib resistant relative to a control.

20. A method of identifying an EGFR inhibitor resistant cancer patient comprising:
(i) obtaining a sample from a cancer patient;
(ii) detecting an increased level of AXL or GAS6 in said sample relative to a control.

21. The method of claim 20, wherein said EGFR inhibitor resistant cancer patient has a cancer comprising cancer cells with an EGFR-activating mutation.

22. The method of claim 21, wherein the EGFR-activating mutation is an exon-19 deletion or an exon-21 point mutant.

23. The method of claim 20, wherein the method does not comprise detecting a level of an EMT marker except AXL.

24. The method of claim 20, wherein said EGFR inhibitor resistant cancer patient has a cancer that is not a mesenchymal cancer.

25. The method of claim 20, wherein said detecting comprises detecting a level of an AXL or GAS6 nucleic acid or fragment thereof.

26. The method of claim 25, wherein said detecting comprises use of nucleic acid amplification, a gene array, a microarray, a macroarray, a DNA array, or a DNA chip.

27. The method of claim 20, wherein said detecting comprises detecting a level of an AXL or GAS6 protein or fragment thereof.

28. The method of claim 27, wherein said detecting comprises use of an antibody, flow cytometry, ELISA, mass spectroscopy, immunofluorescence, or fluorescence microscopy.

29. The method of claim 28, wherein said antibody is conjugated to a detectable moiety.

30. The method of claim 20, wherein said EGFR inhibitor resistant cancer patient is resistant to gefitinib, erlotinib, cetuximab, lapatinib, panitumumab, vandetanib, afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626, zalutumumab, nimotuzumab, matuzumab, AP26113, or CO-1686.

31. The method of claim 20, wherein the EGFR inhibitor resistant cancer patient is resistant to erlotinib or gefitinib.

32. The method of claim 20, wherein said EGFR inhibitor resistant cancer patient has a cancer selected from the group consisting of lung cancer, pancreatic cancer, breast cancer, colon cancer, esophageal cancer, thyroid cancer, liver cancer, glioblastoma, and astrocytoma-glioblastoma.

33. The method of claim 32, wherein said lung cancer is non-small cell lung cancer (NSCLC).

34. The method of claim 32, wherein said cancer is metastatic cancer.

35. The method of claim 32, wherein said cancer is non-small cell lung cancer comprising EGFR having an exon-19 deletion or an exon-21 point mutant; and further wherein the cancer is erlotinib resistant or gefitinib resistant relative to a control.

36. The method of claim 20 comprising detecting an increased level of AXL.

37. The method of claim 20, wherein the level of AXL is a level of AXL mRNA, AXL protein, or AXL kinase activity.
